(19) United States
(12) Patent Application Publication
(10) Pub. No.: US 2016/0369292 A1
(43) Pub. Date: Dec. 22, 2016

(54) RECOMBINANT PLANTS AND MICROORGANISMS HAVING A REVERSE GLYOXYLATE SHUNT

(71) Applicant: THE REGENTS OF THE UNIVERSITY OF CALIFORNIA, Oakland, CA (US)

(72) Inventors: James C. Liao, Los Angeles, CA (US); Luisa Gronenberg, Los Angeles, CA (US); Samuel Mainguet, Los Angeles, CA (US); Sio Si Wong, Los Angeles, CA (US); Avinash Srivastava, Los Angeles, CA (US)

(21) Appl. No.: 14/901,278

(22) PCT Filed: Jun. 29, 2014
(86) PCT No.: PCT/US2014/044772

§ 371 Date: Dec. 28, 2015

Related U.S. Application Data
(60) Provisional application No. 61/841,310, filed on Jun. 29, 2013.

(51) Int. Cl. C12N 15/82; C12P 19/32; C12P 7/48; C12N 9/00; C12N 9/88

(52) U.S. Cl. CPC C12N 15/8261; C12N 9/93; C12N 9/88; C12P 7/48; C12P 19/32; C12Y 602/01009; C12Y 401/03001; C12Y 401/03024

(57) ABSTRACT

Provided are microorganisms and plants that express or overexpress enzymes that catalyze the conversion of a four carbon metabolite (malate) to acetyl-CoA. Also provided are methods of generating such organisms and plants and methods of synthesizing biomass, biofuel, oil, chemicals and biochemicals using such organisms and plants.

### CROSS REFERENCE TO RELATED APPLICATIONS

This application claims priority to U.S. Provisional Application Ser. No. 61/841,310, filed Jun. 29, 2013, the disclosure of which is incorporated herein by reference in its entirety.

### STATEMENT REGARDING FEDERALLY SPONSORED RESEARCH

This invention was made with Government support under Grant Nos. DE-AR0000085 and DE-AR0000201, awarded by the U.S. Department of Energy. The Government has certain rights in the invention.

### TECHNICAL FIELD

Metabolically-modified microorganisms and plants and methods of producing such organisms and plants are provided. Also provided are methods of producing chemicals by contacting a suitable substrate with a metabolically-modified microorganism or plant and enzymatic preparations of the disclosure.

### BACKGROUND

Acetyl-CoA is a central metabolic key to both cell growth as well as biosynthesis of multiple cell constituents and products, including fatty acids, amino acids, isoprenoids, and alcohols. Typically, the Embden-Meyerhof-Parnas (EMP) pathway, the Entner-Doudoroff (ED) pathway, and their variations are used to produce acetyl-CoA from sugars through oxidative decarboxylation of pyruvate.

Most central metabolic pathways such as glycolysis, fatty acid synthesis, and the TCA cycle have complementary pathways that run in the reverse direction to allow flexible storage and utilization of resources. However, the glyoxylate shunt, which allows for the synthesis of four-carbon TCA cycle intermediates from acetyl-CoA, has not been found to be reversible to date. As a result, glucose can only be converted to acetyl-CoA via the decarboxylation of the three-carbon molecule pyruvate in heterotrophs.

Genetic modification of plants has, in combination with conventional breeding programs, led to significant increases in agricultural yield over the last decades. Genetically modified plants may be selected for one or more agronomic traits, for example by expression of enzyme coding sequences (e.g., enzymes that provide herbicide resistance). Genetic manipulation of genes involved in plant growth or yield may enable increased production of valuable commercial crops, resulting in agricultural benefits and development of alternate energy sources such as biofuels.

Plant biomass content has recently become an intense area of research due to the broad ranging commercial applications and plant biomass is directly related to photosynthetic efficiency. Significant improvement in the photosynthetic rate can play a vital role in not only increasing the plant biomass but it can lead to a healthy life style for everyone as a healthy plant can cater our nutritional needs in a better manner. Development of plants with modified or improved photosynthetic rates would have a significant benefit for the production of biofuels and animal feeds as well and could potentially have a broad range of other beneficial applications. However genetic modification of plants to achieve these goals by improving photosynthetic machinery has not been realized.

A major stumbling block to increase the photosynthesis in plants is Rubisco, an enzyme that can use O₂ and CO₂ both as substrates. Due to high oxygenase activity, plants normally underperform and never reach optimum level of productivity. Over the years, plant science researchers have tried on various levels to increase the photosynthetic efficiency but no one has tried or demonstrated to replace the existing photosynthetic system.

### SUMMARY

The disclosure provides a recombinant microorganism or plant comprising a metabolic pathway for the synthesis of acetyl-CoA and isocitrate from C4 compounds using a pathway comprising an enzyme having malate thiokinase (MTK) activity, malyl-CoA lyase (MCL) activity and isocitrate lyase (ICL) activity. In one embodiment, the microorganism is a prokaryote or eukaryote. In another embodiment, the microorganism is yeast. In yet another embodiment, the microorganism is a prokaryote. In a further embodiment, the microorganism is derived from an E. coli microorganism. In yet a further embodiment of any of the foregoing the organism is engineered to express a malate thiokinase. In a further embodiment, the malate thiokinase is cloned from Methylococcus capsulatus. In yet another embodiment, the malate thiokinase comprises a heterodimer of sucC-2 and sucD-2 from Methylcoccus capsulatus. In yet another embodiment, the malate thiokinase comprises a sequence that is at least 40% to 100% identical to SEQ ID NO:2 and 4 and converts malate to malyl-coA. In another embodiment, a recombinant plant can comprise a polynucleotide encoding a malate thiokinase (mtkA) a sequence that is 40%-100% identical to SEQ ID NO:28. The polynucleotide can comprise a sequence that has a sequence as set forth in SEQ ID NO:27, operably linked to a 35S promoter or other suitable plant promoter. In another embodiment, a recombinant plant can comprise a polynucleotide encoding a malate thiokinase (mtkB) a sequence that is 40%-100% identical to SEQ ID NO:30. The polynucleotide can comprise a sequence that has a sequence as set forth in SEQ ID NO:29, operably linked to a 35S promoter or other suitable plant promoter. In a further embodiment of any of the foregoing the recombinant microorganism or plant is engineered to express a malyl-coA lyase. In a further embodiment, the malyl-coA lyase is cloned from Rhodobacter sphaeroides. In yet a further embodiment, the malyl-coA lyase comprises a mcl1 from Rhodobacter sphaeroides. In still yet a further embodiment, the malyl-coA lyase comprises a sequence that is at least 40% to 100% identical to SEQ ID NO:8 and converts malyl-coA to glyoxylate. In another embodiment of any of the foregoing the recombinant microorganism or plant is engineered to express or overexpress an isocitrate lyase. In a further embodiment, the isocitrate lyase is cloned from E. coli. In yet another embodiment, the isocitrate lyase comprises aceA from E. coli. In yet a further embodiment, the isocitrate lyase comprises a sequence that is at least 40% to 100% identical to SEQ ID NO:10 and converts glyoxylate and succinate to isocitrate. In further embodiments of any of the foregoing the microorganism or plant expresses or over expresses malate dehydrogenase. In yet another embodiment, the recombinant microorganism or plant of any of the foregoing embodiment, is engineered to heterologously expresses one or more of the following enzymes:

(a) a malate thiokinase;
(b) a malyl-coA lyase; and
(c) an isocitrate lyase.
In another embodiment, the microorganism or plant is further engineered to express or over express a malate dehydrogenase. In another embodiment, the microorganism or plant is further engineered to express or over express an aconitase. In yet another embodiment, the microorganism or plant is further engineered to express or over express an ATP citrate lyase. In another embodiment, the microorganism or plant further comprises one or more genes selected from the group consisting of atoB, hbd, crt, ter, and adhE2, and wherein the microorganism or plant produces 1-butanol. In another embodiment, the recombinant microorganism or plant comprises any of the foregoing pathways and further comprises one or more genes set forth in the figures for the production of ethanol, fatty acids and isoprenoids. In one embodiment, the microorganism or plant comprises a pathway for the production of acetyl-coA from C4 substrates as set forth in any of the foregoing embodiments coupled with a CO2 fixation pathway. In another embodiment, the recombinant microorganism or plant of any of the foregoing further comprises one or more knockouts selected from the group consisting of: Δicd, ΔgltA, ΔadhE, and Δack.

The disclosure provides a recombinant microorganism or plant that produces acetyl-CoA from C4 substrates/metabolites using an rGS pathway of FIG. 1, wherein the pathway is further extended to utilize acetyl-coA or pyruvate for the production of alcohols, fatty acids, isoprenoids and the like using pathways set forth in one or a combination of FIGS. 12a-f.

The disclosure also provides a method of making a desired metabolite comprising culturing any of the recombinant microorganisms or plants in the foregoing embodiment with a suitable substrate to produce the metabolite. The method further includes isolating the metabolite.

The disclosure also provides a transgenic plant or plant part comprising a Reverse Glyoxylate Shunt (rGS) pathway. The rGS pathway comprises aconitase, NADP-Malate dehydrogenase, fumarase, fumerase reductase, malate thiokinase, Malyl-CoA, Isocitrtae lyase, ATP-Citrate Lyase, Puruvate oxidoreductase, and pyruvate carboxylase, wherein the plant exhibits improved plant biomass compared to a wild-type plant. In some embodiments, the plant part is a cell, root, leaves, anther, flower, seed, stalk or petiole.

The disclosure also provides a method to improve photosynthetic efficiency by utilizing less ATP molecules and increasing the photosynthetic rates. In one embodiment, introducing the rGS pathway into an sbpase mutant results in better plant growth and attaining more plant height due to improved CO₂ fixation in plants.

The disclosure also provide transgenic plants comprising increased oil content compared to wild-type or parental plant. The disclosure also provides a method of improving an oil crop or biofuel crop comprising expression of rGS genes/pathway in the plant, wherein the plant comprises increased acetyl-co-A or increased flux of acetyl-CoA flux, and increased fatty acid content and composition and further comprises a beneficial trait when compared to a plant that lacks the expression of rGS genes. In one embodiment, the disclosure provides a seed produced by such a plant or a DNA-containing plant part of such a plant. In another embodiment, such a plant part is further defined as a cell, meristem, root, leaf, node, pistil, anther, flower, seed, embryo, stalk or petiole.

The disclosure also provides a method of producing plant biomass, the method comprising: (a) obtaining a plant exhibiting expression of an rGS pathway; (b) growing said plant under plant growth conditions to produce plant tissue from the plant; and (c) preparing biomass from said plant tissue. In one embodiment, said preparing biomass comprises harvesting said plant tissue. In another embodiment, such a method further comprises using the biomass for biofuel production.

The disclosure also provides a method of making a commodity product comprising: (a) obtaining a plant exhibiting expression of an rGS pathway, wherein the sugar content of the plant is increased when compared to a plant that lacks the expression of the rGS pathway; (b) growing the plant under plant growth conditions to produce plant tissue from the plant; and (c) preparing a commodity product from the plant tissue. In one embodiment, preparing the commodity product comprises harvesting the plant tissue. In another embodiment, the commodity product is selected from the group consisting of vegetable oil, ethanol, butanol, biodiesel, biogas, carbon fiber, animal feed, fatty acids, isoprenoids and fermentable biofuel feedstock.

The disclosure provides a recombinant plant having increased CO₂ utilization compared to a wild-type or parental plant, the recombinant plant engineered to express one or more enzyme having activity selected form the group consisting of malate thiokinase activity, malyl-CoA lyase activity and pyruvate:ferrodoxin oxidoreductase activity. In one embodiment, the plant exhibits increased biomass compared to a wild-type or parental plant. In a further embodiment, the plant has a mutant sbpase gene. In yet another embodiment, the plant comprises a reduced expression or activity of RuBisco. In another embodiment of any of the foregoing, the plant is a crop plant for biofuel, cereal or forage. In another embodiment of any of the foregoing, the plant is an Arabidopsis, canola or camelina crop plant. In another embodiment of any of the foregoing, the plant is a monocotyledonous plant. In another embodiment of any of the foregoing, the plant is a dicotyledonous plant. In another embodiment of any of the foregoing, the recombinant plant comprises elevated acetyl-CoA content or synthesis flux compared to a wild-type or parental plant. In another embodiment of any of the foregoing, the recombinant plant comprises elevated oil content compared to a wild-type or parental plant. In another embodiment of any of the foregoing, the plant expresses or over expresses enzymes selected from the group consisting of aconitase, NADP-malate dehydrogenase, fumarase, fumarate reductase, ATP-citrate lyase, pyrufate:ferrodoxin oxidoreductase, malate thiokinase, malyl-CoA lyase, isocitrate lyase, pyruvate carboxylase and any combination thereof. In another embodiment of any of the foregoing, the plant comprises a genotype of acn, mdh, fumc, frd, acl, nifJ, mtkA, mtkB, mcl, icl, and pyc.

The disclosure also provides a plant part obtained from the recombinant plant of the disclosure. In one embodiment, the plant part is a protoplast, cell, meristem, root, pistil, anther, flower, seed, embryo, stalk or petiole.

The disclosure also provides a product produced from a recombinant plant of the disclosure.

The disclosure also provides a product produced from the plant part.

The disclosure provides a method for increasing carbon fixation and/or increasing biomass production in a plant, comprising: introducing into a plant, plant part, and/or plant cell one or more heterologous polynucleotides encoding polypeptides having the enzyme activity of aconitase, NADP-malate dehydrogenase, fumarase, fumarate reductase, ATP-citrate lyase, pyrufate:ferrodoxin oxidoreductase, malate thiokinase, malyl-CoA lyase, isocitrate lyase, and pyruvate carboxylase to produce a stably transformed plant, plant part, and/or plant cell expressing said one or more heterologous polynucleotides. In one embodiment, the one or more heterologous polynucleotides are introduced into a nucleus and/or a chloroplast of said plant, plant part, and/or plant cell. In another embodiment of any of the foregoing, one or more of said polypeptides are operably linked to an amino acid sequence that targets said polypeptides to the chloroplast.

The disclosure also provides a stably transformed plant, plant part or plant cell produced by the method described above.

The disclosure also provides a stably transformed plant, plant part or plant cell comprising one or more heterologous polynucleotides encoding polypeptides having the enzyme activity of aconitase, NADP-malate dehydrogenase, fumarase, fumarate reductase, ATP-citrate lyase, pyrufate:ferrodoxin oxidoreductase, malate thiokinase, malyl-CoA lyase, isocitrate lyase, and pyruvate carboxylase.

The disclosure also provides a seed of the stably transformed plant of the disclosure, the seed comprises in its genome the one or more heterologous polynucleotides encoding polypeptides having the enzyme activity of aconitase, NADP-malate dehydrogenase, fumarase, fumarate reductase, ATP-citrate lyase, pyrufate:ferrodoxin oxidoreductase, malate thiokinase, malyl-CoA lyase, isocitrate lyase, and pyruvate carboxylase.

The disclosure also provides a product produced from the stably transformed plant, plant part or plant cell.

The disclosure also provides a product produced from the stably transformed seed.

In any of the foregoing product embodiment, the product can be a food, drink, animal feed, fiber, oil, pharmaceutical and/or biofuel.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1 shows the glyoxylate cycle in the context of E. coli central metabolism. The native glyoxylate cycle, as described by Kornberg and Krebs, is shown as well as the reverse glyoxylate cycle. ACN and MDH are known to be natively reversible. MS and CS are not easily reversible, but ATP-driven enzymes can accomplish the reverse reactions. CS=citrate synthase, ACN=aconitase, ICL=isocitrate lyase, MS=malate synthase, MDH=malate dehydrogenase, ACL=ATP-citrate lyase, MTK=malate thiokinase, MCL=malyl-CoA lyase.

FIG. 2 shows the genetic context used for testing reversibility of glyoxylate shunt enzymes. Genes prpC and gltA were deleted to construct the glutamate auxotroph strain that was used to test the reversibility of the glyoxylate shunt in vivo. Black lines show the native E. coli metabolism leading to glutamate biosynthesis. ‘X’ denotes a gene knockout. The horizontal pathway depicted in the figure shows the genes that were tested using this design. Open block arrows indicate carbon sources supplied in the growth medium.

FIG. 3A-B shows the reversibility of native glyoxylate shunt enzymes. (A) Versions of Glu⁻ strain overexpressing combinations of native MS and ICL genes were tested for their ability to grow on glucose minimal medium with the additives indicated beneath each plate. The strains tested expressed the malate transporter Bs dctA and (1) no additional genes; (2) Ec aceA; (3) Ec aceA+Ec aceB; (4) Ec aceA+Ec glcB. Images were scanned after 4 days of incubation at 37° C. See Table 1 for strains' detailed genotypes. (B) Enzyme activity of purified AceA was tested in vitro. Commercial isocitrate dehydrogenase was used in excess in this coupled assay.

FIG. 4A-B shows the reversal of the glyoxylate shunt with heterologous genes. (A) MTK enzyme activity of M. capsulatus sucCD-2 was tested in vitro using lysate from E. coli cells expressing Mc SucCD-2. Purified R. sphaeroides Mcl1 was used in excess in this coupled assay. (B) Versions of Glu⁻ strain overexpressing combinations of heterologous MTK and MCL genes and native ICL were tested for their ability to grow on glucose minimal medium with the additives indicated beneath each plate. The strains tested expressed the malate transporter Bs dctA and (5) R. sphaeroides mcl1, M. capsulatus sucCD-2; (6) Ec aceA, Rs mcl1, Mc sucCD-2; (7) Ec aceA, Rs mcl1; (8) Ec aceA, Mc sucCD-2. Images were scanned after 4 days of incubation at 37° C. See Table 1 for strains' detailed genotypes.

FIG. 5 shows genetic context used for testing ability of rGC genes to produce oxaloacetate. This diagram represents the aspartate auxotroph selection strain (Asp⁻) used to test the reversibility of the extended glyoxylate shunt pathway in vivo. The native E. coli metabolism is shown. ‘X’ indicates that the reaction has been interrupted by gene knockouts. Also shows is the successful strategy to reverse glyoxylate shunt and complement aspartate auxotrophy, including Citrate to Oxaloacetated by Acl, citrate-isocitated conversion by acnAB, glyoxylate and isocitrate conversion by aceA, isocitrate to succinate, malate to malyl-CoA by Mtk and malyl-CoA to glyoxilate by Mcl. Note that the gltA and citDEF reactions were also individually tested for OAA formation from citrate (see FIG. 6). Open block arrows indicate carbon sources supplied in the growth medium.

FIG. 6A-C shows the activity of pathways from citrate to OAA. (A) Versions of Asp⁻ expressing the citrate transporter citA from S. enterica were grown on glucose minimal medium with citrate to test three OAA production pathways: (9) none overexpressed, CL knockout; (10) Ec gltA overexpression, CL knockout; (11) none overexpressed, native expression of CL; (12) overexpression of C. tepidum aclAB, CL knockout. Plates were scanned after 2 days of incubation at 37° C. (B) Enzyme activity of purified ACL was tested in vitro. Commercial malate dehydrogenase was used in excess in this coupled assay. (C) Optimization of isocitrate branchpoint. The effect of icd deletion and Ec acnA or Ec acnB overexpression were tested in combination (Strains 13-18, see graph inset) in the Asp⁻ strain expressing Ec aceA. Growth was tested in liquid minimal glucose medium supplemented with glyoxylate and succinate.

FIG. 7A-B shows a pathway from malate to OAA. (A) Growth of the optimized Asp⁻ strain on minimal medium supplemented with glucose and 10 mM of the supplement indicated below each plate. In addition to expressing the malate transporter Bs dctA, strain (19) expressed Mc sucCD-2, Rs mcl1, Ec aceA, and Ct aclAB. Negative control strains do not overexpress the following genes: (20) no aclAB; (21) no mcl1; (22) no acnA and aceA. Plates were scanned after 7 days of incubation at 37° C. See Table 1 for strains' detailed genotypes. (B) Growth rates of strain (19) (triangles) and (21) (squares) were compared in liquid glucose minimal medium supplemented with aspartate (short-dashed lines); malate and succinate (solid lines); or without supplement (long-dashed lines).

FIG. 8A-C shows Bacillus subtilis DctA transporter allows malate uptake in E. coli Δppc mutant. M9 plates 2% Glucose 100 μM IPTG with (A) no supplements, or (B) supplemented with 20 mM malate, or (C) 20 mM succinate. Scanned after 1 day of incubation at 37° C. All strains are E. coli JW3928 (Δppc) expressing E. coli or Bacillus subtilis dctA gene on a plasmid (Δppc pEcDctA or Δppc pBsDctA, respectively. In main text Table 1, these plasmids are referred to as pSM13 and pSM22 respectively). Δppc strain cannot grow on minimal medium with glucose due to its lack of anaplerotic supply of OAA to replenish TCA cycle (A). It can grow on M9 glucose with a succinate supplement, due to its ability to specifically uptake this dicarboxylate (C). Malate, on the other hand, is transported very poorly in presence of glucose, as demonstrated by the slow growth with a malate supplement (B). Overexpression of the E. coli malate transporter dctA did not help malate uptake under these conditions. However, overexpression of the Bacillus subtilis dctA gene did allow for fast growth of the Δppc mutant on M9 supplemented with glucose and malate.

FIG. 9 shows bioprospection for in vitro activity of various MTK-homologous proteins expressed in E. coli. Labels on the x-axis refer to the organism the genes have been cloned from. Rpome: Ruegeria pomeroyi; Cauri: Chloroflexus auriantacus; Hmari: Haloarcula marismortui ATCC 43049; Iloih: Idiomarina loihiensis L2TR; Kpneu: Klebsiella pneumoniae 342; Mcaps: Methylococcus capsulatus str. Bath; Mflag: Methylobacillus flagellatus KT; Psyri: Pseudomonas syringae pv. syringae; Saure: Staphylococcus aureus subsp. aureus USA300_TCH959; Sente: Salmonella enterica subsp. enterica serovar Typhi str. CT18; Rspha: Rhodobacter sphaeroides ATCC 17025; Bsubt: Bacillus subtilis; Patla: Pseudoalteromonas atlantica T6c; Cpsyc: Colwellia psychrerythraea 34H; Reutr: Ralstonia eutropha; E coli wt: Escherichia coli K-12 substr. MG1655; E coli x/y/z/xy/xz/yz: Escherichia coli K-12 substr. MG1655 sucCD genes carrying the mutations x and/or y and/or z that were tested for altering substrate specificity towards malate (see FIG. 10).

FIG. 10A-B shows protein alignment of MtkA/sucC and MtkB/SucD sequences. Dark bars below indicate residues around the active site; light bars indicate mutations tested on E. coli SucCD protein. G320A and V323N mutation in SucC are referred as the mutation “x”, P125A and T158A in SucD are referred as mutation “y” and “z”, respectively. Me: Methylobacterium extorquens; Rp: Ruegeria pomeroyi; Re: Ralstonia eutropha; Sa: Salmonella enterica; Ec: Escherichia coli. Alignment generated on Geneious software (Biomatters; Drummond A J, 2011) (A) mtkA(Me)=SEQ ID NO:50; mtkA(Rp)=SEQ ID NO:52; sucC(Re)=SEQ ID NO:54; sucC(Cc)=SEQ ID NO:55; sucC(Ec)=SEQ ID NO:57. (B) mtkB(Me)=SEQ ID NO:59; mtkB(Rp)=SEQ ID NO:61; sucD(Re)=SEQ ID NO:63; sucD(Sa)=SEQ ID NO:65; sucD(Ec)=SEQ ID NO:67.

FIG. 11 shows primer used in MtkAB homolog genes cloning and mutagenesisi. Bold indicate the overalp with the vector; lower case indicates themismatches in the site directed mutagenesis primers (SEQ ID NOs:68-106).

FIG. 12A-D shows pathways that can be extend from the rGS production of acetyl-CoA. (A) shows an extension of the rGS pathway of the disclosure to include carbon fixation (Pyruvate:ferredoxin oxidoreductase (pyruvate+2 oxidized ferredoxin+coenzyme A<=>acetyl-CoA+CO₂+2 reduced ferredoxin+H+) such as ydbK from Escherichia coli str. K-12 substr. MG1655, protein accession number: NP_415896.1, Gene ID: 946587 or homologous genes made up of either 1, 2 or 4 subunits; and Pyruvate carboxylase (pyruvate+bicarbonate+ATP <=>oxaloacetate+ADP+phosphate+H+) such as pycA from Bacillus subtilis subsp. subtilis str. 168, protein accession number: NP_389369.1, Gene ID: 935920 or homologous genes; or Pyruvate kinase (pyruvate+ATP <=>phosphoenolpyruvate+ADP+H+) such as pykF from Escherichia coli str. K-12 substr. MG1655, protein accession number: NP_416191.1, Gene ID: 946179 or homologous genes; and Phosphoenolpyruvate carboxylase (oxaloacetate+phosphate<=>phosphoenolpyruvate+bicarbonate), such as ppc from Escherichia coli str. K-12 substr. MG1655, protein accession number: NP_418391.1, Gene ID: 948457 or homologous genes. (B) shows the production of ethanol (acetaldehyde dehydrogenase (EC Number: 1.2.1.10) and ethanol dehydrogenase (EC Number: 1.1.1.1) (this can be a bifunctional enzyme)). (C) shows the production of isoprenoids (ATOB: Acetoacetyl-CoA thiolase, EC Number: 2.3.1.9; HMGS: hydroxymethylglutaryl-CoA synthase, EC Number: 2.3.3.10; HMGR: hydroxymethylglutaryl-CoA reductase, EC Number: 1.1.1.34; MK: mevalonate kinase, EC Number: 2.7.1.36; PMK: phosphor-mevalonate kinase, EC Number: 2.7.4.2; MVD1: mevalonate pyrophosphate decarboxylase; EC Number: 4.1.1.33; and IDI: isopentenyl pyrophosphate isomerase, EC Number: 5.3.3.2). (D) shows the production of fatty acids (ACC: acetyl-CoA carboxylase; EC Number: 6.4.1.2; FabD, malonyl-CoA:ACP transacylase; EC Number: 2.3.1.39/2.3.1.85/2.3.1.86; FabH, β-keto-acyl-ACP synthase III; EC Number: 2.3.1.180; FabB, β-keto-acyl-ACP synthase I; EC Number: 2.3.1.41; FabG, β-keto-acyl-ACP reductase; EC Number: 1.1.1.100; FabZ, β-hydroxyacyl-ACP dehydratase; EC Number: 4.2.1.59; FabI, enoyl-acyl-ACP reductase; EC Number: 1.3.1.9; and TesA, acyl-ACP thioesterase; EC Number: 3.1.2.14). (E) shows a pathway for production of n-butanol from acetyl-CoA produced from rGS. (f) shows production of isopropanol from acetyl-coA produced from rGS.

FIG. 13 shows an rGS pathway for use in plants.

FIG. 14 shows schematics of promoter-gene-termination arrangements that were integrated into the rGS pathway for plants.

FIG. 15 shows schematics of two binary vectors carrying the full rGS pathway as shown in FIG. 32.

FIG. 16 shows the insertion sites for T-DNA insertion lines sbpase and shows the affected genomic region for T-DNA insertion line sbpase.

FIG. 17 shows expression of rGS genes in chloroplasts. Plants transformed with rGS genes-chloroplast specific transient peptide-GFP constructs showing rGS genes expression in the chloroplast.

FIG. 18 shows comparative aerial growth analysis of sbpase mutants. 80-d-old mutants of sbpase and complemented transformed lines of sbpase [SBPase (sbpase::rGS) was compared and complemented lines show significant improvement in the plant height and plant biomass over mutant.

FIG. 19 shows genotyping of the sbp::rgS lines for the presence of all rGS genes in the transgenome. Genotyping of sbp::rGS lines have confirmed the presence of all rGS genes (Aconitase, NADP-MDH, Fumarase, FRD, mTK, ICl, PyC, acl and NifJ/POR) in the transgenome.

FIG. 20 shows comparative aerial growth analysis of WT and rGS::WT transgenic lines; 60-d-old WT-Col-0 plants and transgenic lines [WT::rGS] were compared and complemented lines rGS3 and rGS5 showed 22 and 27% significant improvement in the plant biomass (Average of n=5). Statistically significant difference t-test (P<0.05).

### DETAILED DESCRIPTION

As used herein and in the appended claims, the singular forms “a,” “and,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventors are not entitled to antedate such disclosure by virtue of prior disclosure.

The disclosure provide recombinant microorganisms and plants comprising a reverse glyoxylate shunt (rGS) that converts C₄ carboxylates into two molecules of acetyl-CoA without loss of CO₂. As an exemplary microorganism, E. coli was used to engineer such a pathway to convert malate and succinate to oxaloacetate and two molecules of acetyl-CoA. In another embodiment, an exemplary plant, Arabidopsis, was engineered with a rGS pathway. ATP-coupled heterologous enzymes were used at the thermodynamically unfavorable steps to drive the pathway in the desired direction. This synthetic pathway in essence reverses the glyoxylate shunt at the expense of ATP. When integrated with central metabolism, this pathway can increase the carbon yield of acetate and biofuels from many carbon sources in heterotrophic microorganisms, and provides a basis of novel carbon fixation cycles. The disclosure provides methods and compositions (including cell free systems and recombinant organisms).

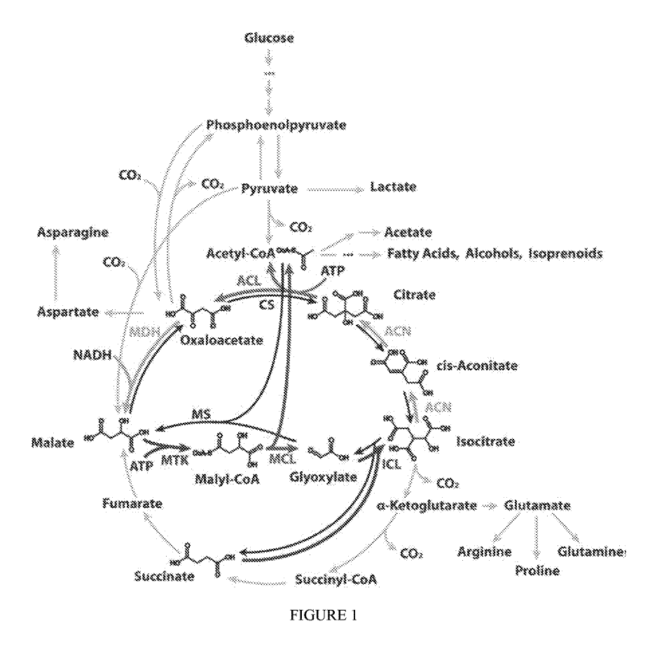

The tricarboxylic acid (TCA) cycle, in addition to generating energy and reducing power for cellular metabolism, provides intermediates that are essential precursors for numerous cellular building blocks. With each turn of the TCA cycle, one molecule of acetyl-CoA (C₂) is converted into free CoA, 2 molecules of CO₂, energy in the form of ATP, reducing equivalents in the form NAD(P)H, and water. The glyoxylate shunt, first described by Kornberg and Krebs in 1957 avoids the two decarboxylation steps of the TCA cycle, therefore allowing acetyl-CoA to be converted to TCA cycle intermediates without carbon loss (see, e.g., FIG. 1A, black line). This shunt is a feature of the glyoxylate cycle, which allows cells to grow on C₂ compounds such as acetate or fat-derived acetyl-CoA when carbohydrates are limited. The glyoxylate shunt involves two enzymes, isocitrate lyase (ICL) and malate synthase (MS), which convert isocitrate and acetyl-CoA to malate and succinate. While most central metabolic processes such as glycolysis, the TCA cycle, and β-oxidation of fatty acids, have counter-processes in the anabolic direction (gluconeogenesis, reductive TCA cycle, and fatty acid synthesis, respectively), the glyoxylate shunt has only been found to run in the acetyl-CoA assimilating, but not in the acetyl-CoA producing direction. As a result of this irreversibility, the most common sugars can only be metabolized to acetyl-CoA via decarboxylation of the three-carbon molecule pyruvate. This limitation creates a major loss of carbon in the utilization of carbohydrates by heterotrophic organisms for the synthesis of acetyl-CoA, a precursor to alcohols, fatty acids, isoprenoids and other useful bioenergy compounds. A synthetic pathway built upon a reverse version of the glyoxylate shunt, as described herein, provides a method of directly splitting a C₄ TCA intermediate into two acetyl-CoA molecules (FIG. 1). Since no reverse glyoxylate shunt (rGS) is known in nature, a synthetic rGS was designed, and to exemplify the pathway, incorporated into E. coli (FIG. 1, (MTK), (MCL), (ICL)). The reverse shunt was extended by introducing additional steps to convert isocitrate into acetyl-CoA and oxaloacetate (OAA) (FIG. 1 (CAN)), thereby constructing a pathway that allows for conversion of two C₄ molecules into one C₄ and two C₂ molecules. Genetic testing was performed to determine activity of individual steps in the pathway as well as the combined activity of the pathway from malate and succinate to oxaloacetate and two acetyl-CoA.

The pathway of the disclosure was developed using thermodynamic principles to engineer a pathway in a naturally unfavorable direction, utilizing ATP hydrolysis to drive key steps. Genetic selection were used to demonstrate activity of each step of the pathway individually and in combination. Metabolic engineering of native genes was required to direct flux in the desired direction. Using this general process the disclosure provides a novel pathway to the toolkit of metabolic engineers that allows for conversion of C₄ carboxylic acids to acetyl-CoA without carbon loss as CO₂.

There are a number of uses for this pathway based on rGS. For example, extension of the pathway by addition of malate dehydrogenase (MDH) would connect OAA to malate and allow for malate to cycle while converting succinate to acetyl-CoA. Separately, to convert malate to succinate and integrate the pathway described here with central metabolism, two additional enzymes (not formally involved in the glyoxylate shunt) are used: a fumarase and a fumarate reductase. E. coli encodes three fumarases, of which at least one is expressed during either aerobic or anaerobic conditions. Fumarate reductase (Frd) is generally only expressed anaerobically, and may need to be deregulated for full pathway integration. Deregulated Frd mutants have been previously found in selections for aerobic growth in succinate dehydrogenase null strains. Various fumarate reductases are known in the art.

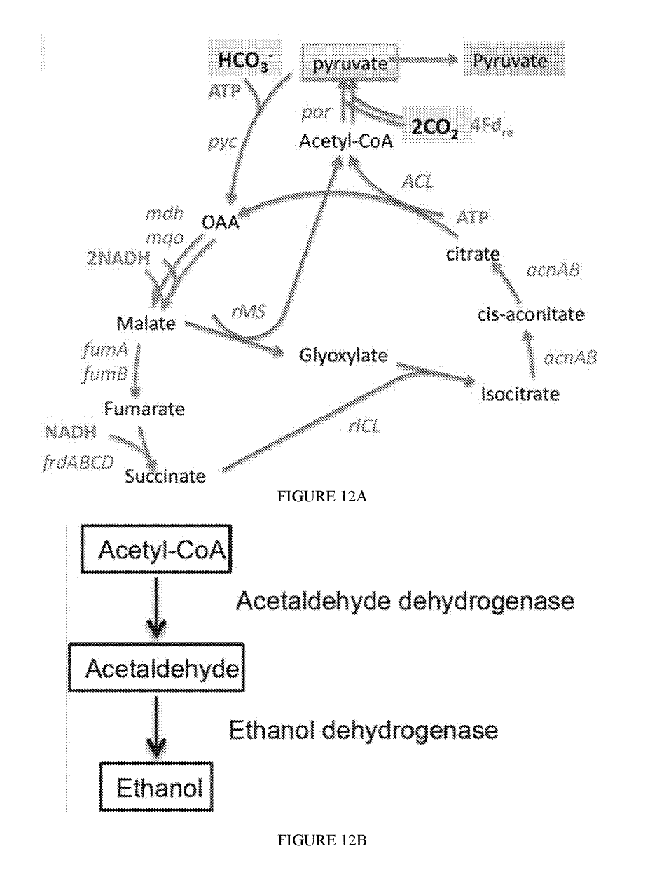

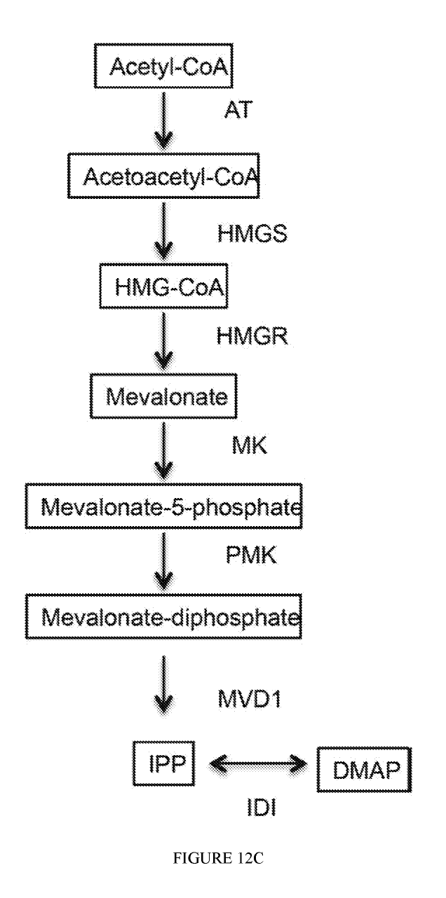

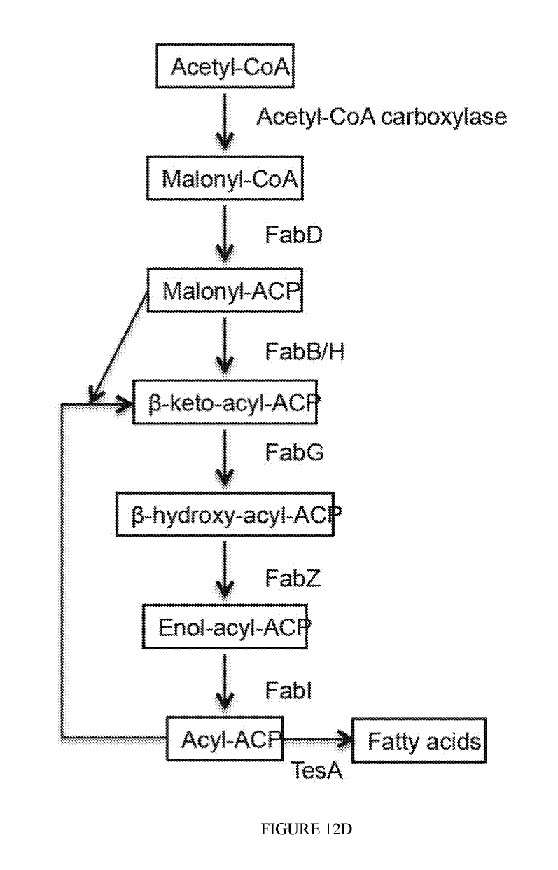

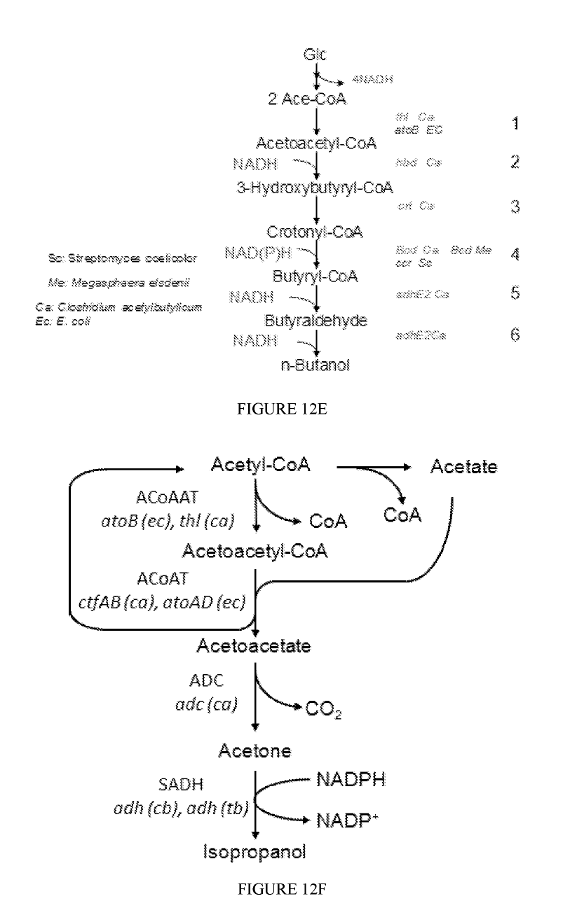

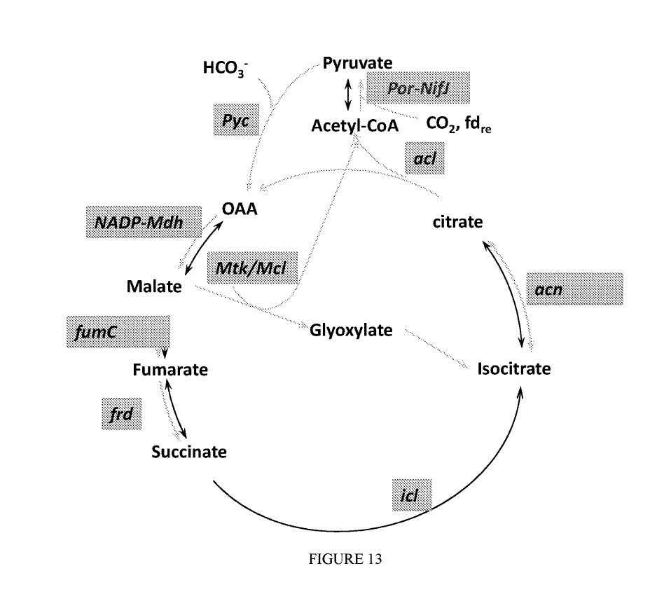

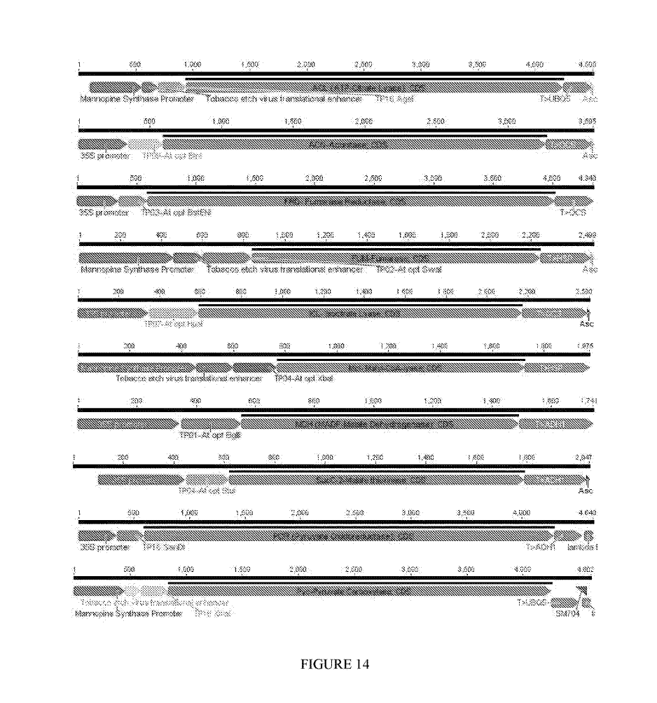

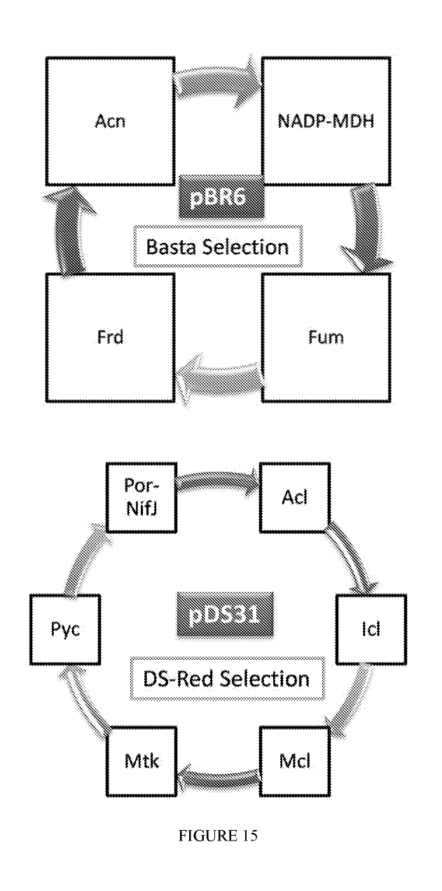

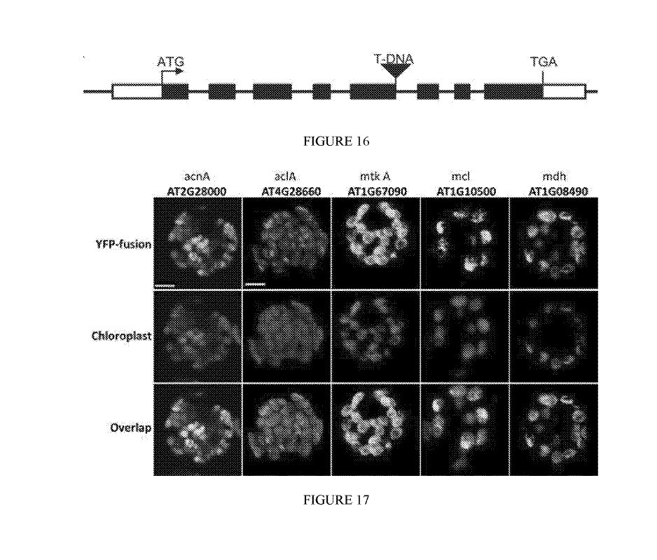

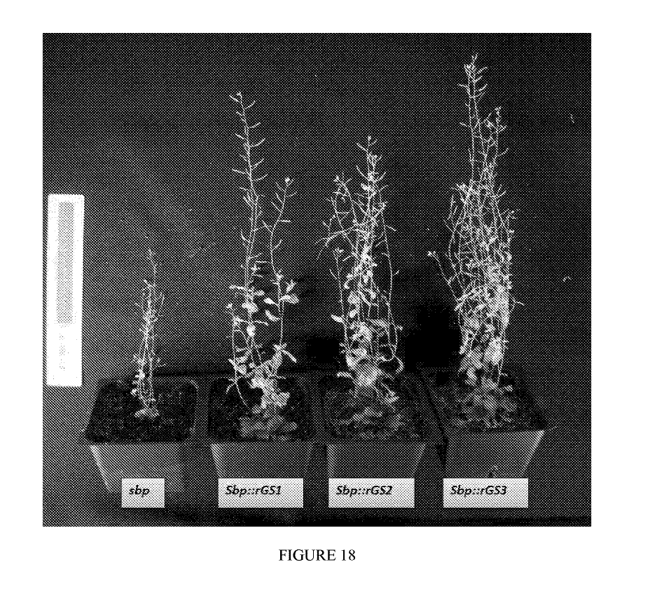

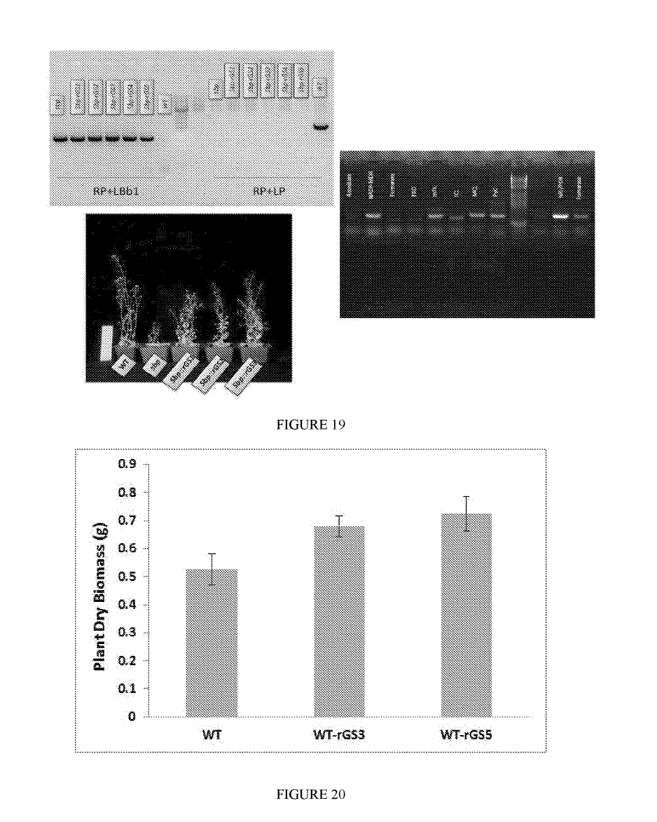

If integrated with central metabolism, for example via the native E. coli phosphoenolpyruvate carboxylase, such a pathway could theoretically allow for the conversion of one mole of glucose to 3 moles of acetyl-CoA, thus achieving a 50% yield increase over glycolysis. This yield increase can be channeled into industrially relevant compounds such as isoprenoids, fatty acids or long chain alcohols (see FIG. 1 and FIGS. 12A-F). The rGS pathway also allows conversion of a number of amino acids to acetyl-CoA at higher carbon yields than other known pathways. Protein-to-biofuel conversion has been of interest and would benefit from this pathway. Finally a CO₂ fixation cycle could be built upon the pathway described here. Addition of one enzyme to convert acetyl-CoA into pyruvate (e.g., pyruvate ferredoxin oxidoreductase) would close the linear CO₂ fixation pathway into a cycle and can allow growth with CO₂ as the sole carbon source (FIG. 13), in combination with a source of reducing power. In the experiments, ATP was provided by metabolism of glucose.

In the case of growth on CO₂, ATP could be provided from oxidation of an inorganic electron source such as H₂. The disclosure shows that with the introduction of 3 foreign enzymes, appropriate metabolic tuning, the reverse glyoxylate shunt pathway operates in vivo in E. coli and can be comparably modified into other organisms including, e.g., yeast and plants.

It should be recognized that the disclosure describes the pathway in various embodiments and is schematically depicted in FIG. 1. It will be further recognized that once Acetyl-CoA is produced the molecule can be further metabolized using pathways described for the production of Acetate, fatty acids, isoprenoids and other chemicals and biofuels (see, e.g., International application publication WO 2008/098227; WO 2008/124523; WO/2009/049274; WO 2010/071851; WO 2010/045629; WO 2011/037598; WO 2011/057288; WO 2011/088425; WO 2012/099934; WO 2012/135731; WO 2013/123454; WO 2013/126855, all of which are incorporated herein by references including all sequences).

In the pathways shown (in FIG. 1), Malate, Malyl-CoA, succinate and other C4 molecules can be used as the input molecule. The pathway uses investment of 4 carbon molecules such as, for example, malate, malyl-coA and succinate, which are split and recombined to produce acetyl-CoA without loss of CO₂. rGS utilizes 3 basic reactions and corresponding enzymes. One reaction is the conversion of malate to malyl-CoA. An enzyme useful for this reaction is malate thiokinase (MTK). MTK is typically found as a heterodimer of two polypeptides: (i) sucC-2 and SucD-2 (or homologs thereof). Another reaction is the conversion of malyl-CoA to glyoxylate and acetyl-CoA. An enzyme useful for this reaction is malyl-CoA lyase (MCL). MCLs useful in the disclosure can be derived from Rhodobacter sphaeroides mcl1 Citrate (Pro-3S)-lyase. The third reaction is the conversion of glyoxylate and succinate to form isocitrate. An enzyme useful for this reaction is isocitrate lyase (ICL). An ICL useful in the compositions and methods of the disclosure can be obtained from E. coli aceA gene.

The disclosure thus provides recombinant organisms comprising metabolically engineered biosynthetic pathways that comprise a non-CO₂ producing pathway for the production of acetyl-CoA from C4 molecules such as malate, malyl-CoA, and succinate. This pathway can be further extended to convert the acetyl-CoA to desirable products.

In one embodiment, the disclosure provides a recombinant microorganism or plant comprising elevated expression of at least one target enzyme as compared to a parental microorganism or plant or encodes an enzyme not found in the parental organism. In another or further embodiment, the microorganism or plant comprises a reduction, disruption or knockout of at least one gene encoding an enzyme that competes with a metabolite necessary for the production of a desired metabolite or which produces an unwanted product. The recombinant microorganism or plant produces at least one metabolite involved in a biosynthetic pathway for the production of, for example, acetyl-CoA. In general, the recombinant microorganism or plants comprises at least one recombinant metabolic pathway that comprises a target enzyme and may further include a reduction in activity or expression of an enzyme in a competitive biosynthetic pathway. The pathway acts to modify a substrate or metabolic intermediate in the production of, for example, acetyl-CoA. The target enzyme is encoded by, and expressed from, a polynucleotide derived from a suitable biological source. In some embodiments, the polynucleotide comprises a gene derived from a bacterial or yeast source and recombinantly engineered into the microorganism or plant of the disclosure. In another embodiment, the polynucleotide encoding the desired target enzyme is naturally occurring in the organism but is recombinantly engineered to be overexpressed compared to the naturally expression levels.

As used herein, an “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., unit measured by concentration or weight), or in terms of affinity or dissociation constants.

The term “biosynthetic pathway”, also referred to as “metabolic pathway”, refers to a set of anabolic or catabolic biochemical reactions for converting (transmuting) one chemical species into another. Gene products belong to the same “metabolic pathway” if they, in parallel or in series, act on the same substrate, produce the same product, or act on or produce a metabolic intermediate (i.e., metabolite) between the same substrate and metabolite end product. The disclosure provides recombinant microorganism or plant having a metabolically engineered pathway for the production of a desired product or intermediate.

Accordingly, metabolically “engineered” or “modified” microorganisms or plants are produced via the introduction of genetic material into a host or parental microorganism or plant of choice thereby modifying or altering the cellular physiology and biochemistry of the microorganism or plant to provide a recombinant metabolic pathway. Through the introduction of genetic material the parental microorganism or plant acquires new properties, e.g. the ability to produce a new, or greater quantities of, an intracellular metabolite. In an illustrative embodiment, the introduction of genetic material into a parental microorganism or plant results in a new or modified ability to produce acetyl-CoA through a non-CO₂ evolving pathway for optimal carbon utilization. The genetic material introduced into the parental microorganism or plant contains gene(s), or parts of gene(s), coding for one or more of the enzymes involved in a biosynthetic pathway for the production of acetyl-CoA, and may also include additional elements for the expression and/or regulation of expression of these genes, e.g. promoter sequences.

An engineered or modified microorganism or plant can also include in the alternative or in addition to the introduction of a genetic material into a host or parental microorganism, the reduction in expression, disruption, deletion or knocking out of a gene or polynucleotide to alter the cellular physiology and biochemistry of the microorganism or plant. Through the reduction, disruption or knocking out of a gene or polynucleotide the microorganism or plant acquires new or improved properties (e.g., the ability to produced a new or greater quantities of an intracellular metabolite, improve the flux of a metabolite down a desired pathway, and/or reduce the production of undesirable by-products).

An “enzyme” means any substance, typically composed wholly or largely of amino acids making up a protein or polypeptide that catalyzes or promotes, more or less specifically, one or more chemical or biochemical reactions.

The term “expression” with respect to a gene or polynucleotide refers to transcription of the gene or polynucleotide and, as appropriate, translation of the resulting mRNA transcript to a protein or polypeptide. Thus, as will be clear from the context, expression of a protein or polypeptide results from transcription and translation of the open reading frame.

As used herein, the term “metabolically engineered” or “metabolic engineering” involves rational pathway design and assembly of biosynthetic genes, genes associated with operons, and control elements of such polynucleotides, for the production of a desired metabolite, such as an acetyl-phosphate and/or acetyl-CoA, higher alcohols or other chemical, in a microorganism or plant. “Metabolically engineered” can further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway. Such metabolic engineering can includes selective modifications for co-factors for a particular pathway (e.g., NADH, NADPH, NAD⁺, NADP⁺, ATP, ADP, CoA and the like). A biosynthetic gene can be heterologous to the host microorganism or plant, either by virtue of being foreign to the host, or being modified by mutagenesis, recombination, and/or association with a heterologous expression control sequence in an endogenous host cell that results in higher expression compared to a wild-type organism. In one embodiment, where the polynucleotide is xenogenetic to the host organism, the polynucleotide can be codon optimized.

A “metabolite” refers to any substance produced by metabolism or a substance necessary for or taking part in a particular metabolic process that gives rise to a desired metabolite, chemical, alcohol or ketone. A metabolite can be an organic compound that is a starting material (e.g., succinate, malate, malyl-CoA, glyoxylate and the like (see, e.g., FIG. 1)), an intermediate in (e.g., acetyl-coA), or an end product (e.g., 1-butanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

A “native” or “wild-type” protein, enzyme, polynucleotide, gene, or cell, means a protein, enzyme, polynucleotide, gene, or cell that occurs in nature. As mentioned above, in some embodiment, a wild-type protein or polynucleotide may be linked to a heterologous promoter or regulatory elements and under such instances would become recombinantly expressed.

A “parental microorganism” or “parental plant” refers to a cell used to generate a recombinant microorganism or plant. The term “parental microorganism” or “parental plant” describes a cell that occurs in nature, i.e. a “wild-type” cell that has not been genetically modified. The term “parental microorganism” or “parental plant” also describes a cell that serves as the “parent” for further engineering. For example, a wild-type microorganism or plant can be genetically modified to express or over express a first target enzyme such as a malate thiokinase. This microorganism or plant can act as a parental microorganism or plant in the generation of a microorganism or plant modified to express or over-express a second target enzyme e.g., a malyl-CoA lyase. In turn, the microorganism or plant can be modified to express or over express a third enzyme, e.g., an isocitrate lyase, which can be further modified to express or over express a fourth target enzyme, e.g., aconitase, etc.

Accordingly, a parental microorganism or plant functions as a reference cell for successive genetic modification events. Each modification event can be accomplished by introducing one or more nucleic acid molecules in to the reference cell. The introduction of a polynucleotide facilitates the expression or over-expression of one or more target enzyme or the reduction or elimination of one or more target enzymes. It is understood that the term “facilitates” encompasses the activation of endogenous polynucleotides encoding a target enzyme through genetic modification of e.g., a promoter sequence in a parental microorganism or plant. It is further understood that the term “facilitates” encompasses the introduction of exogenous polynucleotides encoding a target enzyme in to a parental microorganism or plant.

A “protein” or “polypeptide”, which terms are used interchangeably herein, comprises one or more chains of chemical building blocks called amino acids that are linked together by chemical bonds called peptide bonds. A protein or polypeptide can function as an enzyme.

The term “polynucleotide,” “nucleic acid” or “recombinant nucleic acid” refers to polynucleotides such as deoxyribonucleic acid (DNA), and, where appropriate, ribonucleic acid (RNA).

Polynucleotides that encode enzymes useful for generating metabolites (e.g., enzymes such as malate thiokiase, malyl-coA lyase, isocitrate lyase, aconitase and the like) including homologs, variants, fragments, related fusion proteins, or functional equivalents thereof, are used in recombinant nucleic acid molecules that direct the expression of such polypeptides in appropriate host cells, such as bacterial or yeast cells. It is understood that the addition of sequences which do not alter the encoded activity of a nucleic acid molecule, such as the addition of a non-functional or non-coding sequence, is a conservative variation of the basic nucleic acid.

It is understood that a polynucleotide described above include “genes” and that the nucleic acid molecules described above include “vectors” or “plasmids.” For example, a polynucleotide encoding a malate thiokinase can comprise a sucC-2/sucD-2 gene or homolog thereof. Accordingly, the term “gene”, also called a “structural gene” refers to a polynucleotide that codes for a particular polypeptide comprising a sequence of amino acids, which comprise all or part of one or more proteins or enzymes, and may include regulatory (non-transcribed) DNA sequences, such as promoter region or expression control elements, which determine, for example, the conditions under which the gene is expressed. The transcribed region of the gene may include untranslated regions, including introns, 5′-untranslated region (UTR), and 3′-UTR, as well as the coding sequence.

Those of skill in the art will recognize that, due to the degenerate nature of the genetic code, a variety of codons differing in their nucleotide sequences can be used to encode a given amino acid. A particular polynucleotide or gene sequence encoding a biosynthetic enzyme or polypeptide described above are referenced herein merely to illustrate an embodiment of the disclosure, and the disclosure includes polynucleotides of any sequence that encode a polypeptide comprising the same amino acid sequence of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate certain embodiments of the disclosure. Such polypeptides may have from 1-50 (e.g., 1-10, 10-20, 20-30, 30-40 or 40-50) conservative amino acid substitutions as described herein while retaining their catalytic activity.

The disclosure provides polynucleotides in the form of recombinant DNA expression vectors or plasmids, as described in more detail elsewhere herein, that encode one or more target enzymes. Generally, such vectors can either replicate in the cytoplasm of the host microorganism or plant or integrate into the chromosomal DNA of the host microorganism or plant. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) form. The disclosure also includes non-naturally occurring cDNA molecules encoding the polypeptide useful in the disclosure. In addition, the disclosure includes modified sequences comprising a natural sequence wherein one or more nucleotides have been changed compared to a naturally occurring version. Such modified version can encode the same polypeptide sequence or modified polypeptide sequences with reference to the protein encoded by a naturally occurring sequences.

A polynucleotide of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated polynucleotide molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the polynucleotide by standard techniques, such as site-directed mutagenesis and PCR-mediated mutagenesis. In contrast to those positions where it may be desirable to make a non-conservative amino acid substitution, in some positions it is preferable to make conservative amino acid substitutions.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

The term “recombinant microorganism,” “recombinant plant” and “recombinant host cell” are used interchangeably herein and refer to microorganisms or plants that have been genetically modified to express or over-express endogenous polynucleotides, or to express non-endogenous sequences, such as those included in a vector. The polynucleotide generally encodes a target enzyme involved in a metabolic pathway for producing a desired metabolite as described above, but may also include protein factors necessary for regulation or activity or transcription. Accordingly, recombinant microorganisms or plants described herein have been genetically engineered to express or over-express target enzymes not previously expressed or over-expressed by a parental microorganism or plant. It is understood that the terms “recombinant microorganism,” “recombinant plant” and “recombinant host cell” refer not only to the particular recombinant microorganism or plant but to the progeny or potential progeny of such a microorganism or plant.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, but also intermediate and end product metabolites used in a pathway associated with a metabolically engineered microorganism or plant as described herein. With respect to the rGS pathway described herein, a starting material can be any suitable carbon source including, but not limited to, succinate, malate, malyl-CoA etc. Succinate, for example, can be converted to isocitrate or malate prior to entering the rGS pathway as set forth in FIG. 1.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

A “vector” generally refers to a polynucleotide that can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or plant or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433, which is incorporated herein by reference in its entirety), can also be used. For E. coli expression vectors, it is useful to include an E. coli origin of replication, such as from pUC, p1P, p1, and pBR.

Thus, recombinant expression vectors contain at least one expression system, which, in turn, is composed of at least a portion of a gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

The disclosure provides methods for the heterologous expression of one or more of the biosynthetic genes or polynucleotides involved in acetyl-phosphate synthesis, acetyl-CoA biosynthesis or other metabolites derived therefrom and recombinant DNA expression vectors useful in the method. Thus, included within the scope of the disclosure are recombinant expression vectors that include such nucleic acids.

Recombinant microorganisms and plants provided herein can express a plurality of target enzymes involved in pathways for the production of acetyl-CoA or other metabolites derived therefrom from a suitable carbon substrate such as, for example, malate, succinate and similar C4 molecules that can enter the pathway. The carbon source can be metabolized to, for example, an acetyl-CoA, which can be further metabolized to, e.g., fatty acids, alcohols and isoprenoids to name a few compounds. Sources of, for example, succinate, fumarate, oxaloacetate and malate are known.

The disclosure demonstrates that the expression or over expression of one or more heterologous polynucleotide or over-expression of one or more native polynucleotides encoding (i) a polypeptide that catalyzes the production of malyl-CoA from malate; (ii) a polypeptide that catalyzes the conversion of malyl-CoA to glyoxylate and acetyl-CoA; and (iii) a polypeptide the catalyzes the conversion of glyoxylate and succinate to isocitrate can utilize C4 carbon sources and produced acetyl-CoA without CO₂ loss. In other embodiment, additional polypeptides that convert isocitrate to cis-aconitate, cis-aconitate to citrate, citrate to oxaloacetate and acetyl-CoA, and oxaloacetate to malate can be incorporated to provide an effective cycle for acetyl-CoA production.

Microorganisms and plants provided herein are modified to produce metabolites in quantities and utilize carbon sources more effectively or utilize carbon sources not readily metabolized compared to a parental microorganism or plant. In particular, the recombinant microorganism or plant comprises a metabolic pathway for the production of acetyl-CoA using a C4 metabolite with conserved carbon or no CO₂ production. By “conserves carbon” is meant that the metabolic pathway that converts the C4 metabolite to acetyl-coA has a minimal or no loss of carbon from the starting C4 metabolite to the acetyl-coA. For example, in one embodiment, the recombinant microorganism or plant produces a stoichiometrically conserved amount of carbon product from the same number of carbons in the input carbon source (e.g., 1 succinate (a C4 metabolite) yields 2 acetyl-phosphate (two 2-carbon metabolites)).

Accordingly, the disclosure provides a recombinant microorganisms or plant that produce acetyl-CoA or other metabolites derived therefrom and includes the expression or elevated expression of target enzymes such as a malate thiokinase (e.g., sucC-2/sucD-2), a malyl-coA lyase (e.g., mcl1 citrate(pro-3S)-lyase), an isocitrate lyase (e.g., aceA), aconitase (e.g., acn), a malate dehydrogenase (e.g., Mdh), or any combination thereof, as compared to a parental microorganism or plant. The recombinant microorganism or plant may further includes a reduction in expression or activity, or a knockout of (i) an enzyme the converts citrate to oxaloacetate (e.g., citDEF), (ii) an enzyme that converts oxaloacetate and acetyl-CoA to citrate (e.g., gltA), (iii) an enzyme that converts phosphoenolpyruvate to oxaloacetate (e.g., ppc), (iv) an enzyme that converts oxaloacetate to malate (e.g., mdh/mqo), or any combination of (i)-(iv).

In some embodiments, where an acetyl-coA product is to be further metabolized, the recombinant microorganism or plant can express or over express a phosphotransacetylase (e.g., pta), and optionally may include expression or over expression of an acetate kinase. In addition, in these extended pathways the microorganism or plant may include a disruption, deletion or knockout of expression of an alcohol/acetaldehyde dehydrogenase that preferentially uses acetyl-coA as a substrate (e.g. adhE gene), as compared to a parental microorganism or plant. In some embodiments, further knockouts may include knockouts in a lactate dehydrogenase (e.g., ldh) and frdBC.

It will be recognized that organism that inherently have one or more (but not all) of the foregoing enzymes, which can be utilized as a parental organism. As described more fully below, a microorganism or plant of the disclosure comprising one or more recombinant genes encoding one or more enzymes above, and may further include additional enzymes that extend the acetyl-CoA product, which can then be extended to produce, for example, butanol, isobutanol, 2-pentanone and the like.

Accordingly, a recombinant microorganism or plant provided herein includes the elevated expression of at least one target enzyme, such as aceA or genes encoding the heterodimers sucC-2 and sucD-2. In other embodiments, a recombinant microorganism or plant can express a plurality of target enzymes involved in a pathway to produce acetyl-CoA or other metabolites derived therefrom as depicted in FIG. 1 and FIGS. 12A-F from a C4 carbon source such as succinate, malate and the like. In one embodiment, the recombinant microorganism or plant comprises expression of a heterologous or over expression of an endogenous enzyme selected from a malate thiokinase, a malyl-coA lyase, an isocitrate lyase and either or both of (i) malate dehydrogenase, and/or (ii) an aconitase.

As previously noted, the target enzymes described throughout this disclosure generally produce metabolites. In addition, the target enzymes described throughout this disclosure are encoded by polynucleotides. For example, a malate thiokinase can be encoded by sucC-2 and sucD-2 genes from Methylococcus capsulatus, polynucleotide or homolog thereof. The genes can be derived from any biologic source including Methylococcus capsulatus that provides a suitable nucleic acid sequence encoding a suitable enzyme having malate thiokinase activity.

Accordingly, in one embodiment, a recombinant microorganism or plant provided herein includes expression of a malate thiokinase (a heterodimer of sucC-2 and sucD2) as compared to a parental microorganism or plant. This expression may be combined with the expression or over-expression with other enzymes in the metabolic pathway for the production of acetyl-CoA or other metabolites derived therefrom as described herein above and below. The recombinant microorganism or plant produces a metabolite that includes malyl-CoA from malate, ATP and CoA. The malate thiokinase can be encoded by the genes sucC-2 and sucD2, polynucleotide or homolog thereof. The sucC-2 and sucD2 genes or polynucleotide can be derived from Methylococcus capsulatus.

In addition to the foregoing, the terms “malate thiokinase” or “sucC-2/sucD-2” refer to a heterodimeric protein that is capable of catalyzing the formation of malyl-CoA from malate, CoA and ATP, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:2, 4, 28, or 30. Additional homologs include: sequences having at least 50% homology (note that these sequences can be either annotated as succinyl-CoA synthetases, malate thiokinases or malate-CoA ligases): Methylobacterium extorquens AM1, MtkA: malate thiokinase, large subunit, Protein accession number: YP_002962851.1, (57% identity), converts malate to malyl-CoA; Ruegeria pomeroyi, malate-CoA ligase beta subunit, protein accession number: YP_166809.1, (58% identity), converts malate to malyl-CoA; Staphylococcus aureus subsp. aureus USA300_TCH959, succinate-CoA ligase, beta subunit, Protein accession number: EES93003.1, (55% identity), converts malate to malyl-CoA. Homologs of the sucD-2 sequence with at least 50% homology are (note that these sequences can be either annotated as succinyl-CoA synthetases or malate thiokinases): Methylobacterium extorquens AM1, MtkB: malate thiokinase, small subunit, protein accession number: YP_002962852.1 (58% identity), converts malate to malyl-CoA; Ruegeria pomeroyi DSS-3, succinyl-CoA synthetase, alpha subunit, protein accession number: YP_165609.1 (53% identity), converts malate to malyl-CoA; and Staphylococcus aureus subsp. aureus USA300_TCH959, succinate-CoA synthetase, alpha subunit, Protein accession number: EES93004.1, (54% identity), converts malate to malyl-CoA. The sequences associated with the foregoing accession numbers are incorporated herein by reference.

In another embodiment, a recombinant microorganism or plant provided herein includes elevated expression of malate dehydrogenase (Mdh) as compared to a parental microorganism or plant. This expression may be combined with the expression or over-expression with other enzymes in the metabolic pathway for the production of acetyl-CoA or other metabolites derived therefrom as described herein above and below. The recombinant microorganism or plant produces a metabolite that includes malate from a substrate that includes oxaloacetate and NADH. The malate dehydrogenase can be encoded by an Mdh gene, polynucleotide or homolog thereof. The Mdh gene or polynucleotide can be derived from various microorganisms including E. coli.

In addition to the foregoing, the terms “malate dehydrogenase” or “Mdh” refer to proteins that are capable of catalyzing the formation of malate from oxaloacetate and NADH, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:6 or 34. Malate dehydrogenase (EC 1.1.1.37), is an enzyme which functions in both the forward and reverse direction. S. cerevisiae possesses three copies of malate dehydrogenase, MDH1 (McAlister-Henn and Thompson, J. Bacteriol. 169:5157-5166 (1987), MDH2 (Minard and McAlister-Henn, Mol. Cell. Biol. 11:370-380 (1991); Gibson and McAlister-Henn, J. Biol. Chem. 278:25628-25636 (2003)), and MDH3 (Steffan and McAlister-Henn, J. Biol. Chem. 267:24708-24715 (1992)), which localize to the mitochondrion, cytosol, and peroxisome, respectively. E. coli is known to have an active malate dehydrogenase encoded by mdh. Other homologs that can be used in the methods and compositions of the disclosure that have 50% or more identity to SEQ ID NO:6 include Komagataella pastoris GS115, Mitochondrial malate dehydrogenase, Protein accession number: XP_002491128.1, (50% identity), catalyzes interconversion of malate and oxaloacetate; Klebsiella pneumonia, malate dehydrogenase, Protein accession number: WP_004206230.1, (95% identity), catalyzes interconversion of malate and oxaloacetate; and Aspergillus terreus NIH2624, malate dehydrogenase, mitochondrial precursor, Protein accession number: XP_001215536.1, (51% identity), catalyzes interconversion of malate and oxaloacetate.

In another embodiment, a recombinant microorganism or plant provided herein includes elevated expression of malyl-coA lyase as compared to a parental microorganism or plant. This expression may be combined with the expression or over-expression with other enzymes in the metabolic pathway for the production of acetyl-CoA or other metabolites derived therefrom as described herein above and below. The recombinant microorganism or plant produces a metabolite that includes glyoxylate and acetyl-coA from a substrate that includes malyl-coA. The malyl-coA lyase can be encoded by a mcl1 citrate (pro-3S)-lyase gene, polynucleotide or homolog thereof. The mcl1 gene or polynucleotide can be derived from various organisms including Rhodobacter sphaeroides. In another embodiment, the malyl-CoA lyase is derived from Methylobacterium extorquens. In another embodiment, in plants a polynucleotide encoding MCL is operably linked to a 35S or mannopine synthase promoter.

In addition to the foregoing, the terms “malyl-coA lyase” or “mcl1” or “MCL” refer to proteins that are capable of catalyzing the formation of glyoxylate and acetyl-coA from malyl-CoA, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:8 or 40. Examples of homologs of Rhodobacter sphaeroides mcl1 with at least 50% homology include, for example: Methylobacterium extorquens AM1, malyl-CoA lyase, mclA, Protein accession number: AAB58884.1, (58% identity), converts malyl-CoA into acetyl-CoA and glyoxylate; Ruegeria sp. TW15, malyl-CoA lyase, Protein accession number: WP_010437801, (57% identity), converts malyl-CoA into acetyl-CoA and glyoxylate; and Roseobacter denitrificans OCh 114, malyl-CoA lyase, Protein accession number: YP_684363, (57% identity), converts malyl-CoA into acetyl-CoA and glyoxylate. The sequences associated with the foregoing accession numbers are incorporated herein by reference.

In another embodiment, a recombinant microorganism or plant provided herein includes elevated expression of isocitrate lyase as compared to a parental microorganism or plant. This expression may be combined with the expression or over-expression with other enzymes in the metabolic pathway for the production of acetyl-CoA or other metabolites derived therefrom as described herein above and below. The recombinant microorganism or plant produces a metabolite that includes isocitrate from a substrate that includes succinate and glyoxylate. The isocitrate lyase can be encoded by an aceA gene, polynucleotide or homolog thereof. The aceA gene or polynucleotide can be derived from various organisms including E. coli and Ralstonia eutropha. In another embodiment, in plants a polynucleotide encoding an isocitrate lyase is operably linked to a 35S or mannopine synthase promoter.

In addition to the foregoing, the terms “isocitrate lyase” or “aceA” or “ICL” refer to proteins that are capable of catalyzing the formation of isocitrate from succinate and glyoxylate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:10 or 42. Additional homologs include: iclA of Ralstonia eutropha H16, Protein accession number: YP_726692.1 (70% identity), converts glyoxylate and succinate to isocitrate; aceA of Pseudomonas syringae pv. tomato str. DC3000I, Protein accession number: NP_793147.1, (73% identity), converts glyoxylate and succinate to isocitrate; and icl1 isocitrate lyase 1 from Rhizobium grahamii CCGE 502, Protein accession number: EPE99766.1, (59% identity), converts glyoxylate and succinate to isocitrate. The sequences associated with the foregoing accession numbers are incorporated herein by reference.

In another embodiment, a recombinant microorganism or plant provided herein includes elevated expression of aconitase (Acn) as compared to a parental microorganism or plant. This expression may be combined with the expression or over-expression with other enzymes in the metabolic pathway for the production of acetyl-CoA or other metabolites derived therefrom as described herein above and below. The recombinant microorganism or plant produces a metabolite that includes cis-aconitate from a substrate that includes isocitrate. The aconitase can be encoded by an Acn gene, polynucleotide or homolog thereof. The Acn gene or polynucleotide can be derived from various organisms including Arabidopsis thaliana.

In addition to the foregoing, the terms “aconitase” or “Acn” refer to proteins that are capable of catalyzing the formation of cis-aconitate from isocitrate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:32.

In another embodiment, a recombinant microorganism or plant provided herein includes elevated expression of fumarase (fumc) as compared to a parental microorganism or plant. This expression may be combined with the expression or over-expression with other enzymes in the metabolic pathway for the production of acetyl-CoA or other metabolites derived therefrom as described herein above and below. The recombinant microorganism or plant produces a metabolite that includes malate from a substrate that includes fumarate. The fumarase can be encoded by an fumc gene, polynucleotide or homolog thereof. The fumc gene or polynucleotide can be derived from various organisms including Synechocystis sp. PCC6803. In one embodiment, in plants the polynucleotide encoding a fumc is operably linked to a mannopine synthase promoter.

In addition to the foregoing, the terms “fumarase” or “fumc” refer to proteins that are capable of catalyzing the formation of malate from fumarate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:36.

In another embodiment, a recombinant microorganism or plant provided herein includes elevated expression of fumarate reductase (frd) as compared to a parental microorganism or plant. This expression may be combined with the expression or over-expression with other enzymes in the metabolic pathway for the production of acetyl-CoA or other metabolites derived therefrom as described herein above and below. The recombinant microorganism or plant produces a metabolite that includes succinate from a substrate that includes fumarate. The fumarate reductase can be encoded by an frd gene, polynucleotide or homolog thereof. The frd gene or polynucleotide can be derived from various organisms including Saccharomyces cerevisiae. In one embodiment, in plants the polynucleotide encoding a frd is operably linked to a 35S promoter.

In addition to the foregoing, the terms “fumarate reductase” or “frd” refer to proteins that are capable of catalyzing the formation of succinate from fumarate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:38.

In another embodiment, a recombinant microorganism or plant provided herein includes elevated expression of an ATP citrate lyase (ACL) as compared to a parental microorganism or plant. This expression may be combined with the expression or over-expression with other enzymes in the metabolic pathway for the production of acetyl-CoA or other metabolites derived therefrom as described herein above and below. The recombinant microorganism or plant produces a metabolite that includes oxaloacetate and acetyl-CoA from a substrate that includes citrate and ATP. The ATP citrate lyase can be encoded by an acl gene, polynucleotide or homolog thereof. The acl gene or polynucleotide can be derived from various organisms including Homo sapiens. In one embodiment, in plants the polynucleotide encoding an ACL is operably linked to a 35S or mannopine synthase promoter.

In addition to the foregoing, the terms “ATP citrate lyase” or “acl” refer to proteins that are capable of catalyzing the formation of oxaloacetate and acetyl-CoA, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:44.

In another embodiment, a recombinant microorganism or plant provided herein includes elevated expression of a pyruvate oxidoreductase (aka pyruvate ferrodoxin oxidoreductase) (nifJ gene; PFOR) as compared to a parental microorganism or plant. This expression may be combined with the expression or over-expression with other enzymes in the metabolic pathway for the production of acetyl-CoA or other metabolites derived therefrom as described herein above and below. The recombinant microorganism or plant produces a metabolite that includes pyruvate from a substrate that includes acetyl-CoA. The pyruvate oxidoreductase can be encoded by an nifJ gene, polynucleotide or homolog thereof. The nifJ gene or polynucleotide can be derived from various organisms including Synechocystis sp. PCC6803. In one embodiment, in plants the polynucleotide encoding an PFOR is operably linked to a 35S or mannopine synthase promoter.

In addition to the foregoing, the terms “pyruvate:ferrodoxin oxidoreductase” or “PFOR” refer to proteins that are capable of catalyzing the formation of pyruvate from acetyl-CoA, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:46.

In another embodiment, a recombinant microorganism or plant provided herein includes elevated expression of a pyruvate carboxylase (pyc) (EC 6.4.1.1) as compared to a parental microorganism or plant. This expression may be combined with the expression or over-expression with other enzymes in the metabolic pathway for the production of acetyl-CoA or other metabolites derived therefrom as described herein above and below. The recombinant microorganism or plant produces a metabolite that includes oxaloacetate from a substrate that includes pyruvate and ATP. The pyruvate carboxylase can be encoded by a pyc gene, polynucleotide or homolog thereof. The pyc gene or polynucleotide can be derived from various organisms including Lactococcus lactis. In one embodiment, in plants the polynucleotide encoding a pyc is operably linked to a 35S or mannopine synthase promoter.

In addition to the foregoing, the terms “pyruvate carboxylase” or “Pyc” refer to proteins that are capable of catalyzing the formation of oxaloacetate from pyruvate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:48.

As described herein and depicted in the figures the reverse glyoxylate shunt (rGS) can be combined with additional pathway enzymes that can metabolize acetyl-CoA (a product of rGS) to various chemicals including biofuels. Accordingly, one or more of the following enzymatic pathways may be further engineered into the recombinant microorganism or plant comprising an rGS pathway for the production of such metabolites (e.g., higher alcohols, fatty acids and isoprenoid).

Thus, in yet another embodiment, a recombinant microorganism or plant provided herein includes elevated expression of a crotonyl-CoA reductase as compared to a parental microorganism or plant. This expression may be combined with the expression or over-expression with other enzymes in the metabolic pathway for the production of n-butanol, isobutanol, butyryl-coA and/or acetone. The microorganism produces a metabolite that includes butyryl-CoA from a substrate that includes crotonyl-CoA. The crotonyl-CoA reductase can be encoded by a ccr gene, polynucleotide or homolog thereof. The ccr gene or polynucleotide can be derived from the genus Streptomyces.

Crotonyl-coA reductase catalyzes the reduction of crotonyl-CoA to butyryl-CoA. Depending upon the organism used a heterologous Crotonyl-coA reductase can be engineered for expression in the organism. Alternatively, a native Crotonyl-coA reductase can be overexpressed. Crotonyl-coA reductase is encoded in S. coelicolor by ccr. CCR homologs and variants are known. For examples, such homologs and variants include, for example, crotonyl CoA reductase (Streptomyces coelicolor A3(2)) gi|21224777|ref|NP_630556.1| (21224777); crotonyl CoA reductase (Streptomyces coelicolor A3(2)) gi|4154068|emb|CAA22721.1| (4154068); crotonyl-CoA reductase (Methylobacterium sp. 4-46) gi|168192678|gb|ACA14625.1| (168192678); crotonyl-CoA reductase (Dinoroseobacter shibae DFL 12) gi|159045393|ref|YP_001534187.1| (159045393); crotonyl-CoA reductase (Salinispora arenicola CNS-205) gi|159039522|ref|YP_001538775.1| (159039522); crotonyl-CoA reductase (Methylobacterium extorquens PA1) gi|163849740|ref|YP_001637783.1| (163849740); crotonyl-CoA reductase (Methylobacterium extorquens PA1) gi|163661345|gb|ABY28712.1| (163661345); crotonyl-CoA reductase (Burkholderia ambifaria AMMD) gi|115360962|ref|YP_778099.1| (115360962); crotonyl-CoA reductase (Parvibaculum lavamentivorans DS-1) gi|154252073|ref|YP_001412897.1| (154252073); Crotonyl-CoA reductase (Silicibacter sp. TM1040) gi|99078082|ref|YP_611340.1| (99078082); crotonyl-CoA reductase (Xanthobacter autotrophicus Py2) gi|154245143|ref|YP_001416101.1| (154245143); crotonyl-CoA reductase (Nocardioides sp. JS614) gi|119716029|ref|YP_922994.1| (119716029); crotonyl-CoA reductase (Nocardioides sp. JS614) gi|119536690|gb|ABL81307.1| (119536690); crotonyl-CoA reductase (Salinispora arenicola CNS-205) gi|157918357|gb|ABV99784.1| (157918357); crotonyl-CoA reductase (Dinoroseobacter shibae DFL 12) gi|157913153|gb|ABV94586.1| (157913153); crotonyl-CoA reductase (Burkholderia ambifaria AMMD) gi|115286290|gb|AB191765.1| (115286290); crotonyl-CoA reductase (Xanthobacter autotrophicus Py2) gi|154159228|gb|ABS66444.1| (154159228); crotonyl-CoA reductase (Parvibaculum lavamentivorans DS-1) gi|154156023|gb|ABS63240.1| (154156023); crotonyl-CoA reductase (Methylobacterium radiotolerans JCM 2831) gi|170654059|gb|ACB23114.1| (170654059); crotonyl-CoA reductase (Burkholderia graminis C4D1M) gi|170140183|gb|EDT08361.1| (170140183); crotonyl-CoA reductase (Methylobacterium sp. 4-46) gi|168198006|gb|ACA19953.1| (168198006); crotonyl-CoA reductase (Frankia sp. EAN1pec) gi|158315836|ref|YP_001508344.1| (158315836), each sequence associated with the accession number is incorporated herein by reference in its entirety.

Alternatively, or in addition to, the microorganism or plant provided herein includes elevated expression of a trans-2-hexenoyl-CoA reductase as compared to a parental microorganism or plant. The microorganism or plant produces a metabolite that includes butyryl-CoA from a substrate that includes crotonyl-CoA. The trans-2-hexenoyl-CoA reductase can also convert trans-2-hexenoyl-CoA to hexanoyl-CoA. The trans-2-hexenoyl-CoA reductase can be encoded by a ter gene, polynucleotide or homolog thereof. The ter gene or polynucleotide can be derived from the genus Euglena. The ter gene or polynucleotide can be derived from Treponema denticola. The enzyme from Euglena gracilis acts on crotonoyl-CoA and, more slowly, on trans-hex-2-enoyl-CoA and trans-oct-2-enoyl-CoA.

Trans-2-enoyl-CoA reductase or TER is a protein that is capable of catalyzing the conversion of crotonyl-CoA to butyryl-CoA, and trans-2-hexenoyl-CoA to hexanoyl-CoA. In certain embodiments, the recombinant microorganism or plant expresses a TER which catalyzes the same reaction as Bcd/EtfA/EtfB from Clostridia and other bacterial species. Mitochondrial TER from E. gracilis has been described, and many TER proteins and proteins with TER activity derived from a number of species have been identified forming a TER protein family (see, e.g., U.S. Pat. Appl. 2007/0022497 to Cirpus et al.; and Hoffmeister et al., J. Biol. Chem., 280:4329-4338, 2005, both of which are incorporated herein by reference in their entirety). A truncated cDNA of the E. gracilis gene has been functionally expressed in E. coli.

TER proteins can also be identified by generally well known bioinformatics methods, such as BLAST. Examples of TER proteins include, but are not limited to, TERs from species such as: Euglena spp. including, but not limited to, E. gracilis, Aeromonas spp. including, but not limited, to A. hydrophila, Psychromonas spp. including, but not limited to, P. ingrahamii, Photobacterium spp. including, but not limited, to P. profundum, Vibrio spp. including, but not limited, to V. angustum, V. cholerae, V. alginolyticus, V. parahaemolyticus, V. vulnificus, V. fischeri, V. splendidus, Shewanella spp. including, but not limited to, S. amazonensis, S. woodyi, S. frigidimarina, S. paeleana, S. baltica, S. denitrificans, Oceanospirillum spp., Xanthomonas spp. including, but not limited to, X. oryzae, X. campestris, Chromohalobacter spp. including, but not limited, to C. salexigens, Idiomarina spp. including, but not limited, to I. baltica, Pseudoalteromonas spp. including, but not limited to, P. atlantica, Alteromonas spp., Saccharophagus spp. including, but not limited to, S. degradans, S. marine gamma proteobacterium, S. alpha proteobacterium, Pseudomonas spp. including, but not limited to, P. aeruginosa, P. putida, P. fluorescens, Burkholderia spp. including, but not limited to, B. phytofirmans, B. cenocepacia, B. cepacia, B. ambifaria, B. vietnamensis, B. multivorans, B. dolosa, Methylbacillus spp. including, but not limited to, M. flageliatus, Stenotrophomonas spp. including, but not limited to, S. maltophilia, Congregibacter spp. including, but not limited to, C. litoralis, Serratia spp. including, but not limited to, S. proteamaculans, Marinomonas spp., Xytella spp. including, but not limited to, X. fastidiosa, Reinekea spp., Colweffia spp. including, but not limited to, C. psychrerythraea, Yersinia spp. including, but not limited to, Y. pestis, Y. pseudotuberculosis, Methylobacillus spp. including, but not limited to, M. flagellatus, Cytophaga spp. including, but not limited to, C. hutchinsonii, Flavobacterium spp. including, but not limited to, F. johnsoniae, Microscilla spp. including, but not limited to, M. marina, Polaribacter spp. including, but not limited to, P. irgensii, Clostridium spp. including, but not limited to, C. acetobutylicum, C. beijerenckii, C. cellulolyticum, Coxiella spp. including, but not limited to, C. burnetii.

In addition to the foregoing, the terms “trans-2-enoyl-CoA reductase” or “TER” refer to proteins that are capable of catalyzing the conversion of crotonyl-CoA to butyryl-CoA, or trans-2-hexenoyl-CoA to hexanoyl-CoA and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to either or both of the truncated E. gracilis TER or the full length A. hydrophila TER.

In yet another embodiment, a recombinant microorganism or plant provided herein includes elevated expression of a butyryl-CoA dehydrogenase as compared to a parental microorganism or plant. This expression may be combined with the expression or over-expression with other enzymes in the metabolic pathway for the production of 1-butanol, isobutanol, acetone, octanol, hexanol, 2-pentanone, and butyryl-coA as described herein above and below. The recombinant microorganism or plant produces a metabolite that includes butyryl-CoA from a substrate that includes crotonyl-CoA. The butyryl-CoA dehydrogenase can be encoded by a bcd gene, polynucleotide or homolog thereof. The bcd gene, polynucleotide can be derived from Clostridium acetobutylicum, Mycobacterium tuberculosis, or Megasphaera elsdenii.

In another embodiment, a recombinant microorganism or plant provided herein includes expression or elevated expression of an acetyl-CoA acetyltransferase as compared to a parental microorganism or plant. The microorganism or plant produces a metabolite that includes acetoacetyl-CoA from a substrate that includes acetyl-CoA. The acetyl-CoA acetyltransferase can be encoded by a thlA gene, polynucleotide or homolog thereof. The thlA gene or polynucleotide can be derived from the genus Clostridium.

Pyruvate-formate lyase (Formate acetyltransferase) is an enzyme that catalyzes the conversion of pyruvate to acetyl-coA and formate. It is induced by pfl-activating enzyme under anaerobic conditions by generation of an organic free radical and decreases significantly during phosphate limitation. Formate acetyltransferase is encoded in E. coli by pflB. PFLB homologs and variants are known. For examples, such homologs and variants include, for example, Formate acetyltransferase 1 (Pyruvate formate-lyase 1) gi|129879|sp|P09373.2|PFLB_ECOLI (129879); formate acetyltransferase 1 (Yersinia pestis CO92) gi|16121663|ref|NP_404976.1| (16121663); formate acetyltransferase 1 (Yersinia pseudotuberculosis IP 32953) gi|51595748|ref|YP_069939.1| (51595748); formate acetyltransferase 1 (Yersinia pestis biovar Microtus str. 91001) gi|45441037|ref|NP_992576.1| (45441037); formate acetyltransferase 1 (Yersinia pestis CO92) gi|115347142|emb|CAL20035.1| (115347142); formate acetyltransferase 1 (Yersinia pestis biovar Microtus str. 91001) gi|45435896|gb|AAS61453.1| (45435896); formate acetyltransferase 1 (Yersinia pseudotuberculosis IP 32953) gi|51589030|emb|CAH20648.1| (51589030); formate acetyltransferase 1 (Salmonella enterica subsp. enterica serovar Typhi str. CT18) gi|16759843|ref|NP_455460.1| (16759843); formate acetyltransferase 1 (Salmonella enterica subsp. enterica serovar Paratyphi A str. ATCC 9150) gi|56413977|ref|YP_151052.1| (56413977); formate acetyltransferase 1 (Salmonella enterica subsp. enterica serovar Typhi) gi|16502136|emb|CAD05373.1| (16502136); formate acetyltransferase 1 (Salmonella enterica subsp. enterica serovar Paratyphi A str. ATCC 9150) gi|56128234|gb|AAV77740.1| (56128234); formate acetyltransferase 1 (Shigella dysenteriae Sd197) gi|82777577|ref|YP_403926.1| (82777577); formate acetyltransferase 1 (Shigella flexneri 2a str. 2457T) gi|30062438|ref|NP_836609.1| (30062438); formate acetyltransferase 1 (Shigella flexneri 2a str. 2457T) gi|30040684|gb|AAP16415.1| (30040684); formate acetyltransferase 1 (Shigella flexneri 5 str. 8401) gi|110614459|gb|ABF03126.1| (110614459); formate acetyltransferase 1 (Shigella dysenteriae Sd197) gi|81241725|gb|ABB62435.1| (81241725); formate acetyltransferase 1 (Escherichia coli O157:H7 EDL933) gi|12514066|gb|AAG55388.1|AE005279_8(12514066); formate acetyltransferase 1 (Yersinia pestis KIM) gi|22126668|ref |NP_670091.1| (22126668); formate acetyltransferase 1 (Streptococcus agalactiae A909) gi|76787667|ref|YP_330335.1| (76787667); formate acetyltransferase 1 (Yersinia pestis KIM) gi|21959683 |gb|AAM86342.1|AE013882_3(21959683); formate acetyltransferase 1 (Streptococcus agalactiae A909) gi|76562724|gb|ABA45308.1| (76562724); formate acetyltransferase 1 (Yersinia enterocolitica subsp. enterocolitica 8081) gi|123441844|ref|YP_001005827.1| (123441844); formate acetyltransferase 1 (Shigella flexneri 5 str. 8401) gi|110804911|ref|YP_688431.1| (110804911); formate acetyltransferase 1 (Escherichia coli UTI89) gi|91210004|ref|YP_539990.1| (91210004); formate acetyltransferase 1 (Shigella boydii Sb227) gi|82544641|ref|YP_408588.1| (82544641); formate acetyltransferase 1 (Shigella sonnei Ss046) gi|74311459|ref|YP_309878.1| (74311459); formate acetyltransferase 1 (Klebsiella pneumoniae subsp. pneumoniae MGH 78578) gi|152969488|ref|YP_001334597.1| (152969488); formate acetyltransferase 1 (Salmonella enterica subsp. enterica serovar Typhi Ty2) gi|29142384|ref|NP_805726.1| (29142384) formate acetyltransferase 1 (Shigella flexneri 2a str. 301) gi|24112311|ref|NP_706821.1| (24112311); formate acetyltransferase 1 (Escherichia coli O157:H7 EDL933) gi|15800764|ref|NP_286778.1| (15800764); formate acetyltransferase 1 (Klebsiella pneumoniae subsp. pneumoniae MGH 78578) gi|150954337|gb|ABR76367.1| (150954337); formate acetyltransferase 1 (Yersinia pestis CA88-4125) gi|149366640|ref|ZP_01888674.1| (149366640); formate acetyltransferase 1 (Yersinia pestis CA88-4125) gi|149291014|gb|EDM41089.1| (149291014); formate acetyltransferase 1 (Yersinia enterocolitica subsp. enterocolitica 8081) gi|122088805|emb|CAL11611.1| (122088805); formate acetyltransferase 1 (Shigella sonnei Ss046) gi|73854936|gb|AAZ87643.1| (73854936); formate acetyltransferase 1 (Escherichia coli UTI89) gi|91071578|gb|ABE06459.1| (91071578); formate acetyltransferase 1 (Salmonella enterica subsp. enterica serovar Typhi Ty2) gi|29138014|gb|AAO69575.1| (29138014); formate acetyltransferase 1 (Shigella boydii Sb227) gi|81246052|gb|ABB66760.1| (81246052); formate acetyltransferase 1 (Shigella flexneri 2a str. 301) gi|24051169|gb|AAN42528.1| (24051169); formate acetyltransferase 1 (Escherichia coli O157:H7 str. Sakai) gi|13360445|dbj |BAB34409.1| (13360445); formate acetyltransferase 1 (Escherichia coli O157:H7 str. Sakai) gi|15830240|ref|NP_309013.1| (15830240); formate acetyltransferase I (pyruvate formate-lyase 1) (Photorhabdus luminescens subsp. laumondii TTO1) gi|36784986|emb|CAE13906.1| (36784986); formate acetyltransferase I (pyruvate formate-lyase 1) (Photorhabdus luminescens subsp. laumondii TTO1) gi|37525558|ref|NP_928902.1| (37525558); formate acetyltransferase (Staphylococcus aureus subsp. aureus Mu50) gi|14245993|dbj|BAB56388.1| (14245993); formate acetyltransferase (Staphylococcus aureus subsp. aureus Mu50) gi|15923216|ref|NP_370750.1| (15923216); Formate acetyltransferase (Pyruvate formate-lyase) gi|81706366|sp|Q7A7X6.1|PFLB_STAAN (81706366); Formate acetyltransferase (Pyruvate formate-lyase) gi|81782287|sp|Q99WZ7.1|PFLB_STAAM (81782287); Formate acetyltransferase (Pyruvate formate-lyase) gi|81704726|sp|Q7A1W9.1|PFLB_STAAW (81704726); formate acetyltransferase (Staphylococcus aureus subsp. aureus Mu3) gi|156720691|dbj|BAF77108.1| (156720691); formate acetyltransferase (Erwinia carotovora subsp. atroseptica SCRI1043) gi|50121521|ref|YP_050688.1| (50121521); formate acetyltransferase (Erwinia carotovora subsp. atroseptica SCRI1043) gi|49612047|emb|CAG75496.1| (49612047); formate acetyltransferase (Staphylococcus aureus subsp. aureus str. Newman) gi|150373174|dbj|BAF66434.1| (150373174); formate acetyltransferase (Shewanella oneidensis MR-1) gi|24374439|ref|NP_718482.1| (24374439); formate acetyltransferase (Shewanella oneidensis MR-1) gi|24349015|gb|AAN55926.1|AE015730_3(24349015); formate acetyltransferase (Actinobacillus pleuropneumoniae serovar 3 str. JL03) gi|165976461|ref|YP_001652054.1| (165976461); formate acetyltransferase (Actinobacillus pleuropneumoniae serovar 3 str. JL03) gi|165876562|gb|ABY69610.1| (165876562); formate acetyltransferase (Staphylococcus aureus subsp. aureus MW2) gi|21203365|dbj|BAB94066.1| (21203365); formate acetyltransferase (Staphylococcus aureus subsp. aureus N315) gi|13700141|dbj|BAB41440.1| (13700141); formate acetyltransferase (Staphylococcus aureus subsp. aureus str. Newman) gi|151220374|ref|YP_001331197.1| (151220374); formate acetyltransferase (Staphylococcus aureus subsp. aureus Mu3) gi|156978556|ref|YP_001440815.1| (156978556); formate acetyltransferase (Synechococcus sp. JA-2-3B′a (2-13)) gi|86607744|ref|YP_476506.1| (86607744); formate acetyltransferase (Synechococcus sp. JA-3-3Ab) gi|86605195|ref|YP_473958.1| (86605195); formate acetyltransferase (Streptococcus pneumoniae D39) gi|116517188|ref|YP_815928.1| (116517188); formate acetyltransferase (Synechococcus sp. JA-2-3B′a (2-13)) gi|86556286|gb|ABD01243.1| (86556286); formate acetyltransferase (Synechococcus sp. JA-3-3Ab) gi|86553737|gb|ABC98695.1| (86553737); formate acetyltransferase (Clostridium novyi NT) gi|118134908|gb|ABK61952.1| (118134908); formate acetyltransferase (Staphylococcus aureus subsp. aureus MRSA252) gi|49482458|ref|YP_039682.1| (49482458); and formate acetyltransferase (Staphylococcus aureus subsp. aureus MRSA252) gi|49240587|emb|CAG39244.1| (49240587), each sequence associated with the accession number is incorporated herein by reference in its entirety.

An acetoacetyl-coA thiolase (also sometimes referred to as an acetyl-coA acetyltransferase) catalyzes the production of acetoacetyl-coA from two molecules of acetyl-coA. Depending upon the organism used a heterologous acetoacetyl-coA thiolase (acetyl-coA acetyltransferase) can be engineered for expression in the organism. Alternatively a native acetoacetyl-coA thiolase (acetyl-coA acetyltransferase) can be overexpressed. Acetoacetyl-coA thiolase is encoded in E. coli by thl. Acetyl-coA acetyltransferase is encoded in C. acetobutylicum by atoB. THL and AtoB homologs and variants are known. For examples, such homologs and variants include, for example, acetyl-coa acetyltransferase (thiolase) (Streptomyces coelicolor A3(2)) gi|21224359|ref|NP_630138.1| (21224359); acetyl-coa acetyltransferase (thiolase) (Streptomyces coelicolor A3(2)) gi|3169041|emb|CAA19239.1| (3169041); Acetyl CoA acetyltransferase (thiolase) (Alcanivorax borkumensis SK2) gi|110834428|ref|YP_693287.1| (110834428); Acetyl CoA acetyltransferase (thiolase) (Alcanivorax borkumensis SK2) gi|110647539|emb|CAL17015.1| (110647539); acetyl CoA acetyltransferase (thiolase) (Saccharopolyspora erythraea NRRL 2338) gi|133915420|emb|CAM05533.1| (133915420); acetyl-coa acetyltransferase (thiolase) (Saccharopolyspora erythraea NRRL 2338) gi|134098403|ref|YP_001104064.1| (134098403); acetyl-coa acetyltransferase (thiolase) (Saccharopolyspora erythraea NRRL 2338) gi|133911026|emb|CAM01139.1| (133911026); acetyl-CoA acetyltransferase (thiolase) (Clostridium botulinum A str. ATCC 3502) gi|148290632|emb|CAL84761.1| (148290632); acetyl-CoA acetyltransferase (thiolase) (Pseudomonas aeruginosa UCBPP-PA14) gi|115586808|gb|ABJ12823.1| (115586808); acetyl-CoA acetyltransferase (thiolase) (Ralstonia metallidurans CH34) gi|93358270|gb|ABF12358.1| (93358270); acetyl-CoA acetyltransferase (thiolase) (Ralstonia metallidurans CH34) gi|93357190|gb|ABF11278.1| (93357190); acetyl-CoA acetyltransferase (thiolase) (Ralstonia metallidurans CH34) gi|93356587|gb|ABF10675.1| (93356587); acetyl-CoA acetyltransferase (thiolase) (Ralstonia eutropha JMP134) gi|72121949|gb|AAZ64135.1| (72121949); acetyl-CoA acetyltransferase (thiolase) (Ralstonia eutropha JMP134) gi|72121729|gb|AAZ63915.1| (72121729); acetyl-CoA acetyltransferase (thiolase) (Ralstonia eutropha JMP134) gi|72121320|gb|AAZ63506.1| (72121320); acetyl-CoA acetyltransferase (thiolase) (Ralstonia eutropha JMP134) gi|72121001|gb|AAZ63187.1| (72121001); acetyl-CoA acetyltransferase (thiolase) (Escherichia coli) gi|2764832|emb|CAA66099.1| (2764832), each sequence associated with the accession number is incorporated herein by reference in its entirety.

Butyryl-coA dehydrogenase is an enzyme in the protein pathway that catalyzes the reduction of crotonyl-CoA to butyryl-CoA. A butyryl-CoA dehydrogenase complex (Bcd/EtfAB) couples the reduction of crotonyl-CoA to butyryl-CoA with the reduction of ferredoxin. Depending upon the organism used a heterologous butyryl-CoA dehydrogenase can be engineered for expression in the organism. Alternatively, a native butyryl-CoA dehydrogenase can be overexpressed. Butyryl-coA dehydrogenase is encoded in C. acetobuylicum and M. elsdenii by bcd. BCD homologs and variants are known. For examples, such homologs and variants include, for example, butyryl-CoA dehydrogenase (Clostridium acetobutylicum ATCC 824) gi|15895968|ref|NP_349317.1| (15895968); Butyryl-CoA dehydrogenase (Clostridium acetobutylicum ATCC 824) gi|15025744|gb|AAK80657.1|AE007768_11(15025744); butyryl-CoA dehydrogenase (Clostridium botulinum A str. ATCC 3502) gi|148381147|ref|YP_001255688.1| (148381147); butyryl-CoA dehydrogenase (Clostridium botulinum A str. ATCC 3502) gi|148290631|emb|CAL84760.1| (148290631), each sequence associated with the accession number is incorporated herein by reference in its entirety. BCD can be expressed in combination with a flavoprotein electron transfer protein. Useful flavoprotein electron transfer protein subunits are expressed in C. acetobutylicum and M. elsdenii by a gene etfA and etfB (or the operon etfAB). ETFA, B, and AB homologs and variants are known. For examples, such homologs and variants include, for example, putative a-subunit of electron-transfer flavoprotein gi|1055221|gb|AAA95970.1| (1055221); putative b-subunit of electron-transfer flavoprotein gi|1055220|gb|AAA95969.1| (1055220), each sequence associated with the accession number is incorporated herein by reference in its entirety.

In yet other embodiment, in addition to any of the foregoing and combinations of the foregoing, additional genes/enzymes may be used to produce a desired product. For example, the following table provide enzymes that can be combined with the rGS pathway enzymes for the production of 1-butanol:

| | Exemplary | | |
|---|---|---|---|
| Enzyme | Gene(s) | 1-butanol | Exemplary Organism |
| Ethanol Dehydrogenase | adhE | − | E. coli |
| Lactate Dehydrogenase | ldhA | − | E. coli |
| Fumarate reductase | frdB, frdC, | − | E. coli |
| | or frdBC | | |
| Oxygen transcription | fnr | − | E. coli |
| regulator | | | |
| Phosphate | pta | − | E. coli |
| acetyltransferase | | | |
| Formate | pflB | − | E. coli |
| acetyltransferase | | | |
| acetyl-coA | atoB | + | C. acetobutylicum |
| acctyltransferase | | | |
| acetoacetyl-coA | thl, thlA, | + | E. coli, |
| thiolase | thlB | | C. acetobutylicum |
| 3-hydroxybutyryl-CoA | hbd | + | C. acetobutylicum |
| dehydrogenase | | | |
| crotonase | crt | + | C. acetobutylicum |
| butyryl-CoA | bcd | + | C. acetobutylicum, |
| dehydrogenase | | | M. elsdenii |
| electron transfer | etfAB | + | C. acetobutylicum, |
| flavoprotein | | | M. elsdenii |
| aldehyde/alcohol | adhE2 | + | C. acetobutylicum |
| dehydrogenase (butyral- | bdhA/bdhB | | |
| dehyde | aad | | |
| dehydrogenase/butanol | | | |
| dehydrogenase) | | | |
| crotonyl-coA reductase | ccr | + | S. coelicolor |
| trans-2-enoyl-CoA | Ter | + | T. denticola, |
| reductase | | | F. succinogenes |

* knockout or a reduction in expression are optional in the synthesis of the product, however, such knockouts increase various substrate intermediates and improve yield.

In addition, and as mentioned above, homologs of enzymes useful for generating metabolites are encompassed by the microorganisms, plants and methods provided herein. The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

In some instances “isozymes” can be used that carry out the same functional conversion/reaction, but which are so dissimilar in structure that they are typically determined to not be “homologous”. For example, tktB is an isozyme of tktA, talA is an isozyme of talB and rpiB is an isozyme of rpiA.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

The disclosure provides accession numbers for various genes, homologs and variants useful in the generation of recombinant microorganism or plant described herein. It is to be understood that homologs and variants described herein are exemplary and non-limiting. Additional homologs, variants and sequences are available to those of skill in the art using various databases including, for example, the National Center for Biotechnology Information (NCBI) access to which is available on the World-Wide-Web.

Culture conditions suitable for the growth and maintenance of a recombinant microorganism or plant provided herein are described in the Examples below. The skilled artisan will recognize that such conditions can be modified to accommodate the requirements of each microorganism or plant. Appropriate culture conditions useful in producing a acetyl-phosphate, acetyl-CoA or other metabolites derived therefrom including, but not limited to 1-butanol, n-hexanol, 2-pentanone and/or octanol products comprise conditions of culture medium pH, ionic strength, nutritive content, etc.; temperature; oxygen/CO₂/nitrogen content; humidity; light and other culture conditions that permit production of the compound by the host microorganism or plant, i.e., by the metabolic action of the microorganism or plant. Appropriate culture conditions are well known for microorganisms and plants (including plant cells) that can serve as host cells.

It is understood that a range of microorganisms and plants can be modified to include a recombinant metabolic pathway suitable for the production of other chemicals such as n-butanol, n-hexanol and octanol. It is also understood that various microorganisms or plants can act as “sources” for genetic material encoding target enzymes suitable for use in a recombinant microorganism or plant provided herein.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the procaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt ([NaCl]); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; and (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The disclosure includes recombinant microorganisms that comprise at least one recombinant enzymes of the rGS pathway set forth in FIGS. 1, 2 and 5. For example, chemoautotrophs, photoautotroph, and cyanobacteria can comprise native malate thiokinase enzymes, accordingly, overexpressing sucC-2/sucD-2 by tying expression to a non-native promoter can produce metabolite to drive the rGS pathway when combined with the other appropriate enzymes of FIG. 1, 2 an 5. Additional enzymes can be recombinantly engineered to further optimize the metabolic flux, including, for example, balancing ATP, NADH, NADPH and other cofactor utilization and production.

In another embodiment, a method of producing a recombinant microorganism that comprises optimized carbon utilization including a rGS pathway to convert 4 carbon substrates such as succinate to acetyl-CoA or other metabolites derived therefrom including, but not limited to, 1-butanol, 2-pentanone, isobutanol, n-hexanol and/or octanol is provided. The method includes transforming a microorganism with one or more recombinant polynucleotides encoding polypeptides selected from the group consisting of a malate thiokinase (e.g., sucC-2/sucD-2), a malyl-CoA lyase (e.g., mcl1), and an isocitrate lyase (e.g., aceA).

In another embodiment, as mentioned previously, a recombinant organism as set forth in any of the embodiments above, is cultured under conditions to express any/all of the enzymatic polypeptide and the culture is then lysed or a cell free preparation is prepared having the necessary enzymatic activity to carry out the pathway set forth in FIG. 1, 2 or 5 and/or the production of a 1-butanol, isobutanol, n-hexanol, octanol, 2-pentanone among other products (see, e.g., FIGS. 12A-F).

In addition to microorganisms, the pathways of the disclosure can be engineered into plants to obtain transgenic or recombinant plants that produce acetyl-CoA from a 4-carbon substrate.

Carbon fixation is the process by which carbon dioxide is incorporated into organic compounds. In the process of transforming sunlight into biological fuel, plants absorb carbon dioxide and water. Carbon fixation in plants and algae is achieved by the Calvin-Benson Cycle. The productivity of the Calvin-Benson cycle is limited, under many conditions, by the slow rate and lack of substrate specificity of the carboxylating enzyme Rubisco. Several lines of evidence indicate that in-spite of its shortcomings, Rubisco might already be naturally optimized and hence its potential for improvement is very limited. The disclosure provides an alternative pathways that can support carbon fixation with a higher rate in the efforts towards sustainability.

According to one embodiment of the disclosure, the polynucleotides of the disclosure are expressed in cells of a photosynthetic organism (e.g. higher plant, algae or cyanobacteria). The term ‘“plant” as used herein encompasses whole plants, ancestors and progeny of the plants and plant parts, including seeds, shoots, stems, roots (including tubers), and plant cells, tissues and organs. The plant may be in any form including suspension cultures, embryos, meristematic regions, callus tissue, leaves, gametophytes, sporophytes, pollen, and microspores. Plants that are particularly useful in the methods of the disclosure include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Rpbinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of the disclosure.

Expression of polynucleotides encoding enzymes of the rGS pathway of the disclosure can be from tissue specific, inducible or constitutive promoters. Examples of constitutive plant promoters include, but are not limited to CaMV35S and CaMV19S promoters, tobacco mosaic virus (TMV), FMV34S promoter, sugarcane bacilliform badnavirus promoter, CsVMV promoter, Arabidpsis ACT2/ACT8 actin promoter, Arabidpsis ubiquitin UBQ 1 promoter, barley leaf thionin BTH6 promoter, and rice actin promoter.

An inducible promoter is a promoter induced by a specific stimulus such as stress conditions comprising, for example, light, temperature, chemicals, drought, high salinity, osmotic shock, oxidant conditions or in case of pathogenicity. Examples of inducible promoters include, but are not limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr203J and str246C active in pathogenic stress.

Nucleic acid constructs comprising one or more enzymes of the rGS pathway can be introduced into plant cells using Ti plasmid, Ri plasmid, plant viral vectors, direct DNA transformation, microinjection, electroporation, Biolistics (gene gun) and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the disclosure.

It will be appreciated that other than containing the necessary elements for the transcription and translation of the inserted coding sequence (encoding the polypeptide), the expression construct of the disclosure can also include sequences engineered to optimize stability, production, purification, yield or activity of the expressed polypeptide.

The enzymes of the disclosure can be expressed with chloroplast targeting peptides. Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Various methods can be used to introduce the expression vector of the disclosure into the host cell system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et al., [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Plant cells may be transformed stably or transiently with the nucleic acid constructs of the disclosure. In stable transformation, the nucleic acid molecule of the disclosure is integrated into the plant genome and as such it represents a stable and inherited trait. In transient transformation, the nucleic acid molecule is expressed by the transformed cell, but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches: (i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112; and (ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is preferred, transient transformation of leaf cells, meristematic cells or the whole plant is also envisaged by the disclosure.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well, as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the disclosure is demonstrated by the above references as well as in U.S. Pat. No. 5,316,931.

In addition to the above, the nucleic acid molecule of the disclosure can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a one or more polynucleotides encoding rGS enzymes, at least one nucleic acid stretch which is derived from the chloroplast's genome. In addition, the exogenous nucleic acid can include a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

It will be appreciated that any of the construct types used in the disclosure can be co-transformed into the same organism (e.g. plant) using same or different selection markers in each construct type (e.g., one or more constructs can be used, each with one or more enzymes of an rGS pathway). Alternatively a first construct type can be introduced into a first plant while a second construct type can be introduced into a second isogenic plant, following which the transgenic plants resultant therefrom can be crossed and the progeny selected for double transformants. Further self-crosses of such progeny can be employed to generate lines homozygous for both constructs.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”), each of which is incorporated herein by reference in its entirety.

Examples of protocols sufficient to direct persons of skill through in vitro amplification methods, including the polymerase chain reaction (PCR), the ligase chain reaction (LCR), Qβ-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13:563-564.

Improved methods for cloning in vitro amplified nucleic acids are described in Wallace et al., U.S. Pat. No. 5,426,039.

Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

The disclosure thus provides a plant exhibiting artificially introduced rGS pathways genes, wherein the plant exhibits improved photosynthesis. The disclosure also provides methods of improving the plant biomass and making a commodity product comprising: (a) obtaining a plant exhibiting expression or overexpression of various rGS genes, wherein the sugar content of the plant is increased when compared to a plant that lacks the rGS pathway expression; or (b) obtaining a plant exhibiting expression or overexpression of various rGS genes, wherein the oil content of the plant is increased when compared to a plant that lacks the rGS pathway expression.

The disclosure further provides novel methods and compositions for improving a photosynthetic pathway. In addition, the disclosure provides transgenic/recombinant plants comprising a non-native photosynthetic pathway that can be adapted by the plants and can perform better than the existing rubisco dependent pathway. The disclosure demonstrates for the first time that artificially introduced CO₂ fixing system can complement sbpase mutant. The sbpase is an important enzyme to complete the Calvin cycle and in Arabidopsis, there is no other isoform is reported in plants. The studies described herein demonstrate that an alternate system can provide an energy efficient system to fix CO₂ in the plants and also effectively produce the higher biomass compared to the photosynthetic system operated by Rubisco.

The invention is illustrated in the following examples, which are provided by way of illustration and are not intended to be limiting.

### EXAMPLES

### Strain Construction

All strains used in that study are listed in Table 1. JCL16 (rrnB_{T14} ΔlacZ_{WJ16} hsdR514 ΔaraBAD_{AH33} ΔrhaBAD_{LD78}/F′ [traD36 proAB+ lacI^{q}ZΔM15]) was used as the wild type (WT) (Atsumi et al., 2008). XL-1 Blue (Stratagene) was used to propagate all plasmids. BL-21 DE3 (Invitrogen) was used to express enzymes prior to enzyme assays. Gene deletions were carried out by P1 transduction using single knockout strains from the Keio collection (Baba et al., 2006). Each knockout was verified by PCR using the following primers flanking the deleted locus:

gltA
(5′-GTTGATGTGCGAAGGCAAATTTAAG-3′ (SEQ ID NO: 11) +
5′-AGGCATATAAAAATCAACCCGCCAT-3′(SEQ ID NO: 12)),
prpC
(5′-GTATTCGACAGCCGATGCCTGATG-3′ (SEQ ID NO: 13) +
5′-CTTTGATCATTGCGGTCAGCACCT-3′ (SEQ ID NO: 14)),
mdh
(5′-TTCTTGCTTAGCCGAGCTTC-3′ (SEQ ID NO: 15) +
5′-GGGCATTAATACGCTGTCGT (SEQ ID NO: 16),
mqo
(5′-GACTGCTGCCGTCAGGTCAATATG-3′ (SEQ ID NO: 17) +
5′-CTCCACCCCGTAGGTTGGATAAGG-3′ (SEQ ID NO: 18)),
ppc
(5′-ACCTTTGGTGTTACTTGGGGCG-3′ (SEQ ID NO: 19) +
5′-TACCGGGATCAACCACAGCGAA-3′ (SEQ ID NO: 20)),
aceB
(5′-CTATTTCCCGCACAATGATCCGCA-3′ (SEQ ID NO: 21) +
5′-CTTCAATACCCGCTTTCGCCTGTT-3′ (SEQ ID NO: 22)),
citE
(5′-GCGACTGAAACGCTATGCCGAA-3′ (SEQ ID NO: 23) +
5′-TTCAGTTCGCCGCTCTGTACCA-3′ (SEQ ID NO: 24)),
icd
(5′-GTTTACCCGGCTGGGTTAA-3′ (SEQ ID NO: 25) +
5′-AGTCACGATCGTTAGCAATTG-3′ (SEQ ID NO: 26)).

TABLE 1
Strains and plasmids used in the study.
STRAINS

| # in | Strain | | | |
|---|---|---|---|---|
| text | name | Relevant genotype | Plasmid(s) | Reference |
| | JCL16 | rrnBT14 ΔlacZWJ16 hsdR514 | — | Atsumi et |
| | | ΔaraBAD_{AH33} ΔrhaBAD_{LD78}/F'[traD36 | | al., 2008 |
| | | proAB⁺ lacI^{q}Z ΔM15] | | |
| | JW3928 | BW25113 (rrnB3 ΔlacZ4787 hsdR514 | — | Baba et al., |
| | | Δ(araBAD)567 Δ(rhaBAD)568 rph-1 | | 2006 |
| | | Δppc | | |
| | SM43 | JW3928 | pSM13 | This work |
| | SM44 | JW3928 | pSM22 | This work |
| 1 | SM160 | JCL16 ΔgltA ΔprpC | pSM22 pSMc00 pYK | This work |
| 2 | SM161 | JCL16 ΔgltA ΔprpC | pSM22 pSMc00 pLG5 | This work |
| 3 | SM163 | JCL16 ΔgltA ΔprpC | pSM22 pSM12 pLG5 | This work |
| 4 | SM162 | JCL16 ΔgltA ΔprpC | pSM22 pSM11 pLG5 | This work |
| 5 | SM164 | JCL16 ΔgltA ΔprpC | pSM22 pSM62 pYK | This work |
| 6 | SM165 | JCL16 ΔgltA ΔprpC | pSM22 pSM62 pLG5 | This work |
| 7 | SM167 | JCL16 ΔgltA ΔprpC | pSM22 pSM62ΔMTK pLG5 | This work |
| 8 | SM166 | JCL16 ΔgltA ΔprpC | pSM22 pSM62ΔMCL pLG5 | This work |
| 9 | SM169 | JCL16 ΔgltA Δmdh Δppc ΔcitE Δmqo | pSM01 pYK | This work |
| 10 | SM170 | JCL16 ΔgltA Δmdh Δppc ΔcitE Δmqo | pSM01 pGltA | This work |
| 11 | SM172 | JCL16 ΔgltA Δmdh Δppc Δmqo | pSM01 pYK | This work |
| 12 | SM171 | JCL16 ΔgltA Δmdh Δppc ΔcitE Δmqo | pSM01 pSMb02 | This work |
| 13 | SM93a | JCL16 ΔgltA Δmdh Δppc ΔcitE Δmqo | pSMf02 pLG5 pSM69 | This work |
| | | ΔaceB | | |
| 14 | SM93b | JCL16 ΔgltA Δmdh Δppc ΔcitE Δmqo | pSMf02 pLG5 pSM70 | This work |
| | | ΔaceB | | |
| 15 | SM93c | JCL16 ΔgltA Δmdh Δppc ΔcitE Δmqo | pSMf02 pLG5 pSM71 | This work |
| | | ΔaceB | | |
| 16 | SM135a | JCL16 ΔgltA Δmdh Δppc ΔcitE Δmqo | pSMf02 pLG5 pSM69 | This work |
| | | ΔaceB Δicd | | |
| 17 | SM135b | JCL16 ΔgltA Δmdh Δppc ΔcitE Δmqo | pSMf02 pLG5 pSM70 | This work |
| | | ΔaceB Δicd | | |
| 18 | SM135c | JCL16 ΔgltA Δmdh Δppc ΔcitE Δmqo | pSMf02 pLG5 pSM71 | This work |
| | | ΔaceB Δicd | | |
| 19 | SM178 | JCL16 ΔgltA Δmdh Δppc ΔcitE Δmqo | pSM22★ pSM73★ | This work |
| | | ΔaceB Δicd | pSMf02★ pSM62+★ | |
| 20 | SM179 | JCL16 ΔgltA Δmdh Δppc ΔcitE Δmqo | pSM22 pSM73 pSMf00 | This work |
| | | ΔaceB Δicd | pSM62+ | |
| 21 | SM181 | JCL16 ΔgltA Δmdh Δppc ΔcitE Δmqo | pSM22 pSM73 pSMf02 | This work |
| | | ΔaceB Δicd | pSM62+ ΔMCL | |
| 22 | SM180 | JCL16 ΔgltA Δmdh Δppc ΔcitE Δmqo | pSM22 pYK pSMb02 | This work |
| | | ΔaceB Δicd | pSM62+ | |

PLASMIDS

| Plamid name | Description | Reference |
|---|---|---|
| pSS25 | CDF-ori, SpR, LacI, PLlacO1:his-tag aceA(Ec) | This work |
| PXL18-4 | ColE1-ori, SpR, lacI, PLlacO1:AclB(Ct):RBS:AclA(Ct) his-tag | This work |
| pSMg45 | CDF-ori, SpR, LacI, T7:his-tag SucCD-2 (Mc) | This work |
| pSMg59 | CDF-ori, SpR, LacI, T7:his-tag Mcl1 (Rs) | This work |
| pGltA | ColA ori, Km^{R}, LacI, P_{L}lacO1:GltA(Ec) | This work |
| pLG5 | ColA ori, Km^{R}, LacI, P_{L}lacO1:AceA(Ec) | This work |
| p5M22★ | pSC101* ori, Sp^{R}, P_{L}lacO1:DctA(Bs) | This work |
| pSM69 | pSC101* pro, Sp^{R}, P_{L}lacO1:AcnA(Ec) | This work |
| pSMf02★ | p15A ori, Amp^{R}, LacI, P_{L}lacO1:AclB(Ct):RBS:AclA(Ct) | This work |
| pSMb02 | ColA ori, Km^{R}, LacI, P_{L}lacO1:AclB(Ct):RBS:AclA(Ct) | This work |
| pSM70 | pSC101* ori, Sp^{R}, P_{L}lacO1:AcnB(Ec) | This work |
| pSM71 | pSC101* ori, Sp^{R}, empty | This work |
| pYK | ColA ori, Km^{R}, LacI, empty | This work |
| pSMc00 | p15A ori, Cm^{R}, empty | This work |
| pSM11 | p15A ori, Cm^{R}, P_{L}lacO1:GlcB(Ec) | This work |
| pSM12 | p15A ori, Cm^{R}, P_{L}lacO1:AceB(Ec) | This work |
| pSM73★ | ColA ori, Km^{R}, LacI, P_{L}lacO1:AceA(Ec), P_{L}lacO1:AcnA(Ec) | This work |
| pSM13 | pSC101* ori, Sp^{R}, P_{L}lacO1:DctA(Ec) | This work |
| pSM62 | p15A ori, Cm^{R}, P_{L}lacO1:SucCD-2(Mc):RBS:Mcl1(Rs) | This work |
| pSM62ΔMCL | p15A ori, Cm^{R}, P_{L}lacO1:SucCD-2(Mc) | This work |
| pSM62ΔMTK | p15A ori, Cm^{R}, P_{L}lacO1:Mcl(RS) | This work |
| pSM62+★ | ColE1 ori, Cm^{R}, P_{L}lacO1:SucCD-2(Mc):RBS:Mcl1(Rs) | This work |
| pSM62+ΔMCL | ColE1 ori, Cm^{R}, P_{L}lacO1:SucCD-2(Mc) | This work |
| pSM01 | pSC101* ori, Amp^{R}, P_{L}lacO1:CitA(Se) | This work |

SpR: Spectinomycin resistant;
KmR: Kanamycin resistant;
AmpR: Ampicillin resistant;
CmR: Chloramphenicol resistant;
RBS: 5′---AGGAGA---3′;
Bs: Bacillus subtilis;
Ec: Escherischia coli;
Ct: Chlorobium tepidum;
Mc: Methylococcus capsulatus;
Rs: Rhodobacter sphaeroides;
Se: Salmonella enterica.
★Plasmids used in final, full---pathway strain.

Plasmid Construction.

All plasmids used in this study were assembled using isothermal DNA assembly, as described by Gibson et al. (2009). Briefly, backbone of the plasmid and insert(s), overlapping by 16-20 bp on each end, were PCR-amplified using iProof polymerase (Biorad). DNA amplicons of the expected size were gel-purified and mixed in equimolar amounts in a final volume of 5 μL. 15 μL of a reaction mix [6.65% PEG-8000, 133 mM Tris-HCl, pH 7.5, 13.3 mM MgCl₂, 13.3 mM DTT, 0.27 mM each of the four dNTPs, 1.33 mM NAD⁺, 0.08 U T5 exonuclease (Epicentre), 0.5 U Phusion Polymerase (NEB), 80 U Taq DNA ligase (NEB) in water] was added, thoroughly pipet-mixed with the DNA, and incubated at 50° C. for 1 hour. 5 μL of the assembly mixture were transformed in Z-competent (Zymo Research) XL1-blue E. coli cells (Agilent) according to manufacturer's recommendations, and plated on LB Agar plates containing the appropriate antibiotic. At least 3 independent resulting colonies were cultured, their plasmid purified, and verified by sequencing.

All plasmid used in that study and their features are listed in Table 1.

Growth Conditions.

For general molecular biology purposes Escherichia coli strains were grown in Luria Bertani (LB) medium at 37° C. and agitation rates of 200 rpm. For strains containing plasmids the medium was supplemented with the appropriate antibiotic at the following concentrations: Kanamycin 50 μg/mL, Chloramphenicol 30 μg/mL, Ampicillin 50-100 μg/mL, Spectinomycin 100 μg/mL (all antibiotics were purchased from Sigma Aldrich).

For selections on minimal medium cells were first grown to mid-log phase in LB medium and induced with 0.1 mM Isopropyl-β-D-thio-galactoside (IPTG, Gold Biotechnology) for three hours to ensure expression of the proteins of interest. Cells from 1 mL of medium were then harvested by centrifugation at 5000×g and washed once with equal volumes of minimal medium. The cells were resuspended in 1 mL of minimal medium and streaked out on selective plates. The selective plates contained M9 minimal medium, 2% glucose, 1 mM MgSO₄, 0.1 mM CaCl₂, 0.1 mg/mL thiamine hydrochloride, 0.1 mM IPTG and the appropriate antibiotics. As noted in the text the plates were supplemented with a combination of 10 mM aspartate, 10 mM glutamate, 10 mM citrate, 10 mM glyoxylate, 10 mM succinate or 10 mM malate (all sodium salts from Sigma Aldrich).

Enzyme Assays. Isocitrate Lyase (ICL) Enzyme Purification and Assay:

His-tagged E. coli AceA was over-expressed from plasmid pSS25 in E. coli BL21(DE3) cells by inoculating LB medium supplemented with spectinomycin 25 mg/L with a 1/100 dilution of an overnight culture. Cells were grown at 37° C. with agitation rates of 200 rpm to mid-log phase and induced with 0.1 mM IPTG. The culture was grown for an additional 3 hours under the same conditions and cells were then harvested by centrifugation. Cells were lysed in His-binding buffer (Zymo Research) by using the bead beater method (TissueLyser II from Qiagen), and were then centrifuged to pellet cell debris. Supernatant was applied to a His-Spin Protein Miniprep column (Zymo Research) and purified according to manufacturers instructions. Concentration of purified protein elute was determined using the BioRad Protein Assay kit, and protein purity was verified by standard SDS-PAGE and Coomassie staining methods. Purified protein was kept on ice and used the same day.

To assay the activity of ICL, the production of isocitrate was coupled to the activity of isocitrate dehydrogenase (ICD), which oxidizes and decarboxylates isocitrate to α-ketoglutarate, while reducing NADP⁺ to NADPH. The production of NADPH can be followed spectrophotometrically. Reactions were performed at room temperature in UV cuvettes and monitored at 340 nm. The reaction mixture contained 50 mM Tris-HCl, pH 7.5, 100 mM NaCl, 5 mM MgCl₂, 1 mM dithiothreitol, 5 mM NADP⁺, 0.1× commercial Bacillus subtilis ICD (Sigma Aldrich), and, if appropriate, 10 mM sodium succinate (Sigma Aldrich) and 10 mM sodium glyoxylate (Sigma Aldrich) and 18.75 μg/mL of purified protein.

Coupled Malate Thiokinase (MTK) and Malyl-CoA Lyase (MCL) Enzyme Assay.

Putative native MTK operons placed under the control of the T7 promoter (See supplementary methods) were expressed in E. coli BL21(DE3) cells by inoculating LB medium supplemented with spectinomycin 25 mg/L with a 1/100 dilution of an overnight culture. Cells were grown at 37° C. with agitation rates of 200 rpm to mid-log phase and induced with 0.1 mM IPTG. The culture was grown for an additional 5 hours at 25° C. and cells were then harvested by centrifugation. Cells were lysed in 0.1 M Tris-Cl pH 7.5 by using the bead beater method (TissueLyser II from Qiagen) and were then centrifuged to pellet cell debris. Concentration of the total soluble protein extract was determined using the BioRad Protein Assay kit. Total soluble extracts were kept on ice and used the same day.

MTK activity was tested in a coupled enzyme assay with purified His-tagged MCL (see below). MTK performs the ATP-dependent condensation of malate and CoA into malyl-CoA. In turn, MCL cleaves malyl-CoA into acetyl-CoA and glyoxylate, the latter reacting with phenylhydrazine to form glyoxylate-phenylhydrazone. Formation of glyoxylate-phenylhydrazone is recorded at 324 nm. Reactions were set up at 37° C. in a final volume of 100 μL containing 50 mM Tris-Cl pH 7.5, 5 mM MgCl₂, 2 mM phenylhydrazine, 10 mM malate, 2 mM ATP, 0.85 μg purified MCL (see below), and 0.2-2 lag soluble protein extract. Reactions were started by the addition of CoA to a final concentration of 1 mM, except for C. auriantacus SmtAB where succinyl-CoA 1 mM was used. Similar to malate thiokinase, succinyl-CoA:l-malate CoA transferase (SmtAB) produces malyl-CoA from malate, but uses succinyl-CoA as the Co-A donor instead of free Co-A. Specific enzyme activities were calculated based on a glyoxylate standard curve (0-10-20-30-40 nmoles glyoxylate in 100 μL reaction buffer).

Malyl-CoA Lyase (MCL) Enzyme Purification.

His-tagged R. sphaeroides MCL was over-expressed from plasmid pSMg59 in E. coli BL21(DE3) cells by inoculating LB medium supplemented with spectinomycin 25 mg/L with a 1/100 dilution of an overnight culture. Cells were grown at 37° C. with agitation rates of 200 rpm to mid-log phase and induced with 0.1 mM IPTG. The culture was grown for an additional 3 hours under the same conditions and cells were then harvested by centrifugation. Cells were lysed in His-binding buffer (Zymo Research) by using the bead beater method (TissueLyser II from Qiagen) and were then centrifuged to pellet cell debris. Supernatant was applied to a His-Spin Protein Miniprep column (Zymo Research) and purified according to manufacturers instructions. Concentration of purified protein elute was determined using the BioRad Protein Assay kit, and protein purity was verified by standard SDS-PAGE and Coomassie staining methods. Purified protein was kept on ice and used the same day.

ATP-Citrate Lyase (ACL) Enzyme Purification and Assay.

His-tagged C. tepidum AclBA was over-expressed from plasmid pXL18-4 in E. coli BL21(DE3) cells by inoculating LB medium supplemented with spectinomycin 50 mg/L with a 1/100 dilution of an overnight culture. Cells were grown at 37° C. with agitation rates of 200 rpm to mid-log phase and induced with 0.1 mM IPTG. The culture was grown for an additional 20 hours at room temperature with agitation rates of 200 rmp and cells were then harvested by centrifugation. Cells were lysed in His-binding buffer (Zymo Research) by using the bead beater method (TissueLyser II from Qiagen) and were then centrifuged to pellet cell debris. Supernatant was applied to a His-Spin Protein Miniprep column (Zymo Research) and purified according to manufacturers instructions. Concentration of purified protein elute was determined using the BioRad Protein Assay kit, and protein purity was verified by SDS-PAGE. Purified protein was kept frozen at −80° C. in 20% glycerol and used the next day.

To assay the activity of ACL, the production of oxaloacetate was coupled to the activity of malate dehydrogenase (MDH), which reduces oxaloacetate to malate, while oxidizing NADH to NAD⁺. The consumption of NADH can be followed spectrophotometrically. Reactions were performed at room temperature in UV cuvettes and monitored at 340 nm. The reaction mixture contained 100 mM Tris-HCl, pH 8.4, 10 mM MgCl₂, 10 mM dithiothreitol, 0.25 mM NADH, 3.3 U/mL commercial porcine heart MDH (Sigma Aldrich), and, if appropriate, 20 mM sodium citrate (Sigma Aldrich), 0.44 mM coenzyme A (Sigma Aldrich), 2.5 mM Adenosine triphosphate (ATP) and 1.283 μg/mL of purified protein.

Reversibility of Isocitrate Lyase.

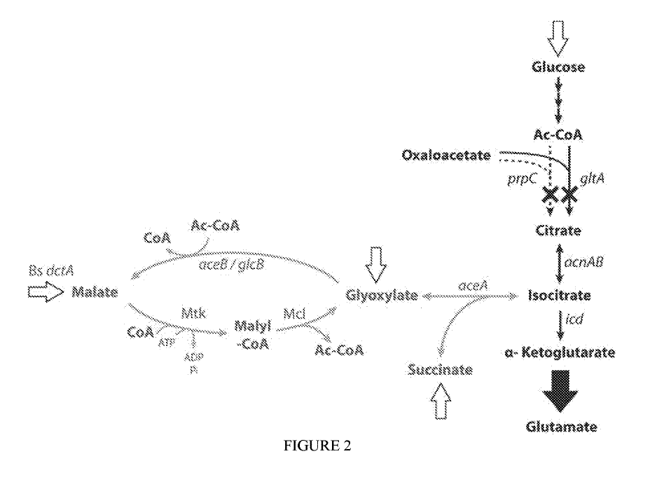

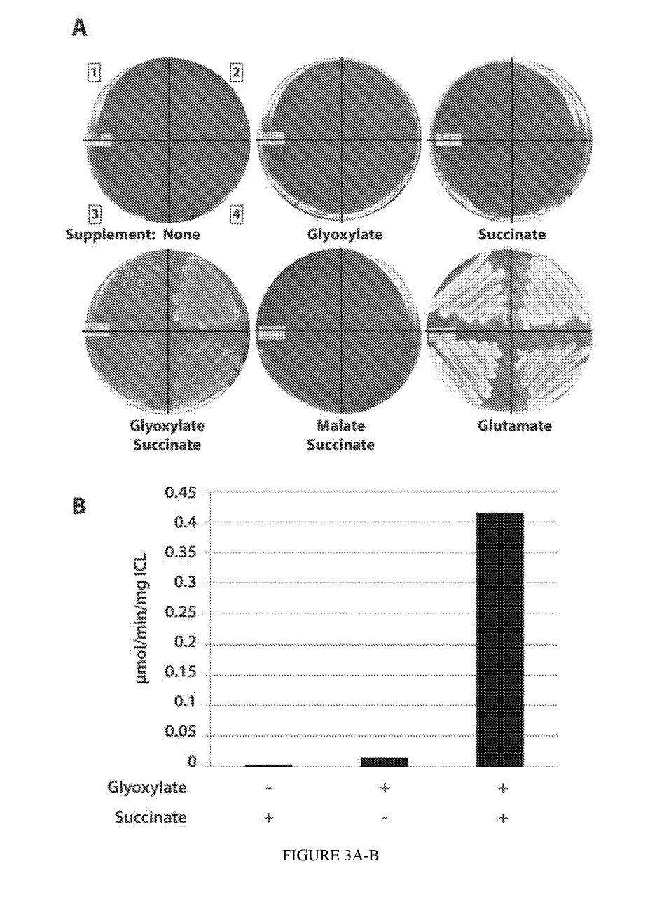

A genetic selection system was developed to test for reversibility of the glyoxylate shunt enzymes in vivo (FIG. 2). The first enzyme of the glyoxylate shunt, ICL is encoded by the E. coli gene aceA. The reversibility of ICL was tested based on its ability to convert succinate and glyoxylate to isocitrate, which is a precursor for glutamate synthesis. Normally, glutamate is synthesized through intermediates of the TCA cycle. By deleting citrate synthase (coded by gltA), E. coli becomes a glutamate auxotroph. To avoid a second-site mutation that complements ΔgltA, we also deleted prpC, which codes for a proprionate inducible methylcitrate synthase that has minor citrate synthase activity (Maloy and Nunn, 1982), resulting glutamate auxotroph selection strain (ΔgltA ΔprpC) is hereafter referred to as the Glu⁻ strain (FIG. 2 and table 1). In the glyoxylate shunt, ICL cleaves isocitrate into glyoxylate and succinate. Therefore, if ICL is active in the reverse, isocitrate-forming, direction, the Glu⁻ strain expressing ICL is expected to grow on glucose minimal media supplemented with glyoxylate and succinate. As presented in FIG. 3A, the strain overexpressing Ec AceA using a strong, IPTG-inducible promoter (P_{L}lacO1) was able to grow in the absence of glutamate when both glyoxylate and succinate were supplied in the medium (Strain 2, FIG. 3A). This same strain was not able to grow when only glyoxylate or only succinate was added in the medium. A strain where AceA was not overexpressed served as a control (Strain 1, FIG. 3A). This strain was not able to grow on medium supplemented with both glyoxylate and succinate. These results suggest that AceA is reversible in vivo and able to form isocitrate from glyoxylate and succinate. The fact that wild-type expression levels of aceA from the chromosome did not allow for growth under these conditions, is most likely due to the repression of aceA under the growth condition (Cozzone, 1998), which lacks the inducer acetate and contains the repressor glucose. The reversibility of E. coli AceA was also confirmed in vitro (FIG. 3B). The enzyme was His-tagged and purified, and showed reverse (condensing) activity in an enzyme assay, where production of isocitrate was coupled with NADP⁺ reduction by commercial isocitrate dehydrogenase. Formation of NADPH was followed spectrophotometrically. Production of isocitrate was also confirmed HPLC analysis by comparison to known standards.

Irreversibility of Malate Synthase.

The enzyme MS acetylates glyoxylate to form malate in the glyoxylate shunt in its native direction. Reversal of this reaction is unfavorable (ΔᵣG′°=44.4 kJ/mol for glyoxylate formation) (Alberty, 2006). However, if reversed, MS would convert malate to acetyl-CoA and glyoxylate. We tested for this reverse activity in the Glu⁻ strain overexpressing aceA. In this strain, any glyoxylate produced from malate could act as a substrate for ICL to be condensed with succinate, forming isocitrate and rescuing growth. Unfortunately, malate is transported very poorly into E. coli when glucose is present in the growth medium (Davies et al., 1999).

To solve the malate transport problem, the efficiency of this transport step was examined by using a Δppc strain, which cannot grow in glucose minimal medium unless supplemented with a TCA cycle intermediate, such as malate. Consistent with the previous report (Ashworth and Kornberg, 1966), the Δppc strain JW3928 (Baba et al., 2006) cannot grow on minimal medium supplemented by glucose, and it grew poorly when a malate supplement was added (Table 2). Overexpression of the E. coli malate transporter dctA did not help malate uptake under these conditions (Strain SM43, Table 2). However, overexpression of the Bacillus subtilis dctA (Bs DctA) (Groeneveld et al., 2010) gene, which is not regulated by glucose in the same way as the E. coli enzyme is, did allow for fast growth of the Δppc mutant on M9 supplemented with glucose and malate (Strain SM44, Table 2).

TABLE 2
Bacillus subtilis DctA transporter allows malate uptake in E. coli
Δppc mutant. Growth of E. coli strains JW3928, SM43 and SM44
were grown on M9 plates 2% Glucose 100 μM IPTG with no
supplements, or supplemented with 20 mM malate or succinate.

| | | Gene | Growth | Growth on | Growth on |
|---|---|---|---|---|---|
| Strain | Relevant | over- | on M9 | M9 glucose + | M9 glucose + |
| name | mutation | expressed | glucose | malate | succinate |
| JW3928 | Δppc | none | − | + | +++ |
| SM43 | Δppc | Ec DctA | − | + | +++ |
| SM44 | Δppc | Bs DctA | − | +++ | +++ |

−−−: no growth; +: poor growth; +++: healthy growth.
Plate photographs are shown in supplementary FIG. 1.

With the malate transport problem solved, the reversibility of MS was tested by using the Glu⁻ strain overexpressing malate transporter (Bs DctA) and E. coli MS. Two isoenzymes of MS exist in E. coli, and they are coded by aceB and glcB. No growth on selective plates (malate and succinate supplements in glucose minimal medium) was observed when E. coli aceB or glcB were overexpressed together with Bs dctA and Ec aceA (Strains 3 and 4, FIG. 3A), indicating that, as expected, the E. coli MS enzymes are not active enough in the reverse direction to support growth in the selection. Interestingly, the growth of strains overexpressing the MS genes in addition to ICL actually appeared to be retarded on plates supplemented with glyoxylate and succinate. This could be further evidence of the irreversibility of MS, as this growth retardation could be due to glyoxylate being drained away from ICL by the MS acting in the forward direction.

Converting Malate to Glyoxylate and Acetyl CoA.

To find a suitable alternative to E. coli MS, to metabolize malate into glyoxylate and acetyl-CoA, enzymes were sought that would couple this reaction with the hydrolysis of ATP to drive it in the desired direction. Such enzymes can be found in the serine cycle of type II methylotrophs, such as Methylobacterium extorquens. Here malyl-CoA is formed from malate and CoA by an ATP-dependent malate thiokinase (MTK; ΔᵣG′°=−7.7 kJ/mol)(Ablerty, 2006). Malyl-CoA is then cleaved into glyoxylate and acetyl-CoA by a malyl-CoA lyase (MCL; ΔᵣG′°=14.5 kJ/mol) (Alberty, 2006; Hanson and Hanson, 1996). MCLs are also involved in the 3-hydroxypropionate CO₂ fixation pathway found in Chloroflexus auriantacus, and (in the condensing direction) in the ethylmalonyl-CoA pathway of Rhodobacter sphaeroides and others. The activity of MTK/MCL combinations were tested in vivo by employing the same selection used to evaluate AceB and GlcB reversibility. The enzymes were expressed together with Bs DctA, Ec AceA in the Glu⁻ strain, and tested for growth on medium containing malate and succinate. Initially the well-characterized genes M. extorquens MtkAB and MclA (Chistoserdova and Lidstrom, 1994) (Chistoserdova and Lindstrom, 1997) were tested, and found that expression of these genes together did not rescue growth of the Glu⁻ selection strain, possibly due to expression problems in E. coli.

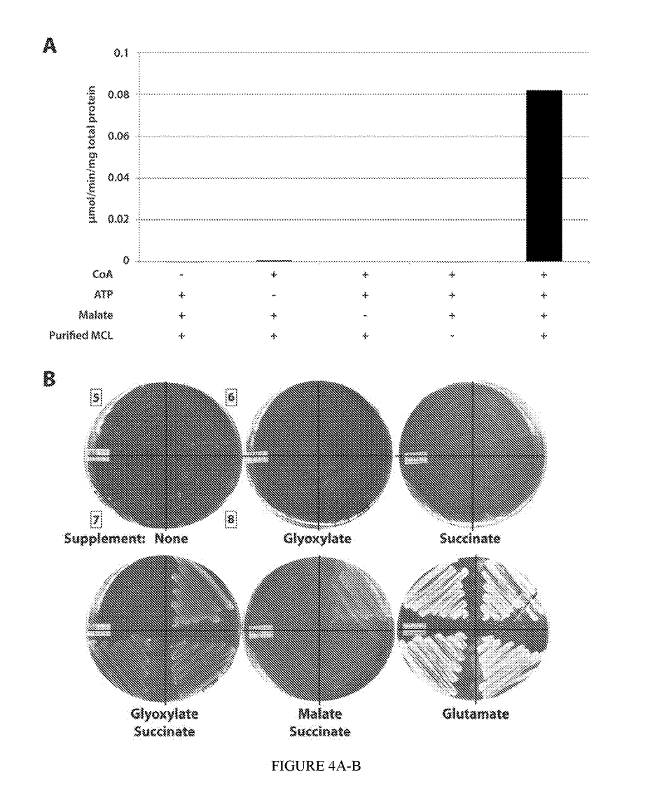

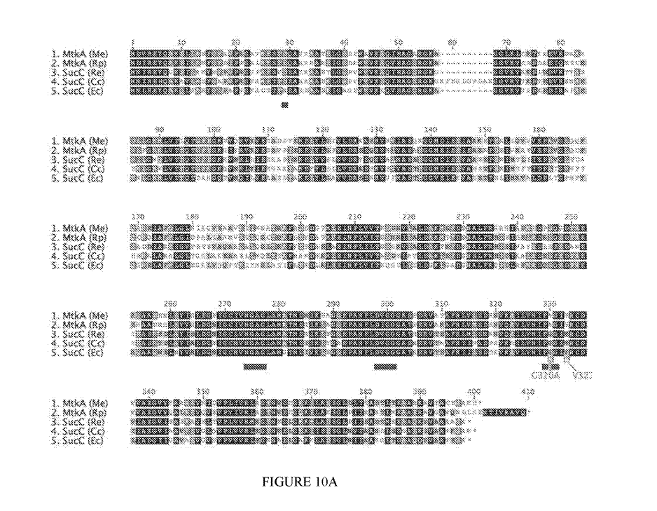

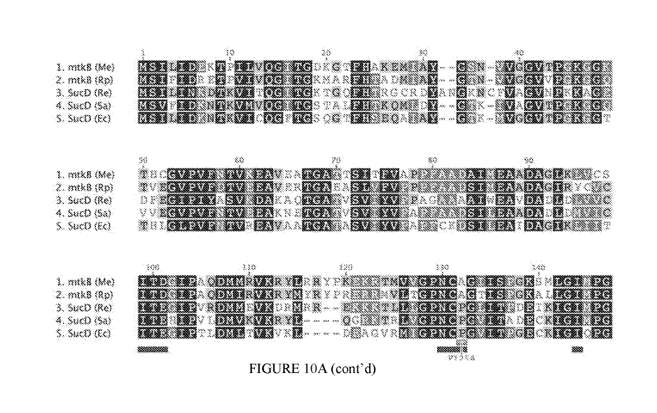

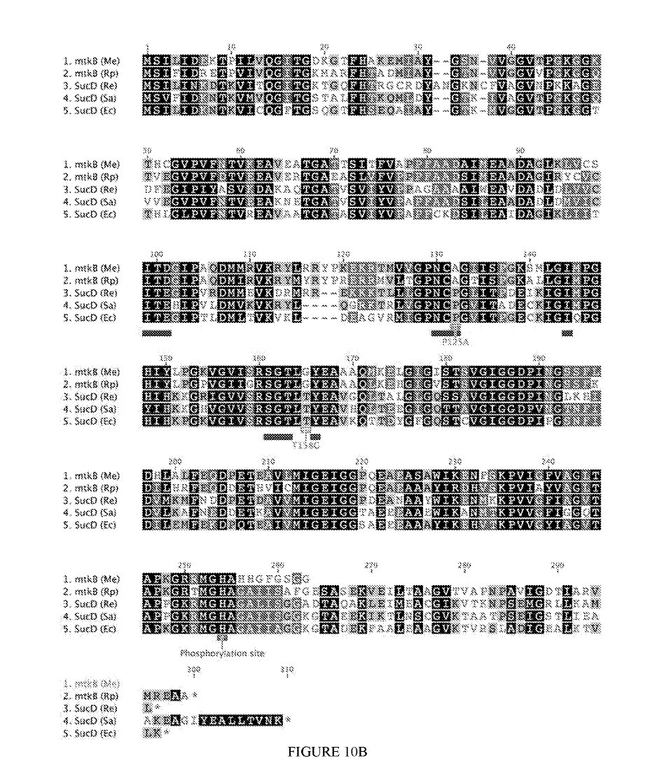

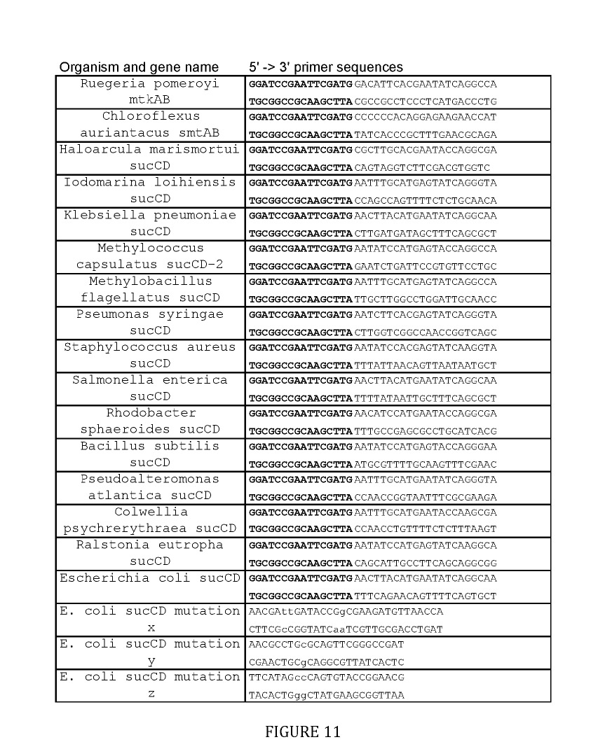

Therefore, homologous enzymes from various organisms were expressed in E. coli and tested in vitro for “reverse MS” activity to find the most active variant. Since Mcl1 from R. sphaeroides (Rs Mcl1) has been actively expressed in E. coli (Erb et al., 2010), this protein was purified and used it in excess in a coupled assay to test the activity of 15 putative MtkAB operons from various organisms expressed in E. coli (FIG. 9). In this screen, SucCD-2 from Methylococcus capsulatus (Ward et al., 2004) (Mc SucCD-2), expressed from plasmid pSMg45, showed the greatest MTK activity (FIG. 4A). Note that Mc SucCD-2 has been annotated as a succinyl-CoA synthetase, but, as shown here, has MTK activity. This enzyme was then tested in vivo (FIG. 4B). When expressed together in the Glu⁻ selection strain, Bs dctA, Mc sucCD-2, Rs=mcl1, and Ec AceA allowed for growth on glucose minimal medium with malate and succinate supplements, indicating that this MTK/MCL combination is active as a reverse MS (strain 6, FIG. 4B). Growth was observed (although more slowly) with addition of only succinate, which can be converted to malate by succinate dehydrogenase and fumarase. When ICL, MTK, or MCL was omitted (Strains 5, 7 or 8 respectively, FIG. 4B), no growth was observed on the selective plates, indicating that the overexpression of each enzyme is essential to the pathway in vivo.

These results show that malate can be converted to glyoxylate and acetyl-CoA at the expense of ATP. Therefore, by expressing Mc SucCD-2, Rs Mcl1, and Ec AceA, the glyoxylate shunt in E. coli is reversed, converting malate and succinate to acetyl-CoA and isocitrate using ATP to overcome the thermodynamic barrier.

Converting Citrate to Oxaloacetate and Acetyl-CoA.

With the input of two C₄ compounds malate and succinate, the output of the reversed glyoxylate shunt is one acetyl-CoA and the C₆ compound isocitrate. Therefore, the rGS was extend to convert isocitrate back to the C₄ compound OAA while releasing a second molecule of acetyl-CoA. This involved reversing two enzymatic steps that are shared with the TCA cycle: readily reversible aconitase (Gruer and Guest, 1994), as well as citrate synthase (CS), which is not expected to be reversible (ΔᵣG′°=40.3 kJ/mol for reverse reaction) (Alberty, 2006). In E. coli, the reverse CS reaction could be performed by the concerted action of the native enzymes citrate lyase (CL) (citrate→oxaloacetate+acetate; ΔᵣG′°=0.6 kJ/mol)(Alberty, 2006) and acetate:CoA ligase (acetate+CoA+ATP→acetyl-CoA+AMP+PPi; ΔᵣG′°=2.0 kJ/mol) (Alberty, 2006). An alternative is the non-native ATP-citrate lyase (ACL) that performs the ATP-dependent conversion of citrate directly to oxaloacetate and acetyl-CoA (ΔᵣG′°=2.7 kJ/mol) (Alberty, 2006). This enzyme is found in most eukaryotes, and archaea that fix carbon via the reductive TCA cycle (Fatland et al., 2002; Houston and Nimmo, 1984; and Hugler et al., 2007).

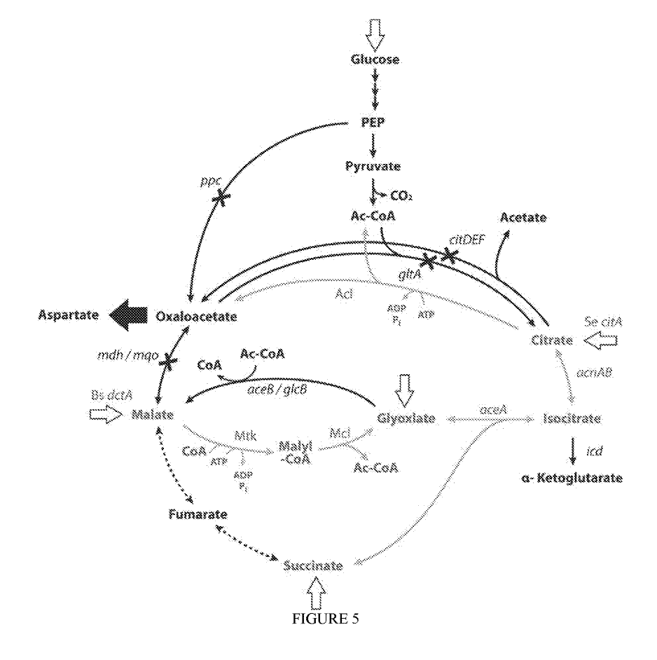

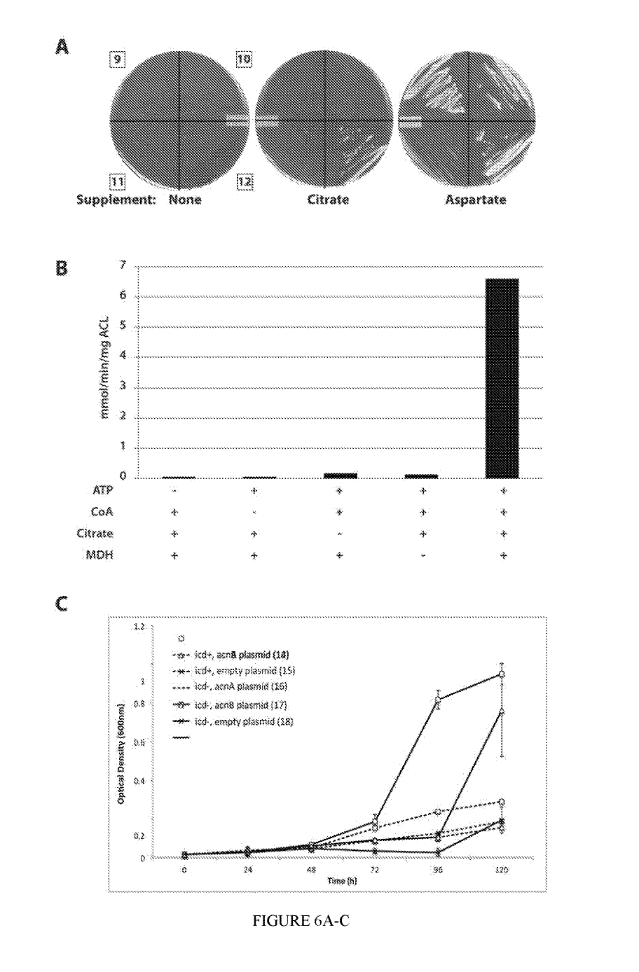

To test these various options for “reverse citrate synthase” activity in vivo, an aspartate auxotrophic E. coli mutant strain was generated, (ΔgltA Δppc Δmdh Δmqo ΔcitE), hereafter referred to as Asp⁻ (FIG. 5). The Asp⁻ strain is deleted of all enzymes that produce the aspartate precursor OAA (ppc, mdh, mqo) and is also deleted of the genes that could have reverse citrate synthase activity (gltA, citE). For the ‘reverse citrate synthase’ assay, the recombinant citrate transporter CitA from Salmonella enterica was also expressed (Shimamoto et al., 1991) (Se CitA), to enable citrate uptake from the medium. This strain should only be able to grow on minimal medium supplemented with citrate if it is able to convert citrate provided in the medium to OAA, an aspartate precursor (Strain 9, FIG. 6A). As expected, overexpression of E. coli citrate synthase gltA did not restore growth on citrate containing plates (Strain 10, FIG. 6A). In addition, it was determined that native expression levels of citrate lyase citDEF were unable to restore growth (Strain 11 FIG. 6A: Asp⁻ strain without citE knockout). This could be due to repression of the citrate lyase operon under aerobic conditions. Instead of overexpressing the citrate lyase operon together with the acetate:CoA ligase, we tested the activity of the more direct ATP-citrate lyase from Chlorobium tepidum (Ct AclAB) (Kim and Tabita, 2006). This route has the same ATP-requirements as the native E. coli route involving citrate lyase and acetate:CoA ligase, but requires overexpression of fewer genes. Ct AclAB was expressed in the Asp⁻ strain and was shows that this heterologous enzyme allowed for growth on citrate-supplemented medium, providing evidence that this enzyme was active in vivo and formed the essential intermediate OAA from citrate (Strain 12, FIG. 6A). The activity of Ct ACL was confirmed in vitro in an enzyme assay using His-tagged protein purified from E. coli (FIG. 6B).

As was the case with malate synthase reversal, use of an ATP-coupled enzyme enabled the initially unfavorable reverse reaction of citrate synthase.

Optimization of the Isocitrate Branchpoint.

After testing the thermodynamically challenging steps of the pathway individually, activity of multiple steps in concert were then tested. First combined overexpression of Ct AclAB and Ec AceA was tested to see if it allowed the Asp⁻ strain to grow on glucose minimal medium supplemented with glyoxylate and succinate. Here, the strain is expected to grow only if glyoxylate and succinate can be condensed to isocitrate, and if that, in turn, can be converted to citrate by the aconitases (via aconitate). Citrate would then act as a substrate for ACL to produce OAA and rescue the aspartate auxotrophy. As a precaution, malate synthase aceB was deleted to prevent loss of glyoxylate to malate. As shown in FIG. 6C (strain 15), extremely slow growth was observed under these conditions. This was hypothesized to be due to isocitrate being drained away from the aconitases (ACN) by isocitrate dehydrogenase (ICD), which competes for the same substrate (see FIG. 5). Thus, the isocitrate branchpoint was tuned to favor the pathway, by i) overexpressing each of the two native E. coli aconitases acnA and acnB, ii) deleting the icd gene (in which case glutamate was provided to the medium), or iii) combining these two modifications. As indicated by the growth rate of the various strains tested on a medium supplemented with glyoxylate and succinate, the metabolic flux was best channeled into the pathway by combining icd deletion and acnA overexpression (strain 13, FIG. 6C).

Assembly of the Full Pathway from Malate and Succinate to Acetyl-CoA and OAA.

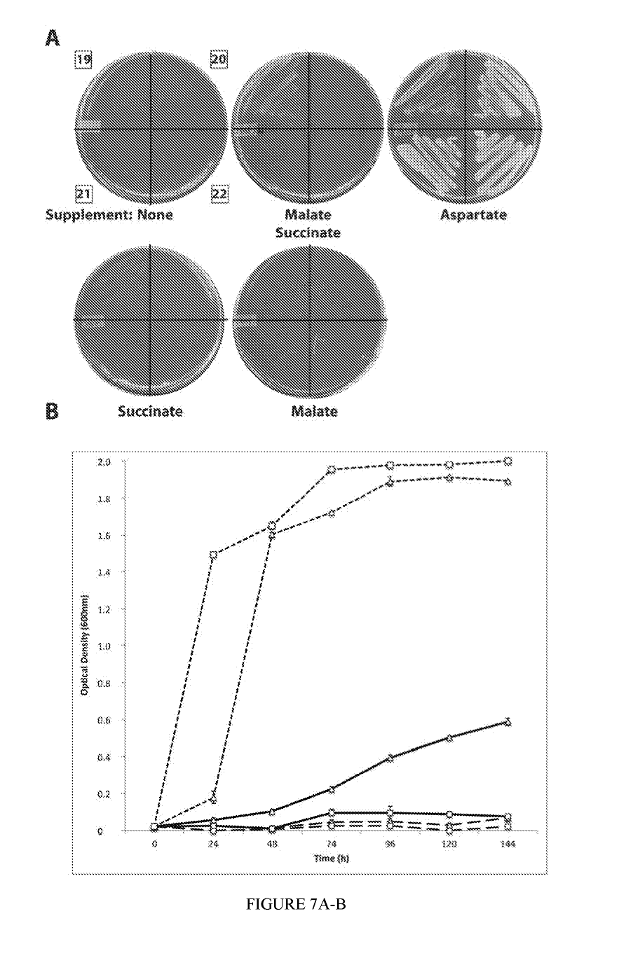

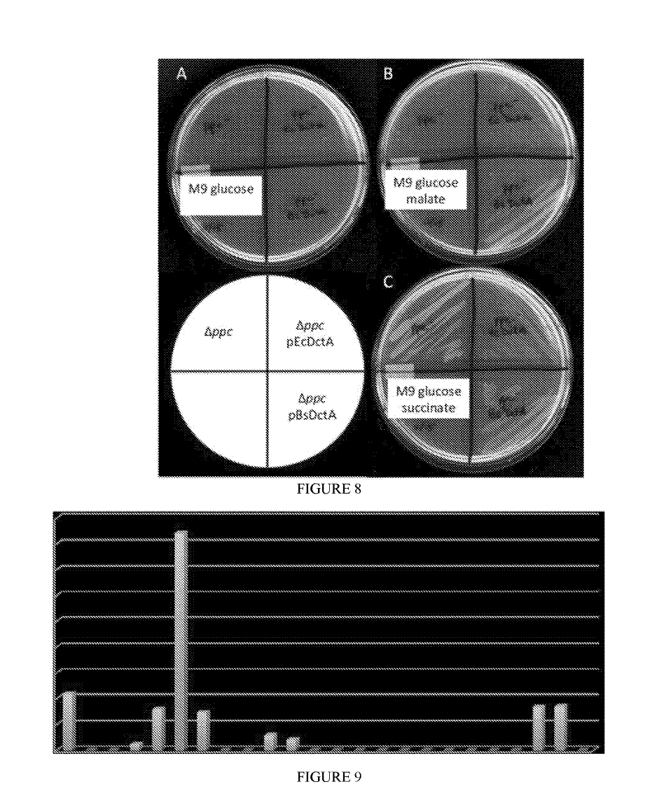

Having identified active enzymes for each step and optimized the critical branchpoint all these features were incorporated into the Asp⁻ strain, and tested whether the full pathway could provide OAA to support growth from malate and succinate Bs dctA, Mc sucCD-2 and Rs mcl1 were overexpressed in the Asp⁻ strain with icd and aceB knockouts, together with Ec aceA, Ec acnA and Ct aclAB. This strain was able to grow on glucose minimal medium supplemented with malate and succinate (Strain 19, FIG. 7A-B). Control strains missing key genes of the pathway (aclAB, or aceA and acnA, or mcl1; Strains 179, 180 and 181 respectively) were not able to grow under these conditions, and growth of the strain containing the full pathway is dependent on the presence of malate and succinate. These results demonstrate a complete in vivo reversal of the glyoxylate pathway from malate and succinate to OAA and two molecules of acetyl-CoA.

In order to test rGS pathway in plants, a plant material that has either null or very low CO₂ fixation. In this case a plant having Rubisco suppressors and/or sbpase mutants were used. An rGS construct was then transformed into these plants.

A plant source that has either suppressed SBPase or Rubisco genes in the Calvin cycle were used for purposes of experimentation only. The Calvin cycle is the primary pathway for photosynthetic carbon fixation, which, in higher plants, is carried out in the chloroplast stroma. This cycle consists of 13 reaction steps catalyzed by 11 different enzymes. SBPase is an enzyme that has only one copy in Arabidopsis.

Sbpase T-DNA insertion lines (SALK_130939) was used at the SBPase locus (AT3G55800) acquired from Arabidopsis Biological Resource Center (ABRC). The loss of function SBPase mutants was severely retarded and the transition to bolting and flowering was much delayed compared with that of wild-type seedlings (Liu et al., 2012). More than 90% of wild-type plants flowered after 5 weeks under the growing conditions compared to more than 10 weeks for 90% of sbp mutant plants. Despite the severe retardation of growth and development, sbp mutant plants are still able to flower and produce seeds under normal growth conditions. Homozygous and heterozygous plant's seeds were used for transformation with the rGS constructs.

Ribulose 1,5-bisphosphate carboxylase/oxygenase (Rubisco; EC 4.1.1.39) is a stromal protein which catalyses two competing reactions of photosynthetic CO₂ fixation and photorespiratory carbon oxidation. In higher plants and green algae, Rubisco is composed of eight small subunits (RBCS) coded for by an RBCS multigene family in the nuclear genome, and eight large subunits (RbcL) coded for by a single RbcL gene. In Arabidopsis, four RBCS members, RBCS1A (At1g67090), RBCS1B (At5g38430), RBCS2B (At5g38420), and RBCS3B (At5g38410), have been identified. Seeds of T-DNA insertion lines for these 4 genes were obtained from Arabidopsis Biological Resource Center (ABRC). A screen was carried out for T-DNA insertion mutants of these RBCS genes, and homozygous mutant lines of RBCS1A and RBCS3B were isolated. The double mutant of these genes was generated by reciprocal crossing and delayed vegetative growth and flowering in these plants was compared to WT.

Another approach was used to suppress the endogenous carbon fixation pathway (CBB cycle) by disrupting the CBB cycle in an inducible fashion. This conditional CBB mutant line can also be transformed with all the genes required for a functional rGS cycle. In this model, the CBB disruption will then be induced in the resulting primary transformants. The transgenic lines that express all the foreign genes, in a balanced way, are expected to survive longer in this CBB disruption. They will thus be easily identified among a large transformant population, and selected for further characterization.

No herbicide targets the CBB cycle. Therefore, in order to disrupt the CBB cycle, the CBB genes were silenced using the artificial microRNA (amiR) strategy. Several amiRs were designed to specifically silence ribulose bisphosphate carboxylase small subunit (RbcS) gene family. In each case, the Web Micro-RNA Designer WMD3 ([http://]wmd3.weigelworld.org/) predicted a number of suitable amiRs that were tested. The expression of these amiRs were placed under the control of an estradiol-inducible promoter. Primary transformants (T0) per amiR were grown to maturity, and T1 seeds collected. From each T1's seeds, 12 seedlings were grown to maturity and seeds collected for segregation analysis. Some were tested for amiR expression and CBB knockout efficiency triggered by estradiol treatment. A successful CBB disruption, triggered by the amiR, was able to show a different phenotype such as flowering defects, resulting in growth arrest, chlorosis etc. Based on these results, 5 amiR lines were selected that can be used for transformation with rGS pathway.

An rGS construct was formed using 11 genes from various sources as described above and set forth in table 3 below:

TABLE 3

| | | | | Transit | |
|---|---|---|---|---|---|
| Gene | Abbr. | Origin | Promoter | Peptide | Terminator |
| Aconitase | ac n | Arabidopsis thanliana | 35s | AT2G28000 | OCS |
| NADP-Malate | mdh | Chlamydomona reinhardtii | 35s | AT1G08490 | ADH1 |
| dehydrogenase | | | | | |
| Fumarase | fumc | Synechocystis sp. PCC 6803 | Mannopine | AT2G28000 | Heat shock |
| | | | Synthase | | |
| Fumarate | frds | Saccharomyces cerevisiae | 35s | AT2G28000 | OCS |
| Reductase | | | | | |
| ATP-Citrate | acl | Homo sapiens | Mannopine | AT4G28660 | UBQ5 |
| Lyase | | | Synthase | | |
| Pyruvate | nifJ | Synechocystis sp. PCC 6803 | 35s | AT1G67090 | ADH |
| oxiodoreductase | | | | | |
| Malate thiokinase | mtkA | Methylococcus capsulatus | 35s | AT1G67090 | ADH |
| Malate thiokinase | mtkB | Methylococcus capsulatus | 35s | AT1G67090 | ADH |
| Malayl-CoA | mcl | Methylobacterium | Mannopine | AT1G10500 | Heat shock |
| | | extorquens | Synthase | | |
| Isocitrtae lyase | IclA | Ralstonia eutropha | 35s | AT1G67090 | OCS |
| Pyruvate | pyc | Lactococcus lactis | Mannopine | AT1G10500 | UBQ5 |
| carboxylase | | | Synthase | | |

pBR6 comprises Aconitase, NADP-Malate dehydrogenase, Fumarase and Fumarase Reductase and all other genes were taken into pDS31. These were transformed into Agrobacterium (LBA 4404) and transformed into WT, SBPase (Heterozygous/Homozygous) and Rubico suppressor lines (double mutants) using floral dip method. Positive transformants were selected on Basta plates (1/2 MS medium) and later screened for DS-Red markers. All selected lines were grown for seed and later screened for phenotypic difference in T1 generation.

Plants were grown on SunGro-Mix #4 in 4-inch-square pots and cultivated in a controlled-environment chamber (Percival Scientific, 1A, USA) at 120 to 140 flmol photons m² s¹ 14 h of light at 21° C., and 10 h of dark at 19° C.

Genotypings and RT-PCR Studies. Genomic DNA was isolated from 11-d-old seedlings of all transgenic lines, WT and mutant lines using C-TAB method or N-AMP PCR lit (Sigma). Total RNA was isolated from 11-d-old seedlings of all transgenic lines using an RNeasy Mini Kit (Qiagen, Valencia, Calif.), according to the manufacturer's instructions. RNA was quantified and evaluated for purity using a Nanodrop Spectrophotometer ND-100 (NanoDrop Technologies, Willington, Del.).

For quantitative two-step RT-PCR, 1 μg of total RNA was reverse-transcribed to first-strand cDNA with the Qiagen cDNA synthesis kit (Qiagen, Hilden, Germany), and those cDNA were subsequently used as a template for qPCR with gene-specific primers. The plant-specific EF4A2 (Atlg54270) gene served as a control for constitutive gene expression.

Certain embodiments of the invention have been described. It will be understood that various modifications may be made without departing from the spirit and scope of the invention. Other embodiments are within the scope of the following claims. Chemoautotrophs, photoautotroph, cyanobacteria overexpress FPK, XPK, tied to non-native promoter.

1. A recombinant microorganism comprising a metabolic pathway for the synthesis of acetyl-CoA and isocitrate from a four-carbon substrate using a pathway comprising one or more polypeptides having malate thiokinase activity, malyl-CoA lyase activity and/or isocitrate lyase activity.

2. The recombinant microorganism of claim 1, wherein the microorganism is a prokaryote or eukaryote.

3-6. (canceled)

7. The recombinant microorganism of claim 1, wherein the polypeptide having malate thiokinase activity is cloned from Methylococcus capsulatus.

8. The recombinant microorganism of claim 1, wherein the polypeptide having malate thiokinase activity comprises a heterodimer of sucC-2 and sucD-2 from Methylcoccus capsulatus.

9. The recombinant microorganism of claim 1, wherein the polypeptide having malate thiokinase activity comprises a sequence that is at least 40% to 100% identical to SEQ ID NO:2 and 4 and converts malate to malyl-coA.

10. The recombinant microorganism of claim 1, wherein the recombinant microorganism is engineered to express or over express a malyl-coA lyase.

11. The recombinant microorganism of claim 1, wherein the polypeptide having malyl-coA lyase activity is cloned from Rhodobacter sphaeroides.

12. The recombinant microorganism of claim 11, wherein the polypeptide having malyl-coA lyase activity comprises a mcl1 from Rhodobacter sphaeroides.

13. The recombinant microorganism of claim 1, wherein the polypeptide having malyl-coA lyase activity comprises a sequence that is at least 40% to 100% identical to SEQ ID NO:8 and converts malyl-coA to glyoxylate.

14. The recombinant microorganism of claim 1, wherein the recombinant microorganism is engineered to express or overexpress an isocitrate lyase.

15. The recombinant microorganism of claim 14, wherein the isocitrate lyase is cloned from E. coli.

16. The recombinant microorganism of claim 15, wherein the isocitrate lyase comprises aceA from E. coli.

17. The recombinant microorganism of claim 1, wherein the polypeptide having isocitrate lyase activity comprises a sequence that is at least 40% to 100% identical to SEQ ID NO:10 and converts glyoxylate and succinate to isocitrate.

18. The recombinant microorganism of claim 1, further comprising expressing or over expressing malate dehydrogenase.

19-20. (canceled)

21. The recombinant microorganism of claim 1, wherein the microorganism is further engineered to express or over express a polypeptide selected from the group consisting of an aconitase, an ATP citrate lyase and a combination thereof.

22. (canceled)

23. The recombinant microorganism of claim 1, further comprising one or more genes selected from the group consisting of atoB, hbd, crt, ter, and adhE2, and wherein the microorganism produces 1-butanol.

24. The recombinant microorganism of claim 1, further comprising one or more enzymes that convert acetyl-CoA to: ethanol, fatty acid or isoprenoid.

25. The recombinant microorganism of claim 1, further comprising a CO₂ fixation pathway.

26. The recombinant microorganism of claim 23, wherein the microorganism further comprises pta.

27. The recombinant microorganism of claim 1, wherein the microorganism further comprises one or more knockouts selected from the group consisting of: Δicd, ΔgltA, ΔcitDEF, Δmdh/mqo, Δppc, ΔadhE, Δack, a homolog of any of the foregoing, and any combination thereof.

29. A cell-free system for converting a 4-carbon substrate to isocitrate and two acetyl-CoAs comprising ATP and CoA and:
(i) an enzyme the converts malate to malyl-CoA;
(ii) an enzyme the converts malyl-CoA to glycosylate and acetyl-CoA;
(iii) an enzyme that converts isocitrate to citrate; and
(iv) an enzyme that converts citrate to oxaloacetate.

30. The cell-free system of claim 29, wherein each of (i)-(iv) are obtained from a different microorganism by expressing the microorganism and disrupting the organism or isolating the enzyme from the organism.

31. The cell-free system of claim 30, wherein the different microorganism are recombinantly engineered to express an enzyme of (i)-(iv).

32. A recombinant microorganism for producing 1-butanol, wherein the microorganism comprises:
(i) an enzyme the converts malate to malyl-CoA;
(ii) an enzyme the converts malyl-CoA to glycosylate and acetyl-CoA;
(iii) an enzyme that converts isocitrate to citrate;
(iv) an enzyme that converts citrate to oxaloacetate;
(v) an enzyme that converts acetyl-CoA to acetoacetyl-CoA, and at least one enzyme that converts (a) acetoacetyl-CoA to (R)- or (S)-3-hydroxybutyryl-CoA and (R)- or (S)-3-hydroxybutyryl-CoA to crotonyl-CoA,
(vi) an enzyme that converts crotonyl-CoA to butyryl-CoA; and
(vii) an enzyme that converts butyryl-CoA to butyraldehyde and butyraldehyde to 1-butanol.

33. The recombinant microorganism of claim 32, wherein the microorganism comprises an expression profile selected from the group consisting of:
(a) Mtk, Mcl, aceA (or icl), acnAB, Acl, AtoB, Hbd, Crt, Ter, BldH, and YqhD,
(b) Mtk, Mcl, aceA (or icl), acnAB, Acl, AtoB, Hbd, Crt, Ter, and AdhE2;
(c) Mtk, Mcl, aceA (or icl), acnAB, Ad, AtoB, Hbd, Crt, ccr, BldH, and YqhD, and
(d) Mtk, Mcl, aceA (or icl), acnAB, Acl, AtoB, Hbd, Crt, ccr, and AdhE2.

34. A recombinant plant engineered to express one or more polypeptides having activity selected form the group consisting of malate thiokinase activity, malyl-CoA lyase activity, pyruvate:ferrodoxin oxidoreductase activity and fumarase reductase activity and wherein the recombinant plant produces more acetyl-CoA compared to a wild-type of parental plant.

35. The recombinant plant of claim 34, wherein the plant exhibits at least one characteristic selected from the group consisting of: (a) increased biomass compared to a wild-type or parental plant, (b) improved CO₂ utilization compared to a wild-type or parental plant, (c) reduced or no photorespiration compared to a wild-type or parental plant, (d) improved photosynthetic efficiency compared to a wild-type or parental plant, (e) improved vegetative biomass compared to a parental or wild-type plant, (f) increased seed production compared to a parental or wild-type plant, (g) improved harvest index compared to a parental or wild-type plant, and (h) any combination of (a)-(g).

36-41. (canceled)

42. The recombinant plant of claim 34, wherein the plant has a mutant sbpase gene.

43. The recombinant plant of claim 34, wherein the plant comprises a reduced expression or activity or lacks activity of RuBisco.

44. The recombinant plant of claim 34, wherein the plant is a crop plant for oil, biofuel, chemicals, animal feed, cereal or forage.

45-49. (canceled)

50. A recombinant plant of claim 34, wherein the plant expresses or over expresses enzymes selected from the group consisting of aconitase, NADP-malate dehydrogenase, fumarase, fumarate reductase, ATP-citrate lyase, pyrufate:ferrodoxin oxidoreductase, malate thiokinase, malyl-CoA lyase, isocitrate lyase, pyruvate carboxylase and any combination thereof.

51. The recombinant plant of claim 50, wherein the plant comprises a genotype selected from the group consisting of acn, mdh, fumc, frd, acl, nifJ, mtkA, mtkB, mcl, icl, pyc and genes of any combination thereof.

52-67. (canceled)


SEQUENCE LISTING (106 sequences)

SEQ ID NO: 1 - DNA, 1170 bp, Methylococcus capsulatus
  CDS (1)..(1170)
gtgaatatccatgagtaccaggccaaggagctgctcaagacctatggcgtgcccgtgcccgacggcgccgttgcctattccgacgcgcaggccgccagcgtcgccgaggagatcggcggcagccgctgggtggtcaaggcgcagatccatgccggcggtcgcggcaaggccgggggcgtaaaggtcgcccactccatcgaggaagtccgccaatacgccgacgccatgctcggcagccacctcgtcacccatcagaccggcccgggaggctcgctggttcagcgtctgtgggtggaacaggccagccatatcaaaaaggaatactacctgggcttcgtgatcgatcgcggcaatcaacgcatcaccctgatcgcctccagcgagggcggcatggaaatcgaggaagtcgcaaaggaaaccccggagaaaatcgtcaaggaagtcgtcgatccggccataggcctgctggacttccagtgccgcaaggtcgccacggcgatcggcctgaaaggcaaactgatgccccaggccgtcaggctgatgaaggccatctaccgctgcatgcgcgacaaagatgccctgcaggccgaaatcaatcctctggccatcgtgggcgaaagcgacgaatcgctcatggtcctggatgccaagttcaacttcgacgacaacgccctgtaccggcagcgcaccatcaccgagatgcgcgacctggccgaggaagacccgaaagaggtcgaagcctccggccacggtctcaattacatcgccctcgacggcaacatcggctgcatcgtcaatggcgccggcctcgccatggcttcgctcgacgccatcaccctgcatggcggccgtccggccaacttcctcgacgtgggcggcggcgcctcccccgagaaggtcaccaatgcctgccgcatcgtactggaagatcccaacgtccgctgcatcctggtcaacatctttgccggcatcaaccgctgtgactggatcgccaagggcctgatccaggcctgcgacagcctgcagatcaaggtgccgctgatcgtgcgcctggccgggacgaacgtcgacgagggccgcaagatcctggccgaatccggcctctccttcatcaccgcggaaaatctggacgacgcggccgccaaggccgtcgccatcgtcaagggataa

SEQ ID NO: 2 - protein, 389 aa, Methylococcus capsulatus
VNIHEYQAKELLKTYGVPVPDGAVAYSDAQAASVAEEIGGSRWVVKAQIHAGGRGKAGGVKVAHSIEEVRQYADAMLGSHLVTHQTGPGGSLVQRLWVEQASHIKKEYYLGFVIDRGNQRITLIASSEGGMEIEEVAKETPEKIVKEVVDPAIGLLDFQCRKVATAIGLKGKLMPQAVRLMKAIYRCMRDKDALQAEINPLAIVGESDESLMVLDAKFNFDDNALYRQRTITEMRDLAEEDPKEVEASGHGLNYIALDGNIGCIVNGAGLAMASLDAITLHGGRPANFLDVGGGASPEKVTNACRIVLEDPNVRCILVNIFAGINRCDWIAKGLIQACDSLQIKVPLIVRLAGTNVDEGRKILAESGLSFITAENLDDAAAKAVAIVKG

SEQ ID NO: 3 - DNA, 903 bp, Methylococcus capsulatus
  CDS (1)..(903)
atgagcgtattcgttaacaagcactccaaggtcatcttccagggcttcaccggcgagcacgccaccttccacgccaaggacgccatgcggatgggcacccgggtggtcggcggtgtcacccctggcaaaggcggcacccgccatcccgatcccgaactcgctcatctgccggtgttcgacaccgtggctgaagccgtggccgccaccggcgccgacgtctccgccgtgttcgtgccgccgcccttcaatgcggacgcgttgatggaagccatagacgccggcatccgggtcgccgtgaccatcgccgacggcatcccggtacacgacatgatccgactgcagcgctaccgggtgggtaaggattccatcgtgatcggaccgaacacccccggcatcatcacgccgggcgagtgcaaggtgggcatcatgccttcgcacatttacaagaagggcaacgtcggcatcgtgtcgcgctccggcaccctcaattacgaggcgacggaacagatggccgcgcttgggctgggcatcaccacctcggtcggtatcggcggtgaccccatcaacggaaccgatttcgtcactgtcctgcgcgccttcgaagccgacccggaaaccgagatcgtggtgatgatcggcgaaatcggcggcccccaggaagtcgccgccgcccgctgggccaaggaaaacatgacaaagccggtcatcggcttcgtcgcaggccttgccgcaccgaccggccgacgcatgggccatgccggcgccatcatctccagcgaggccgacaccgccggagccaagatggacgccatggaagccttggggctgtatgtcgcccgcaacccggcacagatcggccagaccgtgctacgcgccgcgcaggaacacggaatcagattctga

SEQ ID NO: 4 - protein, 300 aa, Methylococcus capsulatus
MSVFVNKHSKVIFQGFTGEHATFHAKDAMRMGTRVVGGVTPGKGGTRHPDPELAHLPVFDTVAEAVAATGADVSAVFVPPPFNADALMEAIDAGIRVAVTIADGIPVHDMIRLQRYRVGKDSIVIGPNTPGIITPGECKVGIMPSHIYKKGNVGIVSRSGTLNYEATEQMAALGLGITTSVGIGGDPINGTDFVTVLRAFEADPETEIVVMIGEIGGPQEVAAARWAKENMTKPVIGFVAGLAAPTGRRMGHAGAIISSEADTAGAKMDAMEALGLYVARNPAQIGQTVLRAAQEHGIRF

SEQ ID NO: 5 - DNA, 939 bp, Escherichia coli
  CDS (1)..(939)
atgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcgcgtaaaccgggtatggatcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 6 - protein, 312 aa, Escherichia coli
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVAKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 7 - DNA, 957 bp, Rhodobacter sphaeroides
  CDS (1)..(957)
atgagcttccgtcttcagcccgcgccgcctgcccgtccgaaccgctgccagctgttcggccccggctcccggcccgcgctgttcgagaagatggcggcctccgcggcggacgtgatcaacctcgatctcgaggattcggtggcgcccgacgacaaggcgcaggcccgcgcgaacatcatcgaggcgatcaacgggctcgactggggccgcaagtatctctcggtccgcatcaacggtctggatacgcccttctggtatcgcgatgtcgtggacctgctcgaacaggccggcgaccggctcgaccagatcatgatcccgaaggttggctgcgcggcggatgtctatgcggtcgatgctctggtcacggccatcgagcgcgccaagggccgcaccaaacccctgagcttcgaggtcatcatcgaatcggccgcgggcatcgcccatgtcgaggaaatcgcggcctcctcgccgcgcctgcaggccatgagcctcggcgccgccgatttcgcagcctcgatggggatgcagacgacaggtatcggcggcacgcaggaaaactactacatgttgcatgacgggcagaagcactggtcggacccgtggcactgggcgcaggcggccatcgtggcggcctgccggacccacgggatcctgcccgtggacggcccgttcggcgatttttccgacgatgagggcttccgcgcgcaggcccgccgctcggccactctgggcatggtgggcaaatgggccatacatcccaaacaggtggccctcgcgaacgaagttttcaccccttccgagacggccgtgaccgaagcgcgcgagatcctcgcggcgatggatgcagccaaggcgaggggcgagggcgccacggtctacaagggaagacttgttgacatcgcgtccatcaaacaggcagaagtgatcgtaaggcaggcagaaatgatctcggcctga

SEQ ID NO: 8 - protein, 318 aa, Rhodobacter sphaeroides
MSFRLQPAPPARPNRCQLFGPGSRPALFEKMAASAADVINLDLEDSVAPDDKAQARANIIEAINGLDWGRKYLSVRINGLDTPFWYRDVVDLLEQAGDRLDQIMIPKVGCAADVYAVDALVTAIERAKGRTKPLSFEVIIESAAGIAHVEEIAASSPRLQAMSLGAADFAASMGMQTTGIGGTQENYYMLHDGQKHWSDPWHWAQAAIVAACRTHGILPVDGPFGDFSDDEGFRAQARRSATLGMVGKWAIHPKQVALANEVFTPSETAVTEAREILAAMDAAKARGEGATVYKGRLVDIASIKQAEVIVRQAEMISA

SEQ ID NO: 9 - DNA, 1305 bp, Escherichia coli
  CDS (1)..(1305)
atgaaaacccgtacacaacaaattgaagaattacagaaagagtggactcaaccgcgttgggaaggcattactcgcccatacagtgcggaagatgtggtgaaattacgcggttcagtcaatcctgaatgcacgctggcgcaactgggcgcagcgaaaatgtggcgtctgctgcacggtgagtcgaaaaaaggctacatcaacagcctcggcgcactgactggcggtcaggcgctgcaacaggcgaaagcgggtattgaagcagtctatctgtcgggatggcaggtagcggcggacgctaacctggcggccagcatgtatccggatcagtcgctctatccggcaaactcggtgccagctgtggtggagcggatcaacaacaccttccgtcgtgccgatcagatccaatggtccgcgggcattgagccgggcgatccgcgctatgtcgattacttcctgccgatcgttgccgatgcggaagccggttttggcggtgtcctgaatgcctttgaactgatgaaagcgatgattgaagccggtgcagcggcagttcacttcgaagatcagctggcgtcagtgaagaaatgcggtcacatgggcggcaaagttttagtgccaactcaggaagctattcagaaactggtcgcggcgcgtctggcagctgacgtgacgggcgttccaaccctgctggttgcccgtaccgatgctgatgcggcggatctgatcacctccgattgcgacccgtatgacagcgaatttattaccggcgagcgtaccagtgaaggcttcttccgtactcatgcgggcattgagcaagcgatcagccgtggcctggcgtatgcgccatatgctgacctggtctggtgtgaaacctccacgccggatctggaactggcgcgtcgctttgcacaagctatccacgcgaaatatccgggcaaactgctggcttataactgctcgccgtcgttcaactggcagaaaaacctcgacgacaaaactattgccagcttccagcagcagctgtcggatatgggctacaagttccagttcatcaccctggcaggtatccacagcatgtggttcaacatgtttgacctggcaaacgcctatgcccagggcgagggtatgaagcactacgttgagaaagtgcagcagccggaatttgccgccgcgaaagatggctataccttcgtatctcaccagcaggaagtgggtacaggttacttcgataaagtgacgactattattcagggcggcacgtcttcagtcaccgcgctgaccggctccactgaagaatcgcagttctaa

SEQ ID NO: 10 - protein, 434 aa, Escherichia coli
MKTRTQQIEELQKEWTQPRWEGITRPYSAEDVVKLRGSVNPECTLAQLGAAKMWRLLHGESKKGYINSLGALTGGQALQQAKAGIEAVYLSGWQVAADANLAASMYPDQSLYPANSVPAVVERINNTFRRADQIQWSAGIEPGDPRYVDYFLPIVADAEAGFGGVLNAFELMKAMIEAGAAAVHFEDQLASVKKCGHMGGKVLVPTQEAIQKLVAARLAADVTGVPTLLVARTDADAADLITSDCDPYDSEFITGERTSEGFFRTHAGIEQAISRGLAYAPYADLVWCETSTPDLELARRFAQAIHAKYPGKLLAYNCSPSFNWQKNLDDKTIASFQQQLSDMGYKFQFITLAGIHSMWFNMFDLANAYAQGEGMKHYVEKVQQPEFAAAKDGYTFVSHQQEVGTGYFDKVTTIIQGGTSSVTALTGSTEESQF

SEQ ID NO: 11 - DNA, 25 bp, Artificial Sequence
  Forward primer gltA
gttgatgtgcgaaggcaaatttaag

SEQ ID NO: 12 - DNA, 25 bp, Artificial Sequence
  Reverse Primer gltA
aggcatataaaaatcaacccgccat

SEQ ID NO: 13 - DNA, 24 bp, Artificial Sequence
  Forward Primer prpC
gtattcgacagccgatgcctgatg

SEQ ID NO: 14 - DNA, 24 bp, Artificial Sequence
  Reverse Primer prpC
ctttgatcattgcggtcagcacct

SEQ ID NO: 15 - DNA, 20 bp, Artificial Sequence
  Forward Primer mdh
ttcttgcttagccgagcttc

SEQ ID NO: 16 - DNA, 20 bp, Artificial Sequence
  Reverse Primer mdh
gggcattaatacgctgtcgt

SEQ ID NO: 17 - DNA, 24 bp, Artificial Sequence
  Forward Primer mqo
gactgctgccgtcaggtcaatatg

SEQ ID NO: 18 - DNA, 24 bp, Artificial Sequence
  Reverse Primer mqo
ctccaccccgtaggttggataagg

SEQ ID NO: 19 - DNA, 22 bp, Artificial Sequence
  Forward Primer ppc
acctttggtgttacttggggcg

SEQ ID NO: 20 - DNA, 22 bp, Artificial Sequence
  Reverse Primer ppc
taccgggatcaaccacagcgaa

SEQ ID NO: 21 - DNA, 24 bp, Artificial Sequence
  Forward Primer aceB
ctatttcccgcacaatgatccgca

SEQ ID NO: 22 - DNA, 24 bp, Artificial Sequence
  Reverse Primer aceB
cttcaatacccgctttcgcctgtt

SEQ ID NO: 23 - DNA, 22 bp, Artificial Sequence
  Forward Primer citE
gcgactgaaacgctatgccgaa

SEQ ID NO: 24 - DNA, 22 bp, Artificial Sequence
  Reverse Primer citE
ttcagttcgccgctctgtacca

SEQ ID NO: 25 - DNA, 19 bp, Artificial Sequence
  Forward Primer icd
gtttacccggctgggttaa

SEQ ID NO: 26 - DNA, 21 bp, Artificial Sequence
  Reverse Primer icd
agtcacgatcgttagcaattg

SEQ ID NO: 27 - DNA, 1200 bp, Methylococcus capsulatus
  CDS (1)..(1200)
atggatattcatgaatatcaagctaaggaaattttggctaattttggagttgatattcctcctggagctttggcttattctcctgaacaagctgcttatagagctagagaaattggaggagatagatgggttgttaaggctcaagttcatgctggaggaagaggaaaggctggaggagttaaggtttgttcttctgatgctgaaattcaagaaacttgtgaaaatttgtttggaagaaagttggttactcatcaaactggacctgaaggaaagggaatttatagagtttatgttgaaggagctgttcctattgaaagagaaatttatttgggatttgttttggatagatcgtctcaaagagttatgattgttgcttctgctgaaggaggaatggaaattgaagaaatttctgctgaaaagcctgattctattgttagagctactgttgaacctgctgttggattgcaagattttcaatgtagacaaattgcttttaagttgggaattgatcctgctttgactgctagaatggttagaactttgcaaggatgttatcaagctttttctgaatatgatgctactatggttgaaattaatcctttggttattactggagataatagaattttggctttggatgctaagatgacttttgatgataatgctttgtttagacatcctcatatttctgaattgagagataagtctcaagaagatcctagagaatctagggctgctgatagaggattgtcttatgttggattggatggaaatattggatgtattgttaatggagctggattggctatggctactatggatactattaagttggctggaggagaacctgctaattttttggatattggaggaggagctactcctgaaagagttgctaaggcttttagattggttatgtctgattctaatgttcaagctgttttggttaatatttttgctggaattaatagatgtgattgggttgctgaaggagttgttcaagctttgaaggaagttcaagttgaagttcctgttattgttagattggctggaactaatgttgaagaaggacaaaagattttggctaagtctggattgcctattattagagctagaactttgatggaagctgctgaaagagctgttggagcttggcaaaatgatttgtctgaaaatactattgttagagctgttcaataa

SEQ ID NO: 28 - protein, 399 aa, Methylococcus capsulatus
MDIHEYQAKEILANFGVDIPPGALAYSPEQAAYRAREIGGDRWVVKAQVHAGGRGKAGGVKVCSSDAEIQETCENLFGRKLVTHQTGPEGKGIYRVYVEGAVPIEREIYLGFVLDRSSQRVMIVASAEGGMEIEEISAEKPDSIVRATVEPAVGLQDFQCRQIAFKLGIDPALTARMVRTLQGCYQAFSEYDATMVEINPLVITGDNRILALDAKMTFDDNALFRHPHISELRDKSQEDPRESRAADRGLSYVGLDGNIGCIVNGAGLAMATMDTIKLAGGEPANFLDIGGGATPERVAKAFRLVMSDSNVQAVLVNIFAGINRCDWVAEGVVQALKEVQVEVPVIVRLAGTNVEEGQKILAKSGLPIIRARTLMEAAERAVGAWQNDLSENTIVRAVQ

SEQ ID NO: 29 - DNA, 891 bp, Methylococcus capsulatus
  CDS (1)..(891)
atgtctatttttattgatagagaaactcctgttattgttcaaggaattactggaaagatggctagatttcatactgctgatatgattgcttatggaactaatgttgttggaggagttgttcctggaaagggaggacaaactgttgaaggagttcctgtttttgatactgttgaagaagctgttgaaagaactggagctgaagcttctttggtttttgttcctcctccttttgctgctgattctattatggaagctgctgatgctggaattagatattgtgtttgtattactgatggaatacctgctcaagatatgattagagttaagagatatatgtatagatatcctagagaaagaagaatggttttgactggacctaattgtgctggaactatttctcctggaaaggctttgttgggaattatgcctggacatatttatttgcctggacctgttggaattattggaagatcgggaactttgggatatgaagctgctgctcaattgaaggaacatggaattggagtttctacttctgttggaattggaggagatcctattaatggatcttcttttaaggatattttgcatagatttgaacaagatgatgaaactcatgttatttgtatgattggagaaattggaggacctcaagaagctgaagctgctgcttatattagagatcatgtttctaagcctgttattgcttatgttgctggattgactgctcctaagggaagaactatgggacatgctggagctattatttctgcttttggagaatctgcttctgaaaaggttgaaattttgactgctgctggagttactgttgctcctaatcctgctgttattggagatactattgctagagttatgagagaagctgcttaa

SEQ ID NO: 30 - protein, 296 aa, Methylococcus capsulatus
MSIFIDRETPVIVQGITGKMARFHTADMIAYGTNVVGGVVPGKGGQTVEGVPVFDTVEEAVERTGAEASLVFVPPPFAADSIMEAADAGIRYCVCITDGIPAQDMIRVKRYMYRYPRERRMVLTGPNCAGTISPGKALLGIMPGHIYLPGPVGIIGRSGTLGYEAAAQLKEHGIGVSTSVGIGGDPINGSSFKDILHRFEQDDETHVICMIGEIGGPQEAEAAAYIRDHVSKPVIAYVAGLTAPKGRTMGHAGAIISAFGESASEKVEILTAAGVTVAPNPAVIGDTIARVMREAA

SEQ ID NO: 31 - DNA, 1197 bp, Arabidopsis thaliana
  CDS (1)..(1197)
atggctaagattttggaaggacctgctatgaagttgtttaataagtggggaatacctgttcctaattatgttgttattatggatcctaagagattggctcaattgggagaagctaataagtggttgagagaatctaagttggttgttaaggctcatgaagctattggaggaagatttaagttgggattggttaagattggattgaatttggatgaagctattcaagcttctagggaaatgttgggagctaaggttggaactgctgaagttagacaagttattgttgctgaaatgttggatcatgatgctgaattttatgtttctattattggaaatagagatggagctgaattgttgatttctaagtatggaggagttgatattgaagataattgggattctgttagaagaatacaaattcctttggatgaacatcctactattgaacaattgactgctttggctaaggaagctggatttgaaggagaaattgctgaaagagttggaaagatttgttctaggttggttttgtgttttgataatgaagatgctcaatctattgaaattaatcctttggttattagaaagtctgatatgagatttgctgctttggatgctgttatgaatgttgattgggatgctagatttagacatgctgattgggattttaagcctgtttctgaaattggaagaccttttactgaagctgaacaacaaattatggatattgattctaggattaagggatctgttaagtttgttgaagttcctggaggagaaattgctttgttgactgctggaggaggagcttctgttttttatgctgatgctgttgttgctagaggaggaactattgctaattatgctgaatattctggagatcctcctgattgggctgttgaagctttgactgaaactatttgtagattgcctaatattaagcatattattgttggaggagctattgctaattttactgatgttaaggctactttttctggaattattaatggattgagagaatctaagtctaagggatatttggaaggagttaagatttgggttagaagaggaggacctaatgaagctcaaggattggctgctattagaaagttgcaagaagaaggatttgatattcatgtttatgatagatcaatgcctatgactgatattgttgatttggctttgaagtcttaa

SEQ ID NO: 32 - protein, 398 aa, Arabidopsis thaliana
MAKILEGPAMKLFNKWGIPVPNYVVIMDPKRLAQLGEANKWLRESKLVVKAHEAIGGRFKLGLVKIGLNLDEAIQASREMLGAKVGTAEVRQVIVAEMLDHDAEFYVSIIGNRDGAELLISKYGGVDIEDNWDSVRRIQIPLDEHPTIEQLTALAKEAGFEGEIAERVGKICSRLVLCFDNEDAQSIEINPLVIRKSDMRFAALDAVMNVDWDARFRHADWDFKPVSEIGRPFTEAEQQIMDIDSRIKGSVKFVEVPGGEIALLTAGGGASVFYADAVVARGGTIANYAEYSGDPPDWAVEALTETICRLPNIKHIIVGGAIANFTDVKATFSGIINGLRESKSKGYLEGVKIWVRRGGPNEAQGLAAIRKLQEEGFDIHVYDRSMPMTDIVDLALKS

SEQ ID NO: 33 - DNA, 1248 bp, Chlamydomonas reinhardtii
  CDS (1)..(1248)
atggctttgaatatgaagcaacaacaagctggattgtctaggaaggctgctagatcggtttcttctagggctcctgttgttgttagagctgttgctgctcctgttgctcctgctgctgaagctgaagctaagaaggcttatggagtttttagattgtcttatgatactcaaaatgaagatgcttctttgactagatcgtggaagaagactgttaaggttgctgttactggagcttctggaaatattgctaatcatttgttgtttatgttggcttctggagaagtttatggaaaggatcaacctattgctttgcaattgttgggatctgaaagatcgaaggaagctttggaaggagttgctatggaattggaagattctttgtatcctttgttgagagaagtttctattggaactgatccttatgaagtttttggagatgctgattgggctttgatgattggagctaagcctagaggacctggaatggaaagagctgatttgttgcaacaaaatggagaaatttttcaagttcaaggaagagctttgaatgaatctgcttctaggaattgtaaggttttggttgttggaaatccttgtaatactaatgctttgattgctatggaaaatgctcctaatattcctagaaagaattttcatgctttgactagattggatgaaaatagagctaagtgtcaattggctttgaagtctggaaagttttatacttctgtttctaggatggctatttggggaaatcattctactactcaagttcctgattttgttaatgctagaattggaggattgcctgctcctgatgttattagagatatgaagtggtttagagaagaatttactcctaaggttgctttgagaggaggagctttgattaagaagtggggaagatcgtctgctgcttctactgctgtttctgttgctgatgctattagagctttggttgttcctactgctcctggagattgtttttctactggagttatttctgatggaaatccttatggagttagagaaggattgattttttcttttccttgtagatcgaagggagatggagattatgaaatttgtgataattttattgttgatgaatggttgagagctaagattagagcttctgaagatgaattgcaaaaggaaaaggaatgtgtttctcatttgattggaatgatgggaggatcttgtgctttgagaggagctgaagatactactgttcctggagaaaattaa

SEQ ID NO: 34 - protein, 415 aa, Chlamydomonas reinhardtii
MALNMKQQQAGLSRKAARSVSSRAPVVVRAVAAPVAPAAEAEAKKAYGVFRLSYDTQNEDASLTRSWKKTVKVAVTGASGNIANHLLFMLASGEVYGKDQPIALQLLGSERSKEALEGVAMELEDSLYPLLREVSIGTDPYEVFGDADWALMIGAKPRGPGMERADLLQQNGEIFQVQGRALNESASRNCKVLVVGNPCNTNALIAMENAPNIPRKNFHALTRLDENRAKCQLALKSGKFYTSVSRMAIWGNHSTTQVPDFVNARIGGLPAPDVIRDMKWFREEFTPKVALRGGALIKKWGRSSAASTAVSVADAIRALVVPTAPGDCFSTGVISDGNPYGVREGLIFSFPCRSKGDGDYEICDNFIVDEWLRAKIRASEDELQKEKECVSHLIGMMGGSCALRGAEDTTVPGEN

SEQ ID NO: 35 - DNA, 1401 bp, Synechocystis PCC6803
  CDS (1)..(1401)
atggttaattctcatagattggaaactgattctatgggatctttggaagttcaagctgatagattgtggggagctcaaactcaaagatcgttgatgttttttgatattggatctgatgttatgcctcctgatttgattagagcttttgctattttgaagaaggctgctgctattactaatcaagatttgggaaagttgcctgctgataaggctgaattgattattactgctgctgatgaaattattgctggacaatggttggatcattttcctttgagaatttggcaaactggatctggaactcaaactaatatgaatgttaatgaagttattgctaatagagctattgctatttgtggaggagaattgggatctaagaatcctattcatcctaatgatcatgttaatatgtctcaatcttctaatgatacttttcctactgctatgcatattgctgctgttgctggattgcaaactaagttgattccttctttgcaagctttgagagattctttgaatgaaaaggctgaatgttttgctggaattactaagattggaagaactcatttgatggatgctgttcctttgactttgggacaagaattttctggatatgttgctcaattggatcaaggattgactcaaattaattattgtttgcctggattgttggaattggctttgggaggaactgctgttggaactggattgaatagtcatcctcaatttgctaagaaggttgctgaagaaattgctcaattgactggatatacttttatttctgctcctaataagtttgctgctttggctggacatgaagctattgcttttgcttctggagttttgaagtctattgctgcttctttgatgaagattgctaatgatttgagatggatgggatctggacctagatgtggattgggagaattggctttgcctgctaatgaacctggatcttctattatgcctggaaaggttaatcctactcaatgtgaagctatgactatggtttgtgttcaagttatgggaaatgatgctactattggatttgctgcttctcaaggaaattttgaattgaatgtttttaagcctgttattattcataattttttgcattctttgcatttgttgtctgatgcttgtgcttcttttagacaacatttggttgttggattgcaagttaatgaatctaaggttaaggattttttggatacttctttgatgttggttactgctttgaatcctcatattggatatgataatgctgctttggttgctaagactgcttttgctcaaggaattactttgaagcaagctgctgttgatttgggattgttgactcctgctcaatttgatgcttgggttgttcctgaacaaatgattgctcctattgctgattaa

SEQ ID NO: 36 - protein, 466 aa, Synechocystis PCC6803
MVNSHRLETDSMGSLEVQADRLWGAQTQRSLMFFDIGSDVMPPDLIRAFAILKKAAAITNQDLGKLPADKAELIITAADEIIAGQWLDHFPLRIWQTGSGTQTNMNVNEVIANRAIAICGGELGSKNPIHPNDHVNMSQSSNDTFPTAMHIAAVAGLQTKLIPSLQALRDSLNEKAECFAGITKIGRTHLMDAVPLTLGQEFSGYVAQLDQGLTQINYCLPGLLELALGGTAVGTGLNSHPQFAKKVAEEIAQLTGYTFISAPNKFAALAGHEAIAFASGVLKSIAASLMKIANDLRWMGSGPRCGLGELALPANEPGSSIMPGKVNPTQCEAMTMVCVQVMGNDATIGFAASQGNFELNVFKPVIIHNFLHSLHLLSDACASFRQHLVVGLQVNESKVKDFLDTSLMLVTALNPHIGYDNAALVAKTAFAQGITLKQAAVDLGLLTPAQFDAWVVPEQMIAPIAD

SEQ ID NO: 37 - DNA, 3429 bp, Saccharomyces cerevisiae
  CDS (1)..(3429)
atggttgatggaagatcgtctgcttctattgttgctgttgatcctgaaagagctgctagagaaagagatgctgctgctagagctttgttgcaagattctcctttgcatactactatgcaatatgctacttctggattggaattgactgttccttatgctttgaaggttgttgcttctgctgatacttttgatagagctaaggaagttgctgatgaagttttgagatgtgcttggcaattggctgatactgttttgaatagttttaatcctaattctgaagtttctttggttggaagattgcctgttggacaaaagcatcaaatgtctgctcctttgaagagagttatggcttgttgtcaaagagtttataattcttctgctggatgttttgatccttctactgctcctgttgctaaggctttgagagaaattgctttgggaaaggaaagaaataatgcttgtttggaagctttgactcaagcttgtactttgcctaattcttttgttattgattttgaagctggaactatttctaggaagcatgaacatgcttctttggatttgggaggagtttctaagggatatattgttgattatgttattgataatattaatgctgctggatttcaaaatgttttttttgattggggaggagattgtagagcttctggaatgaatgctagaaatactccttgggttgttggaattactagacctccttctttggatatgttgcctaatcctcctaaggaagcttcttatatttctgttatttctttggataatgaagctttggctacttctggagattatgaaaatttgatttatactgctgatgataagcctttgacttgtacttatgattggaagggaaaggaattgatgaagccttctcaatctaatattgctcaagtttctgttaagtgttattctgctatgtatgctgatgctttggctactgcttgttttattaagagagatcctgctaaggttagacaattgttggatggatggagatatgttagagatactgttagagattatagagtttatgttagagaaaatgaaagagttgctaagatgtttgaaattgctactgaagatgctgaaatgagaaagagaagaatttctaatactttgcctgctagagttattgttgttggaggaggattggctggattgtctgctgctattgaagctgctggatgtggagctcaagttgttttgatggaaaaggaagctaagttgggaggaaattctgctaaggctacttctggaattaatggatggggaactagagctcaagctaaggcttctattgttgatggaggaaagtattttgaaagagatacttataagtctggaattggaggaaatactgatcctgctttggttaagactttgtctatgaagtctgctgatgctattggatggttgacttctttgggagttcctttgactgttttgtctcaattgggaggacattctaggaagagaactcatagagctcctgataagaaggatggaactcctttgcctattggatttactattatgaagactttggaagatcatgttagaggaaatttgtctggaagaattactattatggaaaattgttctgttacttctttgttgtctgaaactaaggaaagacctgatggaactaagcaaattagagttactggagttgaatttactcaagctggatctggaaagactactattttggctgatgctgttattttggctactggaggattttctaatgataagactgctgattctttgttgagagaacatgctcctcatttggttaattttcctactactaatggaccttgggctactggagatggagttaagttggctcaaagattgggagctcaattggttgatatggataaggttcaattgcatcctactggattgattaatcctaaggatcctgctaatcctactaagtttttgggacctgaagctttgagaggatctggaggagttttgttgaataagcaaggaaagagatttgttaatgaattggatttgagatcggttgtttctaaggctattatggaacaaggagctgaatatcctggatctggaggatctatgtttgcttattgtgttttgaatgctgctgctcaaaagttgtttggagtttcttctcatgaattttattggaagaagatgggattgtttgttaaggctgatactatgagagatttggctgctttgattggatgtcctgttgaatctgttcaacaaactttggaagaatatgaaagattgtctatttctcaaagatcgtgtcctattactagaaagtctgtttatccttgtgttttgggaactaagggaccttattatgttgcttttgttactccttctattcattatactatgggaggatgtttgatttctccttctgctgaaattcaaatgaagaatacttcttctagggctcctttgtctcattctaatcctattttgggattgtttggagctggagaagttactggaggagttcatggaggaaatagattgggaggaaattctttgttggaatgtgttgtttttggaagaattgctggagatagagcttctactattttgcaaagaaagtcttctgctttgtcttttaaggtttggactactgttgttttgagagaagttagagaaggaggagtttatggagctggatctagggttttgagatttaatttgcctggagctttgcaaagatcgggattgtctttgggacaatttattgctattagaggagattgggatggacaacaattgattggatattattctcctattactttgcctgatgatttgggaatgattgatattttggctagatcggataagggaactttgagagaatggatttctgctttggaacctggagatgctgttgaaatgaaggcttgtggaggattggttattgaaagaagattgtctgataagcattttgtttttatgggacatattattaataagttgtgtttgattgctggaggaactggagttgctcctatgttgcaaattattaaggctgcttttatgaagccttttattgatactttggaatctgttcatttgatttatgctgctgaagatgttactgaattgacttatagagaagttttggaagaaagaagaagagaatctaggggaaagtttaagaagacttttgttttgaatagacctcctcctttgtggactgatggagttggatttattgatagaggaattttgactaatcatgttcaacctccttctgataatttgttggttgctatttgtggacctcctgttatgcaaagaattgttaaggctactttgaagactttgggatataatatgaatttggttagaactgttgatgaaactgaaccttctggatcttctaagatttaa

SEQ ID NO: 38 - protein, 1142 aa, Saccharomyces cerevisiae
MVDGRSSASIVAVDPERAARERDAAARALLQDSPLHTTMQYATSGLELTVPYALKVVASADTFDRAKEVADEVLRCAWQLADTVLNSFNPNSEVSLVGRLPVGQKHQMSAPLKRVMACCQRVYNSSAGCFDPSTAPVAKALREIALGKERNNACLEALTQACTLPNSFVIDFEAGTISRKHEHASLDLGGVSKGYIVDYVIDNINAAGFQNVFFDWGGDCRASGMNARNTPWVVGITRPPSLDMLPNPPKEASYISVISLDNEALATSGDYENLIYTADDKPLTCTYDWKGKELMKPSQSNIAQVSVKCYSAMYADALATACFIKRDPAKVRQLLDGWRYVRDTVRDYRVYVRENERVAKMFEIATEDAEMRKRRISNTLPARVIVVGGGLAGLSAAIEAAGCGAQVVLMEKEAKLGGNSAKATSGINGWGTRAQAKASIVDGGKYFERDTYKSGIGGNTDPALVKTLSMKSADAIGWLTSLGVPLTVLSQLGGHSRKRTHRAPDKKDGTPLPIGFTIMKTLEDHVRGNLSGRITIMENCSVTSLLSETKERPDGTKQIRVTGVEFTQAGSGKTTILADAVILATGGFSNDKTADSLLREHAPHLVNFPTTNGPWATGDGVKLAQRLGAQLVDMDKVQLHPTGLINPKDPANPTKFLGPEALRGSGGVLLNKQGKRFVNELDLRSVVSKAIMEQGAEYPGSGGSMFAYCVLNAAAQKLFGVSSHEFYWKKMGLFVKADTMRDLAALIGCPVESVQQTLEEYERLSISQRSCPITRKSVYPCVLGTKGPYYVAFVTPSIHYTMGGCLISPSAEIQMKNTSSRAPLSHSNPILGLFGAGEVTGGVHGGNRLGGNSLLECVVFGRIAGDRASTILQRKSSALSFKVWTTVVLREVREGGVYGAGSRVLRFNLPGALQRSGLSLGQFIAIRGDWDGQQLIGYYSPITLPDDLGMIDILARSDKGTLREWISALEPGDAVEMKACGGLVIERRLSDKHFVFMGHIINKLCLIAGGTGVAPMLQIIKAAFMKPFIDTLESVHLIYAAEDVTELTYREVLEERRRESRGKFKKTFVLNRPPPLWTDGVGFIDRGILTNHVQPPSDNLLVAICGPPVMQRIVKATLKTLGYNMNLVRTVDETEPSGSSKI

SEQ ID NO: 39 - DNA, 957 bp, Methylobacterium extorquens
  CDS (1)..(957)
atgtcttttagattgcaacctgctcctcctgctagacctaatagatgtcaattgtttggacctggatctaggccagctttgtttgaaaagatggctgcttctgctgctgatgttattaatttggatttggaagattctgttgctcctgatgataaggctcaagctagagctaatattattgaagctattaatggattggattggggaagaaagtatttgtctgttagaattaatggattggatactcctttttggtatagagatgttgttgatttgttggaacaagctggagatagattggatcaaattatgattcctaaggttggatgtgctgctgatgtttatgctgttgatgctttggttactgctattgaaagagctaagggaagaactaagcctttgtcttttgaagttattattgaatctgctgctggaattgctcatgttgaagaaattgctgcttcttctcctagattgcaagctatgtctttgggagctgctgattttgctgcttctatgggaatgcaaactactggaattggaggaactcaagaaaattattatatgttgcatgatggacaaaagcattggtctgatccttggcattgggctcaagctgctattgttgctgcttgtagaactcatggaattttgcctgttgatggaccttttggagatttttctgatgatgaaggatttagagctcaagctagaagatcggctactttgggaatggttggaaagtgggctattcatcctaagcaagttgctttggctaatgaagtttttactccttctgaaactgctgttactgaagctagagaaattttggctgctatggatgctgctaaggctagaggagaaggagctactgtttataagggaagattggttgatattgcttctattaagcaagctgaagttattgttagacaagctgaaatgatttctgcttaa

SEQ ID NO: 40 - protein, 318 aa, Methylobacterium extorquens; identical to SEQ ID NO: 8

SEQ ID NO: 41 - DNA, 1581 bp, Ralstonia eutropha
  CDS (1)..(1581)
atggctcaatatcaagatgatattaaggctgttgctggattgaaggaaaatcatggatctgcttggaatgctattaatcctgaatatgctgctagaatgagagctcaaaataagtttaagactggattggatattgctaagtacactgctaagattatgagagctgatatggctgcttatgatgctgattcttctaagtacactcaatctttgggatgttggcatggatttattggacaacaaaagatgatttctattaagaagcattttaattctactgaaagaagatatttgtatttgtctggatggatggttgctgctttgagatcggaatttggacctttgcctgatcaatctatgcatgaaaagacttctgtttctgctttgattagagaattgtacacttttttgagacaagctgatgctagagaattgggaggattgtttagagaattggatgctgctcaaggacctgctaaggctgctattcaagctaagattgataatcatgttactcatgttgttcctattattgctgatattgatgctggatttggaaatgctgaagctacttatttgttggctaagcaatttattgaagctggagcttgttgtattcaaattgaaaatcaagtttctgatgaaaagcaatgtggacatcaagatggaaaggttactgttcctcatgaagattttttggctaagattagagctattagatatgcttttttggaattgggagttgatgatggaattattgttgctagaactgattctttgggagctggattgactaagcaaattgctgttactaatactcctggagatttgggagatcaatataattcttttttggattgtgaagaattgtctgctgatcaattgggaaatggagatgttattattaagagagatggaaagttgttgagacctaagagattgccttctaatttgtttcaatttagagctggaactggagaagctagatgtgttttggattgtgttactgctttgcaaaatggagctgatttgttgtggattgaaactgaaaagcctcatattgctcaaattggaggaatggtttctgaaattagaaaggttattcctaatgctaagttggtttataataattctccttcttttaattggactttgaattttagacaacaagcttatgatgctatgaaggctgctggaaaggatgtttctgcttatgatagagctcaattgatgtctgttgaatatgatcaaactgaattggctaagttggctgatgaaaagattagaacttttcaagctgatgcttctagggaagctggaatttttcatcatttgattactttgcctacttatcatactgctgctttgtctactgataatttggctaaggaatattttggagatcaaggaatgttgggatatgttgctggagttcaaagaaaggaaattagacaaggaattgcttgtgttaagcatcaaaatatgtctggatctgatattggagatgatcataaggaatatttttctggagaagctgctttgaaggctgctggaaaggataatactatgaatcaattttaa

SEQ ID NO: 42 - protein, 526 aa, Ralstonia eutropha
MAQYQDDIKAVAGLKENHGSAWNAINPEYAARMRAQNKFKTGLDIAKYTAKIMRADMAAYDADSSKYTQSLGCWHGFIGQQKMISIKKHFNSTERRYLYLSGWMVAALRSEFGPLPDQSMHEKTSVSALIRELYTFLRQADARELGGLFRELDAAQGPAKAAIQAKIDNHVTHVVPIIADIDAGFGNAEATYLLAKQFIEAGACCIQIENQVSDEKQCGHQDGKVTVPHEDFLAKIRAIRYAFLELGVDDGIIVARTDSLGAGLTKQIAVTNTPGDLGDQYNSFLDCEELSADQLGNGDVIIKRDGKLLRPKRLPSNLFQFRAGTGEARCVLDCVTALQNGADLLWIETEKPHIAQIGGMVSEIRKVIPNAKLVYNNSPSFNWTLNFRQQAYDAMKAAGKDVSAYDRAQLMSVEYDQTELAKLADEKIRTFQADASREAGIFHHLITLPTYHTAALSTDNLAKEYFGDQGMLGYVAGVQRKEIRQGIACVKHQNMSGSDIGDDHKEYFSGEAALKAAGKDNTMNQF

SEQ ID NO: 43 - DNA, 3306 bp, Homo sapiens
  CDS (1)..(3306)
atgtctgctaaggctatttctgaacaaactggaaaggaattgttgtataagtttatttgtactacttctgctattcaaaatagatttaagtatgctagagttactcctgatactgattgggctagattgttgcaagatcatccttggttgttgtctcaaaatttggttgttaagcctgatcaattgattaagagaagaggaaagttgggattggttggagttaatttgactttggatggagttaagtcttggttgaagcctagattgggacaagaagctactgttggaaaggctactggatttttgaagaattttttgattgaaccttttgttcctcattctcaagctgaagaattttatgtttgtatttatgctactagagaaggagattatgttttgtttcatcatgaaggaggagttgatgttggagatgttgatgctaaggctcaaaagttgttggttggagttgatgaaaagttgaatcctgaagatattaagaagcatttgttggttcatgctcctgaagataagaaggaaattttggcttcttttatttctggattgtttaatttttatgaagatttgtattttacttatttggaaattaatcctttggttgttactaaggatggagtttatgttttggatttggctgctaaggttgatgctactgctgattatatttgtaaggttaagtggggagatattgaatttcctcctccttttggaagagaagcttatcctgaagaagcttatattgctgatttggatgctaagtctggagcttctttgaagttgactttgttgaatcctaagggaagaatttggactatggttgctggaggaggagcttctgttgtttattctgatactatttgtgatttgggaggagttaatgaattggctaattatggagaatattctggagctccttctgaacaacaaacttatgattatgctaagactattttgtctttgatgactagagaaaagcatcctgatggaaagattttgattattggaggatctattgctaattttactaatgttgctgctacttttaagggaattgttagagctattagagattatcaaggacctttgaaggaacatgaagttactatttttgttagaagaggaggacctaattatcaagaaggattgagagttatgggagaagttggaaagactactggaatacctattcatgtttttggaactgaaactcatatgactgctattgttggaatggctttgggacatagacctattcctaatcaacctcctactgctgctcatactgctaattttttgttgaatgcttctggatctacttctactcctgctccttctaggactgcttctttttctgaatctagggctgatgaagttgctcctgctaagaaggctaagcctgctatgcctcaagattctgttccttctcctagatcgttgcaaggaaagtctactactttgttttctaggcatactaaggctattgtttggggaatgcaaactagagctgttcaaggaatgttggattttgattatgtttgttctagggatgaaccttctgttgctgctatggtttatccttttactggagatcataagcaaaagttttattggggacataaggaaattttgattcctgtttttaagaatatggctgatgctatgagaaagcatcctgaagttgatgttttgattaattttgcttctttgagatcggcttatgattctactatggaaactatgaattatgctcaaattagaactattgctattattgctgaaggaatacctgaagctttgactagaaagttgattaagaaggctgatcaaaagggagttactattattggacctgctactgttggaggaattaagcctggatgttttaagattggaaatactggaggaatgttggataatattttggcttctaagttgtatagacctggatctgttgcttatgtttctaggtcgggaggaatgtctaatgaattgaataatattatttctaggactactgatggagtttatgaaggagttgctattggaggagatagatatcctggatctacttttatggatcatgttttgagatatcaagatactcctggagttaagatgattgttgttttgggagaaattggaggaactgaagaatataagatttgtagaggaattaaggaaggaagattgactaagcctattgtttgttggtgtattggaacttgtgctactatgttttcttctgaagttcaatttggacatgctggagcttgtgctaatcaagcttctgaaactgctgttgctaagaatcaagctttgaaggaagctggagtttttgttcctagatcgtttgatgaattgggagaaattattcaatctgtttatgaagatttggttgctaatggagttattgttcctgctcaagaagttcctcctcctactgttcctatggattattcttgggctagagaattgggattgattagaaagcctgcttcttttatgacttctatttgtgatgaaagaggacaagaattgatttatgctggaatgcctattactgaagtttttaaggaagaaatgggaattggaggagttttgggattgttgtggtttcaaaagagattgcctaagtattcttgtcaatttattgaaatgtgtttgatggttactgctgatcatggacctgctgtttctggagctcataatactattatttgtgctagagctggaaaggatttggtttcttctttgacttctggattgttgactattggagatagatttggaggagctttggatgctgctgctaagatgttttctaaggcttttgattctggaattattcctatggaatttgttaataagatgaagaaggaaggaaagttgattatgggaattggacatagagttaagtctattaataatcctgatatgagagttcaaattttgaaggattatgttagacaacattttcctgctactcctttgttggattatgctttggaagttgaaaagattactacttctaagaagcctaatttgattttgaatgttgatggattgattggagttgcttttgttgatatgttgagaaattgtggatcttttactagagaagaagctgatgaatatattgatattggagctttgaatggaatttttgttttgggaagatcgatgggatttattggacattatttggatcaaaagagattgaagcaaggattgtatagacatccttgggatgatatttcttatgttttgcctgaacatatgtctatgtaa

SEQ ID NO: 44 - protein, 1101 aa, Homo sapiens
MSAKAISEQTGKELLYKFICTTSAIQNRFKYARVTPDTDWARLLQDHPWLLSQNLVVKPDQLIKRRGKLGLVGVNLTLDGVKSWLKPRLGQEATVGKATGFLKNFLIEPFVPHSQAEEFYVCIYATREGDYVLFHHEGGVDVGDVDAKAQKLLVGVDEKLNPEDIKKHLLVHAPEDKKEILASFISGLFNFYEDLYFTYLEINPLVVTKDGVYVLDLAAKVDATADYICKVKWGDIEFPPPFGREAYPEEAYIADLDAKSGASLKLTLLNPKGRIWTMVAGGGASVVYSDTICDLGGVNELANYGEYSGAPSEQQTYDYAKTILSLMTREKHPDGKILIIGGSIANFTNVAATFKGIVRAIRDYQGPLKEHEVTIFVRRGGPNYQEGLRVMGEVGKTTGIPIHVFGTETHMTAIVGMALGHRPIPNQPPTAAHTANFLLNASGSTSTPAPSRTASFSESRADEVAPAKKAKPAMPQDSVPSPRSLQGKSTTLFSRHTKAIVWGMQTRAVQGMLDFDYVCSRDEPSVAAMVYPFTGDHKQKFYWGHKEILIPVFKNMADAMRKHPEVDVLINFASLRSAYDSTMETMNYAQIRTIAIIAEGIPEALTRKLIKKADQKGVTIIGPATVGGIKPGCFKIGNTGGMLDNILASKLYRPGSVAYVSRSGGMSNELNNIISRTTDGVYEGVAIGGDRYPGSTFMDHVLRYQDTPGVKMIVVLGEIGGTEEYKICRGIKEGRLTKPIVCWCIGTCATMFSSEVQFGHAGACANQASETAVAKNQALKEAGVFVPRSFDELGEIIQSVYEDLVANGVIVPAQEVPPPTVPMDYSWARELGLIRKPASFMTSICDERGQELIYAGMPITEVFKEEMGIGGVLGLLWFQKRLPKYSCQFIEMCLMVTADHGPAVSGAHNTIICARAGKDLVSSLTSGLLTIGDRFGGALDAAAKMFSKAFDSGIIPMEFVNKMKKEGKLIMGIGHRVKSINNPDMRVQILKDYVRQHFPATPLLDYALEVEKITTSKKPNLILNVDGLIGVAFVDMLRNCGSFTREEADEYIDIGALNGIFVLGRSMGFIGHYLDQKRLKQGLYRHPWDDISYVLPEHMSM

SEQ ID NO: 45 - DNA, 3600 bp, Synechocystis PCC6803
  CDS (1)..(3600)
atgagtttacctacctatgccaccctcgacggtaatgaagcggtggcccgtgtggcctacctgctcagtgaagtgattgccatttatcccatcaccccttcctcgcccatgggggaatggtccgatgcttgggcagcagaacaccggcccaatttgtggggcaccgtaccattggtggtggaaatgcaaagcgaggggggagccgccggtactgtccatggcgctctgcaatcgggagctttgaccacaacatttaccgcttcccagggcttaatgttgatgttgcccaatatgcacaaaattgctggggaattaacagccatggttttgcatgtggcggcccgttctttagcggcccagggcctatctatttttggggatcacagtgatgtgatggcggccagaaatacgggctttgccatgttaagttccaattctgtccaggaagcccacgattttgccctcattgccacggccaccagctttgccaccaggataccgggactgcacttttttgatggttttcgcacttcccacgaagaacaaaaaattgagcttttaccccaggaagtactccgtggtttgattaaggatgaggatgtgctagcccaccggggacgggctttgacccccgatcgcccgaagttgcgggggacggcccaaaatccggatgtctatttccaagctagggaaacggttaatcccttttatgccagttatcccaacgtgctggagcaggtgatggaacaatttggccagctaaccggccgccattaccgtccctatgaatattgtggccatccggaagcggaacgggtgattgtgctgatgggttctggtgcggaaacggcccaggaaacggtggattttctaactgcccaaggggaaaaggttggtttactgaaagtacgcctctatcggccctttgctggcgatcgcctggttaatgctctaccaaaaacggtgcaaaaaatagcggtgctggaccggtgtaaggaaccggggagcattggggaacccctctatcaggatgtgctgacggccttttttgaagcgggcatgatgccgaaaattattggtggccgttacggtctgtcatccaaggaatttacccccgccatggttaaaggggtgttggaccatttaaatcaaaccaaccccaaaaaccatttcaccgtaggcattaacgatgatttgagccacaccagcatcgactatgaccccagtttttccacggaagcagattctgtcgtccgggcaattttctacggtctcggttccgacggtacggtgggggccaataagaactccatcaaaatcattggcgaagatacggataactacgcccagggttattttgtttacgactcgaaaaaatccggttctgtaaccgtttcccatctgcgctttggccctaatcccatcctgtccacttacctgattagccaagccaattttgtcgcctgtcaccagtgggaatttttggaacagtttgaagtcttggaaccagccgttgatggcggcgttttcctggtcaatagcccctacggcccagaggaaatttggcgagagtttccccgcaaagtacaacaggaaattattgacaaaaatctcaaggtttacaccatcaatgccaatgacgtagccagggatgcgggcatgggccgccgcaccaacacagtcatgcaaacctgtttctttgccctagcgggagtgttaccccgggaagaggcgatcgccaaaattaagcagtcggtccaaaaaacctacggcaaaaagggtcaggaaattgtcgagatgaatattaaagcggtggattccaccctggcccatctctatgaagtgtccgtaccggaaacggtgagcgacgatgcccctgctatgcggccggtggtgcctgataacgccccggtgtttgtgcgggaagtgttaggaaaaatcatggcccggcaaggggatgatctcccggtcagtgctttaccctgcgatggcacctatcccaccgccactacccaatgggaaaaacgcaacgtgggccacgaaattcccgtttgggaccccgatgtttgtgtgcaatgcggcaaatgcgtcattgtttgtccccatgctgtgattcggggcaaagtttacgaggaggcagaattggccaatgctccggtcagtttcaaatttaccaatgccaaagaccatgattggcaaggttctaagttcaccatccaggtagccccggaagattgcaccggttgcggcatctgtgtggacgtatgcccggctaaaaataaatcccagcctcgtttaagggcgattaatatggctccccagttacccttgcgggaacaggaacgggagaattgggactttttcctagatttgcccaaccccgatcgcctcagtttgaatttgaacaaaatcagccatcaacagatgcaggagccgttatttgaattttctggagcctgtgccggttgtggggaaaccccttatttgaaactggtcagtcaattatttggcgatcgcatgttagtggccaacgccaccggttgctcttccatctatggcggcaacttaccgacaactccctgggcccaaaatgctgagggtcgcggtcccgcttggtccaattccctgtttgaagataacgctgaatttggccttggtttccgagtggcgatcgacaagcaaacggaatttgcaggggaattgctaaaaacctttgctggggagttgggagacagtttggtaagtgaaattctcaacaatgcccaaaccactgaagcggatatttttgaacaacggcaattggtagaacaggttaagcaacgtttgcaaaatctggaaactccccaagcccaaatgttcctttctgtagcggattacctcgtgaagaaaagcgtttggattattggtggcgatggctgggcctacgacattgggtacggcggtttggatcacgtcctcgccagtgggcgtaatgtcaatatcttggtgatggatacggaagtctattccaacaccgggggccaagcctccaaagccactccccgggccgctgtagctaaattcgccgctgggggtaaaccctctcccaaaaaagatttgggcttaatggccatgacctacggcaacgtctatgtggccagtatcgccatgggagccaaaaatgagcagtccattaaagcctttatggaagcggaagcctatcccggtgtctcgttaattattgcctactcccactgcattgcccacggcattaatatgaccaccgcgatgaaccatcaaaaagagttggtggacagcggtcgttggttgctctaccgctataaccctttgttggcggatgaaggtaaaaatcccctgcaattggatatgggatcgccaaaagtagccattgacaaaacggtctattcggaaaatcgctttgccatgctcacccgcagtcaaccagaggaggccaaacgcttaatgaagttagctcaaggggatgtgaacactcgctgggccatgtacgaatatctggcgaaacgttctctgggtggggaaattaacggtaacaaccatggtgtttccccatctccggaggtaattgctaaatctgtttag

SEQ ID NO: 46 - protein, 1199 aa, Synechocystis PCC6803
MSLPTYATLDGNEAVARVAYLLSEVIAIYPITPSSPMGEWSDAWAAEHRPNLWGTVPLVVEMQSEGGAAGTVHGALQSGALTTTFTASQGLMLMLPNMHKIAGELTAMVLHVAARSLAAQGLSIFGDHSDVMAARNTGFAMLSSNSVQEAHDFALIATATSFATRIPGLHFFDGFRTSHEEQKIELLPQEVLRGLIKDEDVLAHRGRALTPDRPKLRGTAQNPDVYFQARETVNPFYASYPNVLEQVMEQFGQLTGRHYRPYEYCGHPEAERVIVLMGSGAETAQETVDFLTAQGEKVGLLKVRLYRPFAGDRLVNALPKTVQKIAVLDRCKEPGSIGEPLYQDVLTAFFEAGMMPKIIGGRYGLSSKEFTPAMVKGVLDHLNQTNPKNHFTVGINDDLSHTSIDYDPSFSTEADSVVRAIFYGLGSDGTVGANKNSIKIIGEDTDNYAQGYFVYDSKKSGSVTVSHLRFGPNPILSTYLISQANFVACHQWEFLEQFEVLEPAVDGGVFLVNSPYGPEEIWREFPRKVQQEIIDKNLKVYTINANDVARDAGMGRRTNTVMQTCFFALAGVLPREEAIAKIKQSVQKTYGKKGQEIVEMNIKAVDSTLAHLYEVSVPETVSDDAPAMRPVVPDNAPVFVREVLGKIMARQGDDLPVSALPCDGTYPTATTQWEKRNVGHEIPVWDPDVCVQCGKCVIVCPHAVIRGKVYEEAELANAPVSFKFTNAKDHDWQGSKFTIQVAPEDCTGCGICVDVCPAKNKSQPRLRAINMAPQLPLREQERENWDFFLDLPNPDRLSLNLNKISHQQMQEPLFEFSGACAGCGETPYLKLVSQLFGDRMLVANATGCSSIYGGNLPTTPWAQNAEGRGPAWSNSLFEDNAEFGLGFRVAIDKQTEFAGELLKTFAGELGDSLVSEILNNAQTTEADIFEQRQLVEQVKQRLQNLETPQAQMFLSVADYLVKKSVWIIGGDGWAYDIGYGGLDHVLASGRNVNILVMDTEVYSNTGGQASKATPRAAVAKFAAGGKPSPKKDLGLMAMTYGNVYVASIAMGAKNEQSIKAFMEAEAYPGVSLIIAYSHCIAHGINMTTAMNHQKELVDSGRWLLYRYNPLLADEGKNPLQLDMGSPKVAIDKTVYSENRFAMLTRSQPEEAKRLMKLAQGDVNTRWAMYEYLAKRSLGGEINGNNHGVSPSPEVIAKSV

SEQ ID NO: 47 - DNA, 3411 bp, Lactococcus lactis
  CDS (1)..(3411)
atgaagaagttgttggttgctaatagaggagaaattgctgttagagtttttagagcttgtaatgaattgggattgtctactgttgctgtttatgctagagaagatgaatattctgttcatagatttaaggctgatgaatcttatttgattggacaaggaaagaagcctattgatgcttatttggatattgatgatattattagagttgctttggaatctggagctgatgctattcatcctggatatggattgttgtctgaaaatttggaatttgctactaaggttagagctgctggattggtttttgttggacctgaattgcatcatttggatatttttggagataagattaaggctaaggctgctgctgatgaagctaaggttcctggaatacctggaactaatggagctgttgatattgatggagctttggaatttgctaagacttatggatatcctgttatgattaaggctgctttgggaggaggaggaagaggaatgagagttgcttctaatgatgctgaaatgcatgatggatatgctagagctaagtctgaagctattggagcttttggatctggagaaatttatgttgaaaagtatattgaaaatcctaagcatattgaagttcaaattttgggagattctcatggaaatattattcatttgcatgaaagagattgttctgttcaaagaagaaatcaaaaggttattgaaattgctcctgctgttggattgtctttggattttagaaatgaaatttgtgaagctgctgttaagttgtgtaagaatgttggatatgttaatgctggaactgttgaatttttggttaaggatgataagttttattttattgaagttaatcctagagttcaagttgaacatactattactgaattgattactggagttgatattgttcaagctcaaattttgattgctcaaggaaaggatttgcatagagaaattggattgcctgctcaatctgaaattcctttgttgggatctgctattcaatgtagaattactactgaagatcctcaaaatggatttttgcctgatactggaaagattgatacttatagatcgcctggaggatttggagttagattggatgttggaaatgcttatgctggatatgaagttactccttattttgattctttgttggttaaggtttgtacttttgctaatgaattttctgatactgttagaaagatggatagagttttgcatgaatttagaattagaggagttaagactaatattccttttttgattaatgttattgctaatgaaaattttacttctggacaagctactactacttttattgataatactccttctttgtttaattttcctcatttgagagatagaggaactaagactttgcattatttgtctatgattactgttaatggatttcctggaattgaaaatactgaaaagagacattttgaagaacctagacaacctttgttgaatttggaaaagaagaagactgctaagaatattttggatgaacaaggagctgatgctgttgttgattatgttaagaatactaaggaagttttgttgactgatactactttgagagatgctcatcaatctttgttggctactagattgagattgcaagatatgaagggaattgctcaagctattgatcaaggattgcctgaattgttttctgctgaaatgtggggaggagctacttttgatgttgcttatagatttttgaatgaatctccttggtatagattgagaaagttgagaaagttgatgcctaatactatgtttcaaatgttgtttagaggatctaatgctgttggatatcaaaattatcctgataatgttattgaagaatttattagagttgctgctcatgaaggaattgatgtttttagaatttttgattctttgaattggttgcctcaaatggaaaagtctattcaagctgttagagataatggaaagattgctgaagctactatttgttatactggagatattttggatccttctaggccaaagtataatattcaatattataaggatttggctaaggaattggaagctactggagctcatattttggctgttaaggatatggctggattgttgaagcctcaagctgcttatagattgatttctgaattgaaggatactgttgatttgcctattcatttgcatactcatgatacttctggaaatggaattattacttattctggagctactcaagctggagttgatattattgatgttgctactgcttctttggctggaggaacttctcaaccttctatgcaatctatttattatgctttggaacatggacctagacatgcttctattaatgttaagaatgctgaacaaattgatcattattgggaagatgttagaaagtattatgctccttttgaagctggaattacttctcctcaaactgaagtttatatgcatgaaatgcctggaggacaatatactaatttgaagtctcaagctgctgctgttggattgggacatagatttgatgaaattaagcaaatgtatagaaaggttaatatgatgtttggagatattattaaggttactccttcttctaaggttgttggagatatggctttgtttatgattcaaaatgatttgactgaagaagatgtttatgctagaggaaatgaattgaattttcctgaatctgttgtttctttttttagaggagatttgggacaacctgttggcggatttcctgaagaattgcaaaagattattgttaaggataaggctgttattactgatagacctggattgcatgctgaaaaggttgattttgaaactgttaaggctgatttggaacaaaagattggatatgaacctggagatcatgaagttatttcttatattatgtatcctcaagtttttttggattatcaaaagatgcaaagagaatttggagctgttactttgttggatactcctacttttttgcatggaatgagattgaatgaaaagattgaagttcaaattgaaaagggaaagactttgtctattagattggatgaaattggagaacctgatttggctggaaatagagttttgttttttaatttgaatggacaaagaagagaagttgttattaatgatcaatctgttcaaactcaagttgttgctaagagaaaggctgaaactggaaatcctaatcaaattggagctactatgcctggatctgttttggaaattttggttaaggctggagataaggttcaaaagggacaagctttgatggttactgaagctatgaagatggaaactactattgaagctccttttgatggagaaattgttgatttgcatgttgttaagggagaagctattcaaactcaagatttgttgattgaaattaat

SEQ ID NO: 48 - protein, 1137 aa, Lactococcus lactis
MKKLLVANRGEIAVRVFRACNELGLSTVAVYAREDEYSVHRFKADESYLIGQGKKPIDAYLDIDDIIRVALESGADAIHPGYGLLSENLEFATKVRAAGLVFVGPELHHLDIFGDKIKAKAAADEAKVPGIPGTNGAVDIDGALEFAKTYGYPVMIKAALGGGGRGMRVASNDAEMHDGYARAKSEAIGAFGSGEIYVEKYIENPKHIEVQILGDSHGNIIHLHERDCSVQRRNQKVIEIAPAVGLSLDFRNEICEAAVKLCKNVGYVNAGTVEFLVKDDKFYFIEVNPRVQVEHTITELITGVDIVQAQILIAQGKDLHREIGLPAQSEIPLLGSAIQCRITTEDPQNGFLPDTGKIDTYRSPGGFGVRLDVGNAYAGYEVTPYFDSLLVKVCTFANEFSDTVRKMDRVLHEFRIRGVKTNIPFLINVIANENFTSGQATTTFIDNTPSLFNFPHLRDRGTKTLHYLSMITVNGFPGIENTEKRHFEEPRQPLLNLEKKKTAKNILDEQGADAVVDYVKNTKEVLLTDTTLRDAHQSLLATRLRLQDMKGIAQAIDQGLPELFSAEMWGGATFDVAYRFLNESPWYRLRKLRKLMPNTMFQMLFRGSNAVGYQNYPDNVIEEFIRVAAHEGIDVFRIFDSLNWLPQMEKSIQAVRDNGKIAEATICYTGDILDPSRPKYNIQYYKDLAKELEATGAHILAVKDMAGLLKPQAAYRLISELKDTVDLPIHLHTHDTSGNGIITYSGATQAGVDIIDVATASLAGGTSQPSMQSIYYALEHGPRHASINVKNAEQIDHYWEDVRKYYAPFEAGITSPQTEVYMHEMPGGQYTNLKSQAAAVGLGHRFDEIKQMYRKVNMMFGDIIKVTPSSKVVGDMALFMIQNDLTEEDVYARGNELNFPESVVSFFRGDLGQPVGGFPEELQKIIVKDKAVITDRPGLHAEKVDFETVKADLEQKIGYEPGDHEVISYIMYPQVFLDYQKMQREFGAVTLLDTPTFLHGMRLNEKIEVQIEKGKTLSIRLDEIGEPDLAGNRVLFFNLNGQRREVVINDQSVQTQVVAKRKAETGNPNQIGATMPGSVLEILVKAGDKVQKGQALMVTEAMKMETTIEAPFDGEIVDLHVVKGEAIQTQDLLIEIN

SEQ ID NO: 49 - DNA, 1173 bp, Methylobacterium extorquens
  CDS (1)..(1173)
atggacgttcacgagtaccaagccaaggagctgctcgcgagcttcggggtcgccgtcccgaagggcgccgtggctttcagcccggatcaagcggtctatgcggcgaccgagctcggcggctcgttctgggcggtgaaggctcagatccatgccggcgcgcgcggcaaggcgggcgggatcaagctttgccgcacctacaatgaagtgcgcgacgccgcccgcgacctgctgggaaaacgcctcgtgacgctccagaccggccccgagggcaagccggtgcagcgcgtctacgtcgagaccgccgacccgttcgagcgtgaactctatctcggctacgtgctcgatcggaaggccgagcgcgtccgtgtcatcgcctcccagcgcggcggcatggatatcgaggagatcgccgccaaggagcccgaggcgctgatccaggtcgtggtcgagccggcggtgggcctgcagcagttccaggcccgcgagatcgcgttccagctcggcctcaacatcaagcaggtctcggccgcggtgaagaccatcatgaacgcctaccgggcgttccgcgactgcgacggcaccatgctggagatcaacccgctcgtcgtcaccaaggacgaccgggttctggcactcgacgccaagatgtccttcgacgacaacgccctgttccgccgccgcaacatcgcggacatgcacgatccatcgcagggcgatccccgcgaggcccaggctgccgagcacaatctcagctatatcggcctcgagggcgaaattggctgcatcgtcaacggcgcgggtctggccatggcgaccatggacatgatcaagcacgcgggcggcgagccggcaaacttcctggatgtgggcggcggtgccagcccggaccgcgtcgccacggccttccgcctcgttctgtcggaccgcaacgtgaaggcgatcctcgtcaacatcttcgccggcatcaaccgctgcgactgggtcgcggagggcgtggtcaaggccgcgcgcgaggtgaagatcgacgtgccgctcatcgtgcggctcgccggcacgaacgtcgatgaaggcaagaagatcctcgccgagagcgggctcgacctcatcaccgccgacacccttacggaagccgcgcgcaaggctgtcgaagcctgccacggcgccaagcactga

SEQ ID NO: 50 - protein, 390 aa, Methylobacterium extorquens
MDVHEYQAKELLASFGVAVPKGAVAFSPDQAVYAATELGGSFWAVKAQIHAGARGKAGGIKLCRTYNEVRDAARDLLGKRLVTLQTGPEGKPVQRVYVETADPFERELYLGYVLDRKAERVRVIASQRGGMDIEEIAAKEPEALIQVVVEPAVGLQQFQAREIAFQLGLNIKQVSAAVKTIMNAYRAFRDCDGTMLEINPLVVTKDDRVLALDAKMSFDDNALFRRRNIADMHDPSQGDPREAQAAEHNLSYIGLEGEIGCIVNGAGLAMATMDMIKHAGGEPANFLDVGGGASPDRVATAFRLVLSDRNVKAILVNIFAGINRCDWVAEGVVKAAREVKIDVPLIVRLAGTNVDEGKKILAESGLDLITADTLTEAARKAVEACHGAKH

SEQ ID NO: 51 - DNA, 1200 bp, Ruegeria pomeroyi; identical to SEQ ID NO: 27
  CDS (1)..(1200)

SEQ ID NO: 52 - protein, 399 aa, Ruegeria pomeroyi; identical to SEQ ID NO: 28

SEQ ID NO: 53 - DNA, 1167 bp, Ralstonia eutropha
  CDS (1)..(1167)
atgaatatccatgagtaccaaggcaaggaaatcctgcgcaaatacaatgtgccggttccgcgcggcattccggccttctcggtcgacgaggccatcaaggctgctgaaaccctgggcggcccggtgtgggtcgtgaaggcacagattcatgcgggtggccgtggcaagggcggcggcgtgaaggttgccaagagcatcgagcaggtcaaggaatacgccagcagcatcctgggcatgacgctggtgacgcaccagaccggtccggaaggcaagctggtcaagcgcctgctgattgaagaaggcgcggacatcaagaaggaactgtacgtgtcgctggtggtggaccgtgtgtcgcaacaagtggcgctgatggcctcgagcgaaggcggcatggacatcgaagaagtcgccgaatcgcacccggaaaagatccacacgctgctgatcgatccgcaagccggtctgcaagacgctcaggctgacgacatcgctcgcaagatcggcgtgccggatgcttcgatcgcgcaagcccgccaagctctgcaaggcctgtacaaggcgttctgggaaaccgacgcttcgcaagctgaaatcaacccgctgatcctgaccggcgacggcaaggtcatcgcactggacgccaagttcaacttcgactcgaacgcgctgttccgtcacccggaaatcgtggcgtaccgcgatctggatgaagaagacccggcggaaatcgaagcctcgaagttcgacctggcttacatctcgctcgacggcaacatcggctgcctggtgaatggcgctggtctggccatggcgacgatggacaccatcaagctgttcggcggcgagccggccaacttcctcgacgtgggcggcggtgccaccaccgagaaggtgaccgaagccttcaagctgatgctgaagaacccggacgtgaaggccattctggtcaacatcttcggcggcatcatgcgttgcgacgtgatcgccgaaggcgtgatcgctgcagccaaggctgtgtcgctgtcggtgccgctggtggtgcgcatgaagggtaccaacgaagacctcggcaagaagatgctggccgactcgggtctgcccatcatcgccgcagacacgatggcagaggccgccgagaaagtcgtggccgcagccgccggcaagtaa

SEQ ID NO: 54 - protein, 388 aa, Ralstonia eutropha
MNIHEYQGKEILRKYNVPVPRGIPAFSVDEAIKAAETLGGPVWVVKAQIHAGGRGKGGGVKVAKSIEQVKEYASSILGMTLVTHQTGPEGKLVKRLLIEEGADIKKELYVSLVVDRVSQQVALMASSEGGMDIEEVAESHPEKIHTLLIDPQAGLQDAQADDIARKIGVPDASIAQARQALQGLYKAFWETDASQAEINPLILTGDGKVIALDAKFNFDSNALFRHPEIVAYRDLDEEDPAEIEASKFDLAYISLDGNIGCLVNGAGLAMATMDTIKLFGGEPANFLDVGGGATTEKVTEAFKLMLKNPDVKAILVNIFGGIMRCDVIAEGVIAAAKAVSLSVPLVVRMKGTNEDLGKKMLADSGLPIIAADTMAEAAEKVVAAAAGK

SEQ ID NO: 55 - protein, 399 aa, Caulobacter vibrioides
MNIHEHQAKAVLAEFGAPVPRGFAAFTPDEAAAAAEKLGGPVFVVKSQIHAGGRGKGKFEGLGPDAKGGVRVVKSVEEVRSNAEEMLGRVLVTHQTGPKGKQVNRLYIEEGAAIAKEFYLSLLVDRASSKVSVVASTEGGMDIEDVAHSTPEKIHTFTIDPATGVWPTHHRALAKALGLTGGLAKEAASLLNQLYTAFMAKDMAMLEINPLIVTADDHLRVLDAKLSFDGNSLFRHPDIKALRDESEEDPKEIEASKYDLAYIALDGEIGCMVNGAGLAMATMDIIKLYGAEPANFLDVGGGASKEKVTAAFKIITADPAVKGILVNIFGGIMRCDIIAEGVIAAVKEVGLQVPLVVRLEGTNVELGKKIISESGLNVIAANDLSDGAEKIVAAVKGAR

SEQ ID NO: 56 - DNA, 1167 bp, Escherichia coli
  CDS (1)..(1167)
atgaatttgcatgaatatcaagctaagcaattgtttgctagatatggattgcctgctcctgttggatatgcttgtactactcctagagaagctgaagaagctgcttctaagattggagctggaccttgggttgttaagtgtcaagttcatgctggaggaagaggaaaggctggaggagttaaggttgttaattctaaggaagatattagagcttttgctgaaaattggttgggaaagagattggttacttatcaaactgatgctaatggacaacctgttaatcaaattttggttgaagctgctactgatattgctaaggaattgtatttgggagctgttgttgatagatcgtctaggagagttgtttttatggcttctactgaaggaggagttgaaattgaaaaggttgctgaagaaactcctcatttgattcataaggttgctttggatcctttgactggacctatgccttatcaaggaagagaattggcttttaagttgggattggaaggaaagttggttcaacaatttactaagatttttatgggattggctactatttttttggaaagagatttggctttgattgaaattaatcctttggttattactaagcaaggagatttgatttgtttggatggaaagttgggagctgatggaaatgctttgtttagacaacctgatttgagagaaatgagagatcaatctcaagaagatcctagagaagctcaagctgctcaatgggaattgaattatgttgctttggatggaaatattggatgtatggttaatggagctggattggctatgggaactatggatattgttaagttgcatggaggagaacctgctaattttttggatgttggaggaggagctactaaggaaagagttactgaagcttttaagattattttgtctgatgataaggttaaggctgttttggttaatatttttggaggaattgttagatgtgatttgattgctgatggaattattggagctgttgctgaagttggagttaatgttcctgttgttgttagattggaaggaaataatgctgaattgggagctaagaagttggctgattctggattgaatattattgctgctaagggattgactgatgctgctcaacaagttgttgctgctgttgaaggaaagtaa

SEQ ID NO: 57 - protein, 388 aa, Escherichia coli
MNLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLTDAAQQVVAAVEGK

SEQ ID NO: 58 - DNA, 891 bp, Methylobacterium extorquens
  CDS (1)..(891)
atgagcattctcatcgacgagaagaccccgatcctggttcagggcatcacgggcgacaagggcaccttccacgccaaggaaatgatcgcctacggctccaacgtcgtcggcggcgtcaccccgggcaagggcggcaagacccattgcggcgtgccggtgttcaacaccgtcaaagaggccgtggaggcgaccggcgccaccacctcgatcactttcgtggcgccccccttcgcggcggacgcgatcatggaggcggccgatgccggcctcaagctcgtctgctcgatcaccgacggcatccccgctcaggacatgatgcgggtgaaacgctacctccggcgctatccgaaggagaagcgcacgatggtggtgggcccgaactgcgcgggcatcatctcgcccggcaagtcgatgctcggcatcatgcccggccatatctacctgcccggcaaggtcggcgtcatctcccgctccggaaccctcggctacgaggccgccgcgcagatgaaggagctcggcatcggcatctcgacctccgtcggcatcggcggcgatccgatcaacggctcctccttcctcgaccacctcgctctgttcgagcaggatcccgagacggaagccgtgctgatgattggcgagatcggcggtccgcaggaggccgaggcctcggcctggatcaaggagaacttttccaagcccgtgatcggcttcgtggcgggcctcaccgcccccaagggccgccgcatggggcatgccggcgcaatcatctcggcgaccggcgacagcgccgcggagaaggccgagatcatgcgctcctatggcctgaccgtggcgcccgatccgggctccttcggcagcaccgtggccgacgtgctcgcccgcgcggcgtga

SEQ ID NO: 59 - protein, 296 aa, Methylobacterium extorquens
MSILIDEKTPILVQGITGDKGTFHAKEMIAYGSNVVGGVTPGKGGKTHCGVPVFNTVKEAVEATGATTSITFVAPPFAADAIMEAADAGLKLVCSITDGIPAQDMMRVKRYLRRYPKEKRTMVVGPNCAGIISPGKSMLGIMPGHIYLPGKVGVISRSGTLGYEAAAQMKELGIGISTSVGIGGDPINGSSFLDHLALFEQDPETEAVLMIGEIGGPQEAEASAWIKENFSKPVIGFVAGLTAPKGRRMGHAGAIISATGDSAAEKAEIMRSYGLTVAPDPGSFGSTVADVLARAA

SEQ ID NO: 60 - DNA, 891 bp, Ruegeria pomeroyi; identical to SEQ ID NO: 29
  CDS (1)..(891)

SEQ ID NO: 61 - protein, 296 aa, Ruegeria pomeroyi; identical to SEQ ID NO: 30

SEQ ID NO: 62 - DNA, 882 bp, Ralstonia eutropha
  CDS (1)..(882)
atgtcgattctgatcaacaaagacaccaaggtcatcacccaggggatcaccggtaaaactggccagttccacacccgcggctgccgcgactacgccaacggcaagaactgctttgttgctggcgtgaacccgaagaaggccggcgaagacttcgaaggcatccccatctacgcaaccgtcaaggacgccaaggcgcaaaccggcgcaagcgtgtcggtcatctacgtgccgcccgcaggcgctgctgacgcgatctgggaagctgtcgaagccgaactggatctggtggtctgcatcaccgaaggcatccccgtgcgcgacatgatgatggtcaaggacaagatgcgtaaggccggcagcaagactctgctgctgggtccgaactgcccgggcctgatcacgccggacgaaatcaagatcggcatcatgccgggtcacatccaccgcaagggccgcatcggcgtggtgtcgcgctcgggcacgctgacgtacgaagccgtgggccagctcaccgcgctgggcctgggccagtcgtcggcagttggtatcggcggcgacccgatcaacggtctgaagcacatcgacgtgatgaagatgttcaacgacgatccggaaacggacgccgtggtcatgatcggtgagatcggcggtccggacgaagccaacgcggcccactggatcaaggacaacatgaagaagccggtggttggcttcatcgctggcgtgaccgcgcctccgggcaagcgcatgggccacgctggcgcgctgatctcgggcggtgccgacacggcgcaagccaagctggacatcatggaagcctgcggcatcaagaccaccaagaacccgtcggaaatggcgcgtctgctgaaggcgatgctgtaa

SEQ ID NO: 63 - protein, 293 aa, Ralstonia eutropha
MSILINKDTKVITQGITGKTGQFHTRGCRDYANGKNCFVAGVNPKKAGEDFEGIPIYATVKDAKAQTGASVSVIYVPPAGAADAIWEAVEAELDLVVCITEGIPVRDMMMVKDKMRKAGSKTLLLGPNCPGLITPDEIKIGIMPGHIHRKGRIGVVSRSGTLTYEAVGQLTALGLGQSSAVGIGGDPINGLKHIDVMKMFNDDPETDAVVMIGEIGGPDEANAAHWIKDNMKKPVVGFIAGVTAPPGKRMGHAGALISGGADTAQAKLDIMEACGIKTTKNPSEMARLLKAML

SEQ ID NO: 64 - DNA, 870 bp, Salmonella enterica
  CDS (1)..(870)
atgtccgttttaatcgataaaaacactaaagttatctgccagggctttaccggtagccaggggacattccactccgagcaggcgattgcgtatggtacgcagatggtgggaggcgtgacgccgggcaaaggcggcactacccatctggggctgccggtattcaacactgtacgtgaagcggtagaggcgacgggcgcgacggcgtcagttatctacgtaccggcgccgttttgcaaagactctattctggaagccatcgacgcgggcatcaaactgattatcaccatcactgaaggtatcccgacgctggacatgctgaccgtgaaagtgaaactggatgaagcgggtgtgcgcatgattggtccgaactgtccaggtgttatcaccccgggtgaatgtaaaatcggcatcatgccgggccatattcacaaaccaggaaaagtgggaattgtctcccgctctggtacgctgacctatgaagcggttaagcagaccaccgattacggtttcggccagtctacctgtgtcggcatcggcggtgaccccatccctggctctaacttcatcgacatcctgaaattgttccaggaagatccgcagaccgaagcgatcgtgatgatcggtgaaatcggcggtagcgcggaagaagaagcggcggcgtatattaaagatcatgtgactaagccggttgtgggttacatcgccggtgtgaccgcgccgaaaggcaagcgtatgggccatgcgggtgccattattgccggtggtaaaggcactgcggatgagaaattcgccgcgctggaagccgcaggcgtcaaaaccgttcgtagcctcgccgatatcggcgaagcgctgaaagcgattataaagtaa

SEQ ID NO: 65 - protein, 289 aa, Salmonella enterica
MSVLIDKNTKVICQGFTGSQGTFHSEQAIAYGTQMVGGVTPGKGGTTHLGLPVFNTVREAVEATGATASVIYVPAPFCKDSILEAIDAGIKLIITITEGIPTLDMLTVKVKLDEAGVRMIGPNCPGVITPGECKIGIMPGHIHKPGKVGIVSRSGTLTYEAVKQTTDYGFGQSTCVGIGGDPIPGSNFIDILKLFQEDPQTEAIVMIGEIGGSAEEEAAAYIKDHVTKPVVGYIAGVTAPKGKRMGHAGAIIAGGKGTADEKFAALEAAGVKTVRSLADIGEALKAIIK

SEQ ID NO: 66 - DNA, 870 bp, Escherichia coli
  CDS (1)..(870)
atgtctattttgattgataagaatactaaggttatttgtcaaggatttactggatctcaaggaacttttcattctgaacaagctattgcttatggaactaagatggttggaggagttactcctggaaagggaggaactactcatttgggattgcctgtttttaatactgttagagaagctgttgctgctactggagctactgcttctgttatttatgttcctgctcctttttgtaaggattctattttggaagctattgatgctggaattaagttgattattactattactgaaggaatacctactttggatatgttgactgttaaggttaagttggatgaagctggagttagaatgattggacctaattgtcctggagttattactcctggagaatgtaagattggaatacaacctggacatattcataagcctggaaaggttggaattgtttctaggtcgggaactttgacttatgaagctgttaagcaaactactgattatggatttggacaatctacttgtgttggaattggaggagatcctattcctggatctaattttattgatattttggaaatgtttgaaaaggatcctcaaactgaagctattgttatgattggagaaattggaggatctgctgaagaagaagctgctgcttatattaaggagcatgttactaagcctgttgttggatatattgctggagttactgctcctaagggaaagagaatgggacatgctggagctattattgctggaggaaagggaactgctgatgaaaagtttgctgctttggaagctgctggagttaagactgttagatcgttggctgatattggagaagctttgaagactgttttgaagtaa

SEQ ID NO: 67 - protein, 289 aa, Escherichia coli
MSILIDKNTKVICQGFTGSQGTFHSEQAIAYGTKMVGGVTPGKGGTTHLGLPVFNTVREAVAATGATASVIYVPAPFCKDSILEAIDAGIKLIITITEGIPTLDMLTVKVKLDEAGVRMIGPNCPGVITPGECKIGIQPGHIHKPGKVGIVSRSGTLTYEAVKQTTDYGFGQSTCVGIGGDPIPGSNFIDILEMFEKDPQTEAIVMIGEIGGSAEEEAAAYIKEHVTKPVVGYIAGVTAPKGKRMGHAGAIIAGGKGTADEKFAALEAAGVKTVRSLADIGEALKTVLK

SEQ ID NO: 68 - DNA, 38 bp, Artificial Sequence
  Forward Primer - Ruegeria pomeroyi mtkAB
ggatccgaattcgatggacattcacgaatatcaggcca

SEQ ID NO: 69 - DNA, 38 bp, Artificial Sequence
  Reverse Primer - Ruegeria pomeroyi mtkAB
tgcggccgcaagcttacgccgcctccctcatgaccctg

SEQ ID NO: 70 - DNA, 38 bp, Artificial Sequence
  Forward Primer - Chloroflexus auriantacus smtAB
ggatccgaattcgatgccccccacaggagaagaaccat

SEQ ID NO: 71 - DNA, 38 bp, Artificial Sequence
  Reverse Primer - Chloroflexus auriantacus smtAB
tgcggccgcaagcttatatcacccgctttgaacgcaga

SEQ ID NO: 72 - DNA, 38 bp, Artificial Sequence
  Forward Primer - Haloarcula marismortui sucCD
ggatccgaattcgatgcgcttgcacgaataccaggcga

SEQ ID NO: 73 - DNA, 37 bp, Artificial Sequence
  Reverse Primer - Haloarcula marismortui sucCD
tgcggccgcaagcttacagtaggtcttcgacgtggtc

SEQ ID NO: 74 - DNA, 38 bp, Artificial Sequence
  Forward Primer - Iodomarina loihiensis sucCD
ggatccgaattcgatgaatttgcatgagtatcagggta

SEQ ID NO: 75 - DNA, 38 bp, Artificial Sequence
  Reverse Primer - Iodomarina loihiensis sucCD
tgcggccgcaagcttaccagccagttttctctgcaaca

SEQ ID NO: 76 - DNA, 38 bp, Artificial Sequence
  Forward Primer - Klebsiella pneumoniae sucCD
ggatccgaattcgatgaacttacatgaatatcaggcaa

SEQ ID NO: 77 - DNA, 38 bp, Artificial Sequence
  Reverse Primer - Klebsiella pneumoniae sucCD
tgcggccgcaagcttacttgatgatagctttcagcgct

SEQ ID NO: 78 - DNA, 38 bp, Artificial Sequence
  Forward Primer - Methylococcus capsulatus sucCD-2
ggatccgaattcgatgaatatccatgagtaccaggcca

SEQ ID NO: 79 - DNA, 38 bp, Artificial Sequence
  Reverse Primer - Methylococcus capsulatus sucCD-2
tgcggccgcaagcttagaatctgattccgtgttcctgc

SEQ ID NO: 80 - DNA, 38 bp, Artificial Sequence
  Forward Primer - Methylobacillus flagellatus sucCD
ggatccgaattcgatgaatttgcatgagtatcaggcca

SEQ ID NO: 81 - DNA, 38 bp, Artificial Sequence
  Reverse Primer - Methylobacillus flagellatus sucCD
tgcggccgcaagcttattgcttggcctggattgcaacc

SEQ ID NO: 82 - DNA, 38 bp, Artificial Sequence
  Forward Primer - Pseumonas syringae sucCD
ggatccgaattcgatgaatcttcacgagtatcagggta

SEQ ID NO: 83 - DNA, 38 bp, Artificial Sequence
  Reverse Primer - Pseumonas syringae sucCD
tgcggccgcaagcttacttggtcggccaaccggtcagc

SEQ ID NO: 84 - DNA, 38 bp, Artificial Sequence
  Forward Primer - Staphylococcus aureus sucCD
ggatccgaattcgatgaatatccacgagtatcaaggta

SEQ ID NO: 85 - DNA, 38 bp, Artificial Sequence
  Reverse Primer - Staphylococcus aureus sucCD
tgcggccgcaagcttatttattaacagttaataatgct

SEQ ID NO: 86 - DNA, 38 bp, Artificial Sequence; identical to SEQ ID NO: 76
  Forward Primer - Salmonella enterica sucCD

SEQ ID NO: 87 - DNA, 38 bp, Artificial Sequence
  Reverse Primer - Salmonella enterica sucCD
tgcggccgcaagcttattttataattgctttcagcgct

SEQ ID NO: 88 - DNA, 38 bp, Artificial Sequence
  Forward Primer - Rhodobacter sphaeroides sucCD
ggatccgaattcgatgaacatccatgaataccaggcga

SEQ ID NO: 89 - DNA, 38 bp, Artificial Sequence
  Reverse Primer - Rhodobacter sphaeroides sucCD
tgcggccgcaagcttatttgccgagcgcctgcatcacg

SEQ ID NO: 90 - DNA, 38 bp, Artificial Sequence
  Forward Primer - Bacillus subtilis sucCD
ggatccgaattcgatgaatatccatgagtaccagggaa

SEQ ID NO: 91 - DNA, 38 bp, Artificial Sequence
  Reverse Primer - Bacillus subtilis sucCD
tgcggccgcaagcttaatgcgttttgcaagtttcgaac

SEQ ID NO: 92 - DNA, 38 bp, Artificial Sequence
  Forward Primer - Pseudoalteromonas atlantica sucCD
ggatccgaattcgatgaatttgcatgaatatcagggta

SEQ ID NO: 93 - DNA, 38 bp, Artificial Sequence
  Reverse Primer - Pseudoalteromonas atlantica sucCD
tgcggccgcaagcttaccaaccggtaatttcgcgaaga

SEQ ID NO: 94 - DNA, 38 bp, Artificial Sequence
  Forward Primer - Colwellia psychrerythraea sucCD
ggatccgaattcgatgaatttgcatgaataccaagcga

SEQ ID NO: 95 - DNA, 38 bp, Artificial Sequence
  Reverse Primer - Colwellia psychrerythraea sucCD
tgcggccgcaagcttaccaacctgttttctctttaagt

SEQ ID NO: 96 - DNA, 38 bp, Artificial Sequence
  Forward Primer - Ralstonia eutropha sucCD
ggatccgaattcgatgaatatccatgagtatcaaggca

SEQ ID NO: 97 - DNA, 38 bp, Artificial Squence
  Reverse Primer - Ralstonia eutropha sucCD
tgcggccgcaagcttacagcattgccttcagcaggcgg

SEQ ID NO: 98 - DNA, 38 bp, Artificial Sequence; identical to SEQ ID NO: 76
  Forward Primer - Escherichia coli sucCD

SEQ ID NO: 99 - DNA, 38 bp, Artificial Sequence
  Reverse Primer - Escherichia coli sucCD
tgcggccgcaagcttatttcagaacagttttcagtgct

SEQ ID NO: 100 - DNA, 30 bp, Artificial Sequence
  Forward Primer - E. coli sucCD mutation x
aacgattgataccggcgaagatgttaacca

SEQ ID NO: 101 - DNA, 30 bp, Artificial Sequence
  Reverse Primer - E. coli sucCD mutation x
cttcgccggtatcaatcgttgcgacctgat

SEQ ID NO: 102 - DNA, 24 bp, Artificial Sequence
  Forward Primer - E. coli sucCD mutation y
aacgcctgcgcagttcgggccgat

SEQ ID NO: 103 - DNA, 24 bp, Artificial Sequence
  Forward Primer - E. coli sucCD mutation y
aacgcctgcgcagttcgggccgat

SEQ ID NO: 104 - DNA, 24 bp, Artificial Sequence
  Reverse Primer - E. coli sucCD mutation y
cgaactgcgcaggcgttatcactc

SEQ ID NO: 105 - DNA, 24 bp, Artificial Sequence
  Forward Primer - E. coli sucCD mutation z
ttcatagcccagtgtaccggaacg

SEQ ID NO: 106 - DNA, 24 bp, Artificial Sequence
  Reverse Primer - E. coli sucCD mutation z
tacactgggctatgaagcggttaa